(19) United States
(12) Patent Application Publication
(10) Pub. No.: US 2007/0037202 A1
(43) Pub. Date: Feb. 15, 2007

(54) Potentiation of cancer therapies by ZNF217 inhibition

(75) Inventors: Colin Collins, San Rafael, CA (US); Guiqing Huang, San Bruno, CA (US); Joe W. Gray, San Francisco, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Regents of the University of California, Oakland, CA (US)

(21) Appl. No.: 11/583,305

(22) Filed: Oct. 18, 2006

Related U.S. Application Data
(62) Division of application No. 10/349,627, filed on Jan. 22, 2003.
(60) Provisional application No. 60/351,530, filed on Jan. 22, 2002.

(51) Int. Cl. C12Q 1/68; A61K 31/704

(52) U.S. Cl. 435/6; 514/34

(57) ABSTRACT

This invention provides methods, reagents and kits for treating cancer in a patient or subject, e.g., a human. Accordingly, the present methods can be used to monitor the efficacy of a cancer treatment and to treat cancer, e.g., by inhibiting the expression and/or activity of ZNF217 in a neoplastic cell.

### CROSS-REFERENCES TO RELATED APPLICATIONS

The present application is a divisional of U.S. patent application Ser. No. 10/349,627, filed Jan. 22, 2003, which claims benefit of priority to U.S. Provisional Patent Application No. 60/351,530, filed on Jan. 22, 2002, which is incorporated in its entirety for all purposes.

### TECHNICAL FIELD

This invention relates generally to cancer therapy, such as breast cancer. In particular, the present invention provides reagents and methods for treating cancers that are associated with amplification or overexpression of ZNF217 nucleic acids and polypeptides.

### BACKGROUND

Chromosome abnormalities are often associated with genetic disorders, degenerative diseases, and cancer. The deletion or multiplication of copies of whole chromosomes and the deletion or amplifications of chromosomal segments or specific regions are common occurrences in cancer (Smith, H. S. et al., 1991, Breast Cancer Res. Treat. 18:Suppl. 1:51-54; van de Vijer, M. J. et al., 1991, Biochim. Biophys. Acta. 1072:33-50).

One of the amplified regions found in studies of breast cancer cells is on chromosome 20, specifically, 20q13.2 (see, e.g., Gray, J. et al., WO98/02539). Amplification of 20q13.2 was subsequently found to occur in a variety of tumor types and to be associated with aggressive tumor behavior. Increased 20q13.2 copy number has been found in 40% of breast cancer cell lines and 18% of primary breast tumors (Kalliioniemi, A. et al., 1994, Proc. Natl. Acad. Sci. U.S.A. 91:2156-2160). Copy number gains at 20 q13.2 have also been reported in greater than 25% of cancers of the ovary (Iwabuchi, H. et al., 1995, Cancer Res. 55:6172-6180), colon (Schlegel, J. et al., 1995, Cancer Res. 55:6002-6005), head-and-neck (Bockmuhl, U. et al., 1996, Laryngor. 75:408-414), brain (Mohapatra, G. et al., 1995, Genes Chromosomes Cancer 13:86-93), and pancreas (Solinas-Toldo, 1996, Genes Chromosomes Cancer 20:399-407).

The ZNF217 gene locus at 20q13.2 is amplified in approximately 20% to 30% of early stage breast tumors (Waldman, F. M. et al., 2000, Journal of the National Cancer Institute 92:313-320) and high level amplification is associated with a 50% decrease in disease free survival (Courjal, F. et al., 1996, Br J Cancer 74:1984-9; Isola et al., 1995, Am J Pathol. 147:905-11; Tanner, M. M. et al., 1995, Clin Cancer Res 1: 1455-61). Increased 20q13.2 copy number is observed upon immortalization of uroepithelial cells (Cuthill, S. et al., 1999, Genes Chromosomes Cancer 26:304-11; Savelieva, E. et al., 1997, Oncogene 14:551-60), and kerotinocytes (Solinas-Toldo, S. et al., 1997, Proc Natl Acad Sci U.S.A. 94:3854-9) and ectopic expression of ZNF217 results HMEC immortalization without an increase in 20q13.2 copy number (Nonet, G. H. et al., 2001, Cancer Res 61:1250-4). The ZNF217 gene product resembles a kruppel-like transcription factor (Collins, C. et al., 1998, Proc Natl Acad Sci U.S.A. 95:8703-8) and localizes predominantly to the nucleus (Collins, C. et al., 2001, Genome Res 11:1034-42) and coimmunoprecipitates with histone deacetylase 1 (HDAC1)(You, A., et al., 2001, Proc Natl Acad Sci U.S.A. 98:1454-8) suggesting it can function as a transcriptional repressor.

Definitive functional characterization of ZFN217 would be an important step in the diagnosis and prognosis of cancer. As described herein, this discovery has provided novel and badly needed therapeutic tools for many types of cancers including breast cancer.

### SUMMARY

This invention provides methods, reagents and kits for treating cancer in a patient or subject, e.g., a human. Accordingly, the present methods can be used to monitor the efficacy of a cancer treatment and to treat cancer, e.g., by inhibiting the expression and/or activity of ZNF217 in a neoplastic cell.

In one aspect, the present invention provides a method of inhibiting the growth of a neoplastic cell, the method comprising contacting the cell with a chemotherapeutic drug and a ZNF217 inhibitor, thereby inhibiting the growth of the neoplastic cell. In some such methods, the ZNF217 inhibitor comprises an antisense nucleic acid. In some embodiments, the ZNF217 inhibitor comprises an siRNA nucleic acid. In some such methods, the chemotherapeutic drug is a topoisomerase inhibitor. In some such methods, the chemotherapeutic drug is doxorubicin. In some such methods, the chemotherapeutic drug activates apoptotic pathways.

In another aspect, the present invention provides a method of potentiating antineoplastic therapy in a patient, the method comprising the steps of:(a) administering a ZNF217 inhibitor to the patient; and (b) administering an antineoplastic therapy to the patient, thereby potentiating antineoplastic therapy in the patient. In some such methods, the ZNF217 inhibitor comprises an antisense nucleic acid. In some such methods, the ZNF217 inhibitor comprises an siRNA nucleic acid. In some such methods, the antineoplastic therapy comprises administering radiation to the patient. In some such methods, the antineoplastic therapy comprises administering a chemotherapeutic drug to the patient.

In another aspect, the present invention provides a method of identifying agents that promote cell death in a mammalian cell, the method comprising the steps of:(a) providing a mammalian cell engineered to overexpress ZNF217; (b)contacting the cell with a test agent; and (c) assaying for the effect of the test agent on death of the cell, thereby identifying agents that promote death in a mammalian cell. In some such methods, the method further comprises the step of contacting the cell with a chemotherapeutic drug. In some such methods, the chemotherapeutic drug is a drug that promotes apoptosis. In some such methods, the chemotherapeutic drug is a topoisomerase inhibitor. In some such methods, the effect of the test agent on death of the cell is assayed by measuring the incidence of apoptosis. In some such methods, the effect of the test agent on death of the cell is assayed by measuring the activity of p53. In some such methods, the cell is transfected with a nucleic acid construct encoding ZNF217. In some such methods, the cell is in culture. In some such methods, the cell is in a mouse.

In another aspect, the present invention provides a method of identifying a compound that modulates activity of a ZNF217 polypeptide, the method comprising the steps of:(a) contacting the ZNF217 polypeptide with the compound, wherein the ZNF217 polypeptide comprises at least 85% amino acid sequence identity to the amino acid sequence of ZNF217 (GenBank Accession No. AAC39895; RefSeq Accession ID No. NP_{—}006517); and (b)determining the functional effect of the compound on the ZNF217 polypeptide thereby identifying a compound that modulates activity of ZNF217 polypeptide. In some such methods, the polypeptide is linked to a solid phase. In some such methods, the polypeptide is covalently linked to a solid phase.

In some such methods, the polypeptide is expressed in a cell. In some such methods, the polypeptide is amplified in the cell compared to normal. In some such methods, the polypeptide has an amino acid sequence of ZNF217 (GenBank Accession No. AAC39895; RefSeq Accession ID No. NP_{—}006517).

In another aspect, the present invention provides a method of identifying agents that modulate the activity of a chemotherapeutic drug, the method comprising the steps of:(a) providing a mammalian cell engineered to overexpress ZNF217; (b) contacting the cell with a test agent and a chemotherapeutic drug; and (c) assaying for the effect of the test agent on the activity of the chemotherapeutic drug, thereby identifying agents that modulate the activity of the chemotherapeutic drug. In some such methods, the chemotherapeutic drug is a topoisomerase inhibitor. In some such methods, the chemotherapeutic drug is doxorubicin.

In another aspect, the present invention provides a method of monitoring the efficacy of a cancer treatment, the method comprising detecting the level of a ZNF217 polypeptide or polynucleotide in a biological sample from a patent undergoing treatment for cancer, wherein a reduced level of the ZNF217 polypeptide or polynucleotide in the biological sample compared to the level in a biological sample from the patient prior to, or earlier in, the treatment is indicative of efficacious treatment. In some such methods, the cancer is breast cancer. In some such methods, the patient is a human.

### BRIEF DESCRIPTION OF THE DRAWINGS

FIG. 1. 1A. ZNF217 is expressed at high level in transfected HeLa cells compared to non transfected HeLa cells. Transcripts from the endogenous and transfected fusion cDNA are evident. The level of ZNF217-EGFP message is similar to that observed in breast tumors with ZNF217 gene amplification (Collins et al., 1998). 1B. ZNF217-EGFP protein is made in the transfected HeLa cells and localizes predominantly to the nucleus (green). The cytoplasm is marked in one cell by co transfection with prefoldin 4-red fluorescent protein fusion. 1C stable transfection of HeLa cells with a plasmid encoding a ZNF217-GFP fusion results in ˜40% more transfected HeLa cells after 72 hours (crimson bar) than in a parallel culture of control non transfected (white bar) or EGFP vector transfected HeLa cells (blue bar).

FIG. 2. Significantly less (˜40%) spontaneous cell death occurs in HeLa cells transfected with the ZNF217-EGFP cDNA (crimson bar) than occurs in parallel cultures of non transfected control HeLa cells (blue bars) or EGFP vector transfected HeLa cells (white bar).

FIG. 3. The ZNF217-GFP construct protects HeLa cells against doxorubicin induced cell death. Control non transfected HeLa cells (blue bar), ZNF217-EGFP transfected (crimson bar) and EGFP vector transfected HeLa cells (white bar) were exposed to varying amounts of doxorubicin for 72 hours and cell death measured by propidium iodide nuclear staining using FACS analysis. Transfection of the ZNF217-EGFP fusion confers significant resistant to doxorubicin on HeLa cells.

FIG. 4. Human breast cancer cell lines with and without amplification and/or overexpression of ZNF217 were analyzed for sensitivity to doxorubicin. Two cells lines with overexpression of ZNF217 MCF7 (red bar) and 600MPE (white bar) and one with low-level ZNF217 expression HBL100 (blue bar) are shown all three cell lines are wild type for TP53. Breast cancer cell lines with high relative expression of ZNF217 are three to five-fold more resistant to doxorubicin than HBL100 that expresses little ZNF217.

FIG. 5. Transfection of HBL100 with ZNF217-EGFP confers a level of doxorubicin resistance on HBL100 similar to that observed in 600MPE and MCF7 cell lines that express high level of endogenous ZNF217. ZNF217-EGFP transfected HBL100 cells indicated by the crimson bar and non transfected HBL 100 controls are indicated by a blue bar.

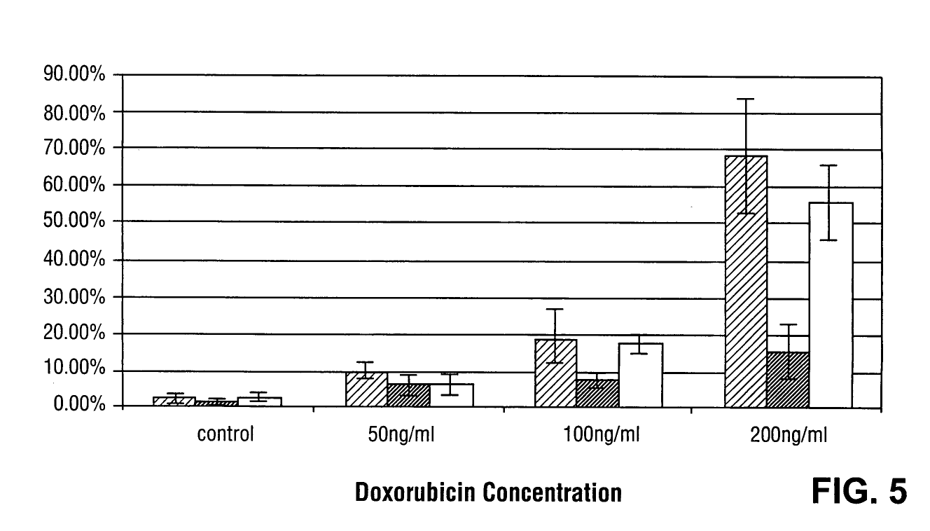

FIG. 6. HMEC cells immortalized by retroviral transduction of ZNF217 are more resistant to doxorubicin than the control non transduced parental HMECs. Apoptotic response to doxorubicin exposure in parental non transduced HMEC (blue bar) and ZNF217 transduced and immortalized cells (crimson bar) (Nonet et al., 2001, supra). The difference in cell killing is significant for 200 ng/ml p=0.048.

FIG. 7. A. ZNF217 transfected cells protects against apoptosis induced by doxorubicin compared to non-transfected cells. B. Apoptotic HeLa cells showed bright red annexin V staining on the cellular membrane but the ZNF217-GFP transfected HeLa cells (green) do not stain with annexin V. C. Apoptotic HBL 100 show annexin V staining on the cellular membrane but ZNF217-GFP transfected HBL 100 cells (green) do not.

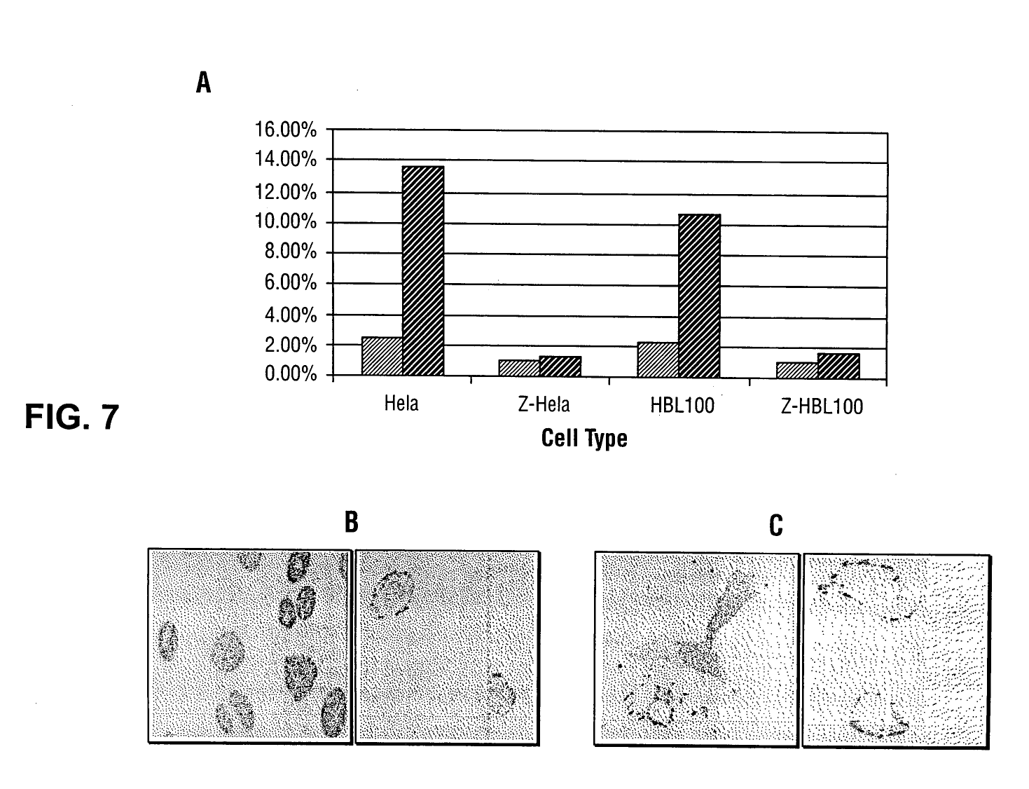

FIG. 8. ZNF217 protects HeLa and HBL100 cells from apoptosis induced by functional inactivation of the telomere binding protein TRF2. HeLa and.HBL100 cells transfected with a plasmid encoding a TRF2 dominant negative telomere binding protein induces rapid apoptosis (Karlseder, J. et al., 1999, Science 283:1321-5) and blue and orange bars. The same cells co transfected with TRF2 and ZNF217-EGFP are resistant to apoptosis triggered by functional inactivation of TRF2 (HeLa/crimson and HBL100/blue). Consistent with these results breast cancer cell lines (MCF7/green and 600MPE/purple) with high endogenous ZNF217 expression are more resistant to functional inactivation of TRF2 than HBL100 which expressed low levels of ZNF217. ZNF217 protects HeLa and HBL100 cells from apoptosis induced by functional inactivation of the telomere binding protein TRF1.

FIG. 9. HBL100 cells transfected with ZNF217-EGFP exposed to doxorubicin for 16 hours are more resistant to doxorubicin than non transfected controls. After exposure the cells were washed and allowed to grow for 11 days. At the end of 11 days there were 3.35-fold more ZNF217-GFP transfected cells than non transfected cells.

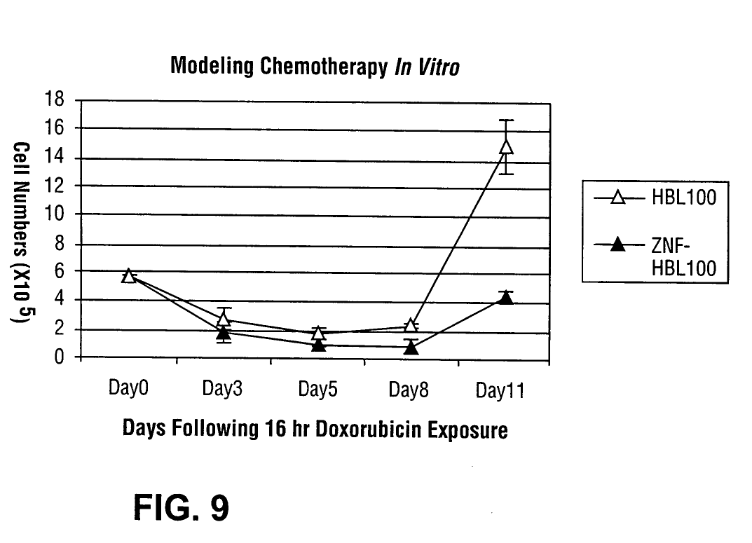

FIG. 10. A higher percentage of HBL100 cells transfected with ZNF217-EGFP and a ZNF217 siRNA and then exposed to doxorubicin went through apoptosis than control cells. After exposure the cells were washed and allowed to grow for 11 days. About 80% of control HBL100 cells stained with annexin following Doxorubin treatment. Less than 40% of cells transfected with NF217 and treated with Doxorubin stained. Of ZNF217-transfected cells also transfected with ZNF217 siRNA and treated with doxorubin, nearly 100% were stained with annexin.

FIG. 11. HBL100 cells were exposed to doxorubicin and triciribene phosphate. Increasing concentrations of triciribene phosphate, in combination with doxorubicin resulted in increased annexin staining. Those cells not also treated with doxorubicin did not display significant staining.

FIG. 12. HBL100 cells transfected with ZNF217-EGFP were exposed to doxorubicin and triciribene phosphate. Increasing concentrations of triciribene phosphate, in combination with doxorubicin resulted in increased annexin staining. Those cells not also treated with doxorubicin did not display significant staining.

### DETAILED DESCRIPTION

I. Introduction

The present invention provides methods of production of ZNF217. The sequences can be used for the identification of molecules that associate with and/or modulate the activity of ZNF217, or for the diagnosis of cancer or other diseases or conditions associated with ZNF217 amplification or ZNF217 activity or expression. In one aspect, the invention is based upon the discovery that the ZNF217 gene is overexpressed and/or amplified in cancer cells, particularly breast cancer cells. Accordingly, the present methods can be used to monitor the efficacy of a cancer treatment, and to treat cancer, e.g., by inhibiting the expression and/or activity of ZNF217 in a cancer cell.

The invention also provides methods of screening for modulators, e.g., activators, inhibitors, stimulators, enhancers, and the like, of ZNF217 nucleic acids and proteins. Such modulators can affect ZNF217 activity, e.g., by modulating ZNF217 transcription, translation, mRNA or protein stability; by altering the interaction of ZNF217 with other molecules (e.g., ZNF217 regulated genes); or by affecting ZNF217 protein activity. In one embodiment, compounds are screened, e.g., using high throughput screening (HTS), to identify those compounds that can bind to and/or modulate the activity of an isolated ZNF217 polypeptide or fragment thereof. In another embodiment, ZNF217 proteins are recombinantly expressed in cells, and the modulation of ZNF217 is assayed by using any measure of ZNF217 function.

In numerous embodiments, a ZNF217 polynucleotide or polypeptide is introduced into a cell, in vivo or ex vivo, and the ZNF217 activity in the cell is thereby modulated. For example, a polynucleotide encoding a full length ZNF217 polypeptide can be introduced into a population of cells.

In certain embodiments, monoclonal or polyclonal antibodies directed to ZNF217, or subfragment or domain of ZNF217, will be administered to a patient to inhibit the activity of ZNF217 in cells. Such embodiments are useful, e.g., in the treatment of a disease or disorder associated with ZNF217 activity.

The present invention also provides methods for detecting ZNF217 nucleic acid and protein expression. ZNF217 polypeptides can also be used to generate monoclonal and polyclonal antibodies useful for the detection of ZNF217-expressing cells or for the amelioration of ZNF217 activity. Cells that express ZNF217 can also be identified using techniques such as reverse transcription and amplification of mRNA, isolation of total RNA or poly A+RNA, northern blotting, dot blotting, in situ hybridization, RNase protection, S1 digestion, probing DNA microchip arrays, western blots, and the like.

Nucleotide and amino acid sequence information for ZNF217 are also used to construct models of ZNF217 proteins. These models are subsequently used to identify compounds that can activate or inhibit ZNF217 proteins. Such compounds that modulate the activity of ZNF217 genes or proteins can be used to investigate the physiological role of ZNF217 genes.

The present invention also provides assays, preferably high throughput screening (HTS) assays, to identify compounds or other molecules that interact with and/or modulate ZNF217. In certain assays, a particular domain of ZNF217 is used, e.g., a conserved domain.

The present invention also provides methods to treat diseases or conditions associated with ZNF217 activity. For example, ZNF217 activity and/or expression can be altered in cells of a patient with a ZNF217-associated disease. In particular, the invention provides for methods of treating cancer.

Methods of the invention directed to treating cancer typically involve detecting the presence of ZNF217 in a biological sample taken from a patient. In certain embodiments, a level of ZNF217 in a biological sample will be compared with a control sample taken from a cancer-free patient or, preferably, with a value expected for a sample taken from a cancer-free patient. A control sample can also be obtained from normal tissue from the same patient that is suspected of having cancer.

The ability to detect cancer cells by virtue of an increased level of ZNF217 is useful for any of a large number of applications. For example, an increased level of ZNF217 in cells of a patient can be used, alone or in combination with other diagnostic methods, to diagnose cancer in the patient or to determine the propensity of a patient to develop cancer over time. The detection of ZNF217 can also be used to monitor the efficacy of a cancer treatment. For example, a level of a ZNF217 polypeptide or polynucleotide after an anti-cancer treatment is compared to the level in the patient before the treatment. A decrease in the level of the ZNF217 polypeptide or polynucleotide after the treatment indicates efficacious treatment.

An increased level or diagnostic presence of ZNF217 can also be used to influence the choice of anti-cancer treatment in a patient, where, for example, the level of ZNF217 increase directly correlates with the aggressiveness of the anti-cancer therapy. For example, an increased level of ZNF217 in tumor cells can indicate that the use of an agent that decreases proliferation would be effective in treating the tumor.

In addition, the ability to detect cancer cells can be used to monitor the number or location of cancer cells in a patient, in vitro or in vivo, for example, to monitor the progression of the cancer over time. In addition, the level or presence or absence of ZNF217 can be statistically correlated with the efficacy of particular anti-cancer therapies or with observed prognostic outcomes, thereby allowing for the development of databases based on a statistically-based prognosis, or a selection of the most efficacious treatment, can be made in view of a particular level or diagnostic presence of ZNF217.

The present invention also provides methods for treating cancer. In certain embodiments, the proliferation of a cell with an elevated level of ZNF217 polynucleotides, polypeptides, or polypeptide activity is inhibited. In other embodiments, ZNF217 expression is not elevated compared to normal, but ZNF217 activity, for example, functions at the cell surface membrane, can be blocked or inhibited to prevent tumor cell growth, migration, or metastasis. Proliferation and/or migration is decreased by, for example, contacting the cell with an inhibitor of ZNF217 transcription or translation, or an inhibitor of the activity of a ZNF217 polypeptide. Such inhibitors include, but are not limited to, siRNA, polynucleotides, ribozymes, antibodies, dominant negative ZNF217 polypeptides, and small molecule inhibitors of ZNF217 activity.

The present methods can be used to diagnose, determine the prognosis for, or treat, any of a number of types of cancers. In preferred embodiments, the cancer is an epithelial cancer, e.g., prostate, lung, breast, colon, kidney, stomach, bladder, or ovarian cancer, or any cancer of the gastrointestinal tract. In a presently preferred embodiment, the cancer is prostate cancer.

The methods of this invention can be used in animals including, for example, primates, canines, felines, murines, bovines, equines, ovines, porcines, lagomorphs, etc, as well as in humans. In a preferred embodiment, the mammal is a human.

Kits are also provided for carrying out the herein-disclosed therapeutic methods.

II. Definitions

As used herein, the following terms have the meanings ascribed to them unless specified otherwise.

The terms “patient” or “subject” are used interchangeably and refer to mammals such as human patients and non-human primates, as well as experimental animals such as rabbits, rats, and mice, and other animals.

The term “biological sample” as used herein is a sample of biological tissue, fluid, or cells that contains ZNF217 or nucleic acid encoding ZNF217 protein. Such samples include, but are not limited to, tissue isolated from humans. Biological samples can also include sections of tissues such as frozen sections taken for histologic purposes. A biological sample is typically obtained from a eukaryotic organism, preferably eukaryotes such as fungi, plants, insects, protozoa, birds, fish, reptiles, and preferably a mammal such as rat, mice, cow, dog, guinea pig, or rabbit, and most preferably a primate such as chimpanzees or humans.

The term “treating” includes the administration of the compounds or agents of the present invention to prevent or delay the onset of the symptoms, complications, or biochemical indicia of a disease, alleviating the symptoms or arresting or inhibiting further development of the disease, condition, or disorder (e.g., breast cancer). Treatment can be prophylactic (to prevent or delay the onset of the disease, or to prevent the manifestation of clinical or subclinical symptoms thereof) or therapeutic suppression or alleviation of symptoms after the manifestation of the disease.

“Cancer” or “malignancy” are used as synonymous terms and refer to any of a number of diseases that are characterized by uncontrolled, abnormal proliferation of cells, the ability of affected cells to spread locally or through the bloodstream and lymphatic system to other parts of the body (i.e., metastasize) as well as any of a number of characteristic structural and/or molecular features. A “cancerous” or “malignant cell” is understood as a cell having specific structural properties, lacking differentiation and being capable of invasion and metastasis. Examples of cancers are kidney, colon, breast, prostate and liver cancer. (see DeVita, V. et al. (eds.), 2001, CANCER PRINCIPLES AND PRACTICE OF ONCOLOGY, 6^{th} Ed., Lippincott Williams & Wilkins, Philadelphia, Pa.; this reference is herein incorporated by reference in its entirety for all purposes).

“Cancer-associated” refers to the relationship of a nucleic acids and its expression, or lack thereof, or a protein and its level or activity, or lack thereof, to the onset of malignancy in a subject cell. For example, cancer can be associated with expression of a particular gene that is not expressed, or is expressed at a lower level, in a normal healthy cell. Conversely, a cancer-associated gene can be one that is not expressed in a malignant cell (or in a cell undergoing transformation), or is expressed at a lower level in the malignant cell than it is expressed in a normal healthy cell.

As used herein, “neoplastic cells” and “neoplasia” refer to cells which exhibit relatively autonomous growth, so that they exhibit an aberrant growth phenotype characterized by a significant loss of control of cell proliferation. Neoplastic cells comprise cells which can be actively replicating or in a temporary non-replicative resting state (G1 or G0 ); similarly, neoplastic cells can comprise cells which have a well-differentiated phenotype, a poorly-differentiated phenotype, or a mixture of both type of cells. Thus, not all neoplastic cells are necessarily replicating cells at a given timepoint. The set defined as neoplastic cells consists of cells in benign neoplasms and cells in malignant (or frank) neoplasms. Frankly neoplastic cells are frequently referred to as cancer (discussed supra), typically termed carcinoma if originating from cells of endodermal or ectodermal histological origin, or sarcoma if originating from cell types derived from mesoderm.

In the context of the invention, the term “transformation” refers to the change that a normal cell undergoes as it becomes malignant. In eukaryotes, the term “transformation” can be used to describe the conversion of normal cells to malignant cells in cell culture.

“Proliferating cells” are those which are actively undergoing cell division and growing exponentially. “Loss of cell proliferation control” refers to the property of cells that have lost the cell cycle controls that normally ensure appropriate restriction of cell division. Cells that have lost such controls proliferate at a faster than normal rate, without stimulatory signals, and do not respond to inhibitory signals.

The term “apoptosis” and “programmed cell death” (PCD) are used as synonymous terms and describe the molecular and morphological processes leading to controlled cellular self-destruction (see, e.g., Kerr J. F. R. et al., 1972, Br J Cancer. 26:239-257). Apoptotic cell death can be induced by a variety of stimuli, such as ligation of cell surface receptors, starvation, growth factor/survival factor deprivation, heat shock, hypoxia, DNA damage, viral infection, and cytotoxic/chemotherapeutical agents. The apoptotic process is involved in embryogenesis, differentiation, proliferation/homoeostasis, removal of defect and therefore harmful cells, and especially in the regulation and function of the immune system. Thus, dysfunction or disregulation of the apoptotic program is implicated in a variety of pathological conditions, such as immunodeficiency, autoimmune diseases, neurodegenerative diseases, and cancer. Apoptotic cells can be recognized by stereotypical morphological changes: the cell shrinks, shows deformation and looses contact to its neighboring cells. Its chromatin condenses, and finally the cell is fragmented into compact membrane-enclosed structures, called “apoptotic bodies” which contain cytosol, the condensed chromatin, and organelles. The apoptotic bodies are engulfed by macrophages and thus are removed from the tissue without causing an inflammatory response. This is in contrast to the necrotic mode of cell death in which case the cells suffer a major insult, resulting in loss of membrane integrity, swelling and disrupture of the cells. During necrosis, the cell contents are released uncontrolled into the cell's environment what results in damage of surrounding cells and a strong inflammatory response in the corresponding tissue. See, e.g., Tomei L. D. and Cope F. O., eds., 1991, APOPTOSIS: THE MOLECULAR BASIS OF CELL DEATH, Plainville, N.Y.: Cold Spring Harbor Laboratory Press; Isaacs J. T., 1993, Environ Health Perspect. 101(suppl 5):27-33; each of which is herein incorporated by reference in its entirety for all purposes. A variety of apoptosis assays are well known to one of skill in the art (e.g., DNA fragmentation assays, radioactive proliferation assays, DNA laddering assays for treated cells, Fluorescence microscopy of 4′-6-Diamidino-2-phenylindole (DAPI) stained cells assays, and the like).

The term “p53” refers to the p53 gene and its protein product. The p53 protein is a tumor suppressor protein and critical transcriptional activator that causes both G1 and G2 cell cycle arrest when cells are exposed to DNA-damaging agents. The p53 protein is encoded by a gene found on chromosome 17. Mutations in the p53 gene are among the most common genetic alterations observed in human tumor samples and have been estimated to occur in at least 50% of all human tumors (see, e.g., Hollstein, M. et al., 1991, Science 253:49). The p53 protein contains DNA-binding, oligomerization and transcription activation domains. p53 mutants that frequently occur in a number of different human cancers fail to bind the consensus DNA binding site, therefore causing the loss of p53 tumor suppressor activity.

The term “ZNF217” refers to ZNF217 nucleic acid and polypeptide polymorphic variants, alleles, mutants, and interspecies homologs that: (1) have an amino acid sequence that has greater than about 60% amino acid sequence identity, preferably 65%, 70%, 75%, 80%, 85%, 90%, preferably 86%, 87%, 88%, 89%, 90%, 91%, 92%, 93%, 94%, 95%, 96%, 97%, 98% or 99% or greater amino acid sequence identity, preferably over a region of over a region of at least about 50, 100, 200, 500, 1000, or more amino acids, corresponding to the sequence of the naturally occurring ZNF217 gene as, e.g., provided in Collins, C. et al., 1998, Proc Natl Acad Sci U.S.A. 95:8703-8; Gray, J. et al., U.S. Pat. No. 5,801,021; Gray, J. et al., U.S. Pat. No. 5,892,010; Gray, J. et al., U.S. Pat. No. 6,268,184; Gray, J. et al., WO98/02539; and, e.g., in GenBank Accession No.: AF041259, RefSeq Accession ID No. NM_{—}006526; (2) bind to antibodies, e.g., polyclonal antibodies, raised against an immunogen comprising an amino acid sequence corresponding to the sequence of the naturally occurring ZFN217 gene, and conservatively modified variants thereof as, as, e.g., provided in Collins, C. et al., 1998, Proc Natl Acad Sci U.S.A. 95:8703-8; Gray, J. et al., U.S. Pat. No. 5,801,021; Gray, J. et al., U.S. Pat. No. 5,892,010; Gray, J. et al., U.S. Pat. No. 6,268,184; Gray, J. et al., WO98/02539; and, e.g., in GenBank Accession No. AAC39895, RefSeq Accession ID No. NP_{—}006517; (3) specifically hybridize under stringent hybridization conditions to the sequence of the naturally occurring ZFN217 gene and conservatively modified variants thereof; (4) have a nucleic acid sequence that has greater than about 80%, preferably about 85% or 90%, preferably greater than about 96%, 97%, 98%, 99%, or higher nucleotide sequence identity, preferably over a region of over a region of at least about 50, 100, 200, 500, 1000, or more nucleotides, corresponding to the sequence of the naturally occurring ZFN217 gene as, e.g., provided in Collins, C. et al., 1998, Proc Natl Acad Sci U.S.A. 95:8703-8; Gray, J. et al., U.S. Pat. No. 5,801,021; Gray, J. et al., U.S. Pat. No. 5,892,010; Gray, J. et al., U.S. Pat. No. 6,268,184; Gray, J. et al., WO98/02539; and, e.g., in GenBank Accession No. AF041259, RefSeq Accession ID No. NM_{—}006526. A ZNF217 polynucleotide or polypeptide sequence is typically from a mammal including, but not limited to, human, rat, mouse, hamster, cow, pig, horse, sheep, or any mammal. A “ZNF217 polynucleotide” and a “ZNF217 polypeptide” are both either naturally occurring or recombinant. A “ZNF217 protein” or “polypeptide” can comprise naturally occurring or synthetic amino acids, e.g., labeled or otherwise modified amino acids or amino acid analogs. A “ZNF217 protein” will typically contain one or more characteristic protein motifs, any of which can be used independently of other elements normally present in a full-length ZNF217 protein, and will have one or more characteristic activities or properties, e.g.,. A “ZNF217 protein” can refer to any naturally occurring or synthetic ZNF217 polypeptide as described above. The naturally occurring human ZNF217 gene is located at chromosome 20q13.2 based on the Human Genome Project draft sequence data, listed at National Center for Biotechnology Information (NCBI) in LOCUSLINK at LOCUSID7764. A cluster of expressed sequence tags (ESTs) for ZNF217 is found at NCBI in UniGene at UniGene ID number Hs.155040. The ZFN217 gene is annotated to genomic clone rp4-724E16 (GenBank Locus ID No. AL157838).

An “siRNA” or “RNAi” refers to a nucleic acid that forms a double stranded RNA, which double stranded RNA has the ability to reduce or inhibit expression of a gene or target gene when the siRNA expressed in the same cell as the gene or target gene. “siRNA” thus encompasses the double stranded RNA formed by the complementary strands. The complementary portions of the siRNA that hybridize to form the double stranded molecule typically have substantial or complete identity. In one embodiment, an siRNA refers to a nucleic acid that has substantial or complete identity to a target gene and forms a double stranded siRNA. The sequence of the siRNA can correspond to the full length target gene, or a subsequence thereof. Typically, the siRNA is at least about 15-50 nucleotides in length (e.g., each complementary sequence of the double stranded siRNA is 15-50 nucleotides in length, and the double stranded siRNA is about 15-50 base pairs in length, preferable about preferably about 20-30 base nucleotides, preferably about 20-25 nucleotides in length, e.g., 20, 21, 22, 23, 24, 25, 26, 27, 28, 29, or 30 nucleotides in length. siRNAs can be introduced into animals according to any methods, including those of, e.g., U.S. Application 2002/0132788.

A “full length” ZNF217 protein or nucleic acid refers to a ZNF217 polypeptide or polynucleotide sequence, or a variant thereof, that contains all of the elements normally contained in one or more naturally occurring, wild type ZNF217 polynucleotide or polypeptide sequences.

The term “nucleic acid” refers to deoxyribonucleotides or ribonucleotides and polymers thereof in either single- or double-stranded form. Unless specifically limited, the term encompasses nucleic acids containing known analogues of natural nucleotides which have similar binding properties as the reference nucleic acid (e.g., ZNF217) and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences and as well as the sequence explicitly indicated. Specifically, degenerate codon substitutions can be achieved by generating sequences in which the third position of one or more selected (or all) codons is substituted with mixed-base and/or deoxyinosine residues (Batzer et al., 1991, Nucleic Acid Res. 19:5081; Ohtsuka et al., 1985, J. Biol. Chem. 260:2605-2608; and Cassol et al., 1992; Rossolini et al., 1994, Mol. Cell. Probes 8:91-98). The term nucleic acid is used interchangeably with gene, cDNA, and mRNA encoded by a gene.

As used herein a “nucleic acid probe” is defined as a nucleic acid capable of binding to a target nucleic acid (e.g., a nucleic acid associated with cancer) of complementary sequence through one or more types of chemical bonds, usually through complementary base pairing, usually through hydrogen bond formation. As used herein, a probe can include natural (i.e., A, G, C, or T) or modified bases (7-deazaguanosine, inosine, and the like). In addition, the bases in a probe can be joined by a linkage other than a phosphodiester bond, so long as it does not interfere with hybridization. Thus, for example, probes can be peptide nucleic acids in which the constituent bases are joined by peptide bonds rather than phosphodiester linkages. It will be understood by one of skill in the art that probes can bind target sequences lacking complete complementarity with the probe sequence depending upon the stringency of the hybridization conditions.

Nucleic acid probes can be DNA or RNA fragments. DNA fragments can be prepared, for example, by digesting plasmid DNA, or by use of PCR, or synthesized by either the phosphoramidite method described by Beaucage and Carruthers, 1981, Tetrahedron Lett. 22:1859-1862, or by the triester method according to Matteucci, et al., 1981, J. Am. Chem. Soc., 103:3185, both incorporated herein by reference. A double stranded fragment can then be obtained, if desired, by annealing the chemically synthesized single strands together under appropriate conditions, or by synthesizing the complementary strand using DNA polymerase with an appropriate primer sequence. Where a specific sequence for a nucleic acid probe is given, it is understood that the complementary strand is also identified and included. The complementary strand will work equally well in situations where the target is a double-stranded nucleic acid.

A “labeled nucleic acid probe” is a nucleic acid probe that is bound, either covalently, through a linker, or through ionic, van der Waals or hydrogen bonds to a label such that the presence of the probe can be detected by detecting the presence of the label bound to the probe.

The phrase “a nucleic acid sequence encoding” refers to a nucleic acid which contains sequence information for a structural RNA such as rRNA, a tRNA, or the primary amino acid sequence of a specific protein or peptide, or a binding site for a trans-acting regulatory agent. This phrase specifically encompasses degenerate codons (i.e., different codons which encode a single amino acid) of the native sequence or sequences which can be introduced to conform with codon preference in a specific host cell.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or, in the case of cells, to progeny of a cell so modified. Thus, for example, recombinant cells express genes that are not found within the native (non-recombinant) form of the cell or express native genes that are otherwise abnormally expressed, under expressed or not expressed at all.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given polypeptide. For instance, the codons CGU, CGC, CGA, CGG, AGA, and AGG all encode the amino acid arginine. Thus, at every position where an arginine is specified by a codon, the codon can be altered to any of the corresponding codons described without altering the encoded polypeptide. Such nucleic acid variations are “silent substitutions” or “silent variations,” which are one species of “conservatively modified variations.” Every polynucleotide sequence described herein which encodes a polypeptide also describes every possible silent variation, except where otherwise noted. Thus, silent substitutions are an implied feature of every nucleic acid sequence which encodes an amino acid. One of skill will recognize that each codon in a nucleic acid (except AUG, which is ordinarily the only codon for methionine) can be modified to yield a functionally identical molecule by standard techniques. In some embodiments, the nucleotide sequences that encode the enzymes are preferably optimized for expression in a particular host cell (e.g., yeast, mammalian, plant, fungal, and the like) used to produce the enzymes.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7)Serine (S), Threonine (T); and 8) Cysteine (C), Methionine (M) (see, e.g., Creighton, 1984, PROTEINS).

Macromolecular structures such as polypeptide structures can be described in terms of various levels of organization. For a general discussion of this organization, see, e.g., Alberts et al., 1994, MOLECULAR BIOLOGY OF THE CELL, 3^{rd}, Ed., and Cantor and Schimmel, 1980, BIOPHYSICAL CHEMISTRY Part I: THE CONFORMATION OF BIOLOGICAL MACROMOLECULES. “Primary structure” refers to the amino acid sequence of a particular peptide. “Secondary structure” refers to locally ordered, three dimensional structures within a polypeptide. These structures are commonly known as domains. Domains are portions of a polypeptide that form a compact unit of the polypeptide and are typically 50 to 350 amino acids long. Typical domains are made up of sections of lesser organization such as stretches of β-sheet and α-helices. “Tertiary structure” refers to the complete three dimensional structure of a polypeptide monomer. “Quaternary structure” refers to the three dimensional structure formed by the noncovalent association of independent tertiary units. Anisotropic terms are also known as energy terms.

A particular nucleic acid sequence also implicitly encompasses “splice variants.” Similarly, a particular protein encoded by a nucleic acid implicitly encompasses any protein encoded by a splice variant of that nucleic acid. “Splice variants,” as the name suggests, are products of alternative splicing of a gene. After transcription, an initial nucleic acid transcript may be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition.

“Biological sample,” as used herein, refers to a sample of cells, biological tissue or fluid that contains one or more ZNF217 nucleic acids encoding one or more ZNF217 proteins. Most often, the sample has been removed from a patient or subject, but the term “biological sample” can also refer to cells or tissue analyzed in vivo, i.e., without removal from the patient or subject. Typically, a “biological sample” will contain cells from the patient or subject, but the term can also refer to noncellular biological material, such as noncellular fractions of blood, saliva, or urine, that can be used to measure ZNF217 levels. Numerous types of biological samples can be used in the present invention, including, but not limited to, a tissue biopsy, a blood sample, a buccal scrape, a saliva sample, or a nipple discharge. Such samples include, but are not limited to, tissue isolated from humans, mice, and rats, in particular, breast and lung tissue as well as blood, lymphatic tissue, liver, brain, heart, spleen, testis, ovary, thymus, kidney, and embryonic tissues. Biological samples can also include sections of tissues such as frozen sections taken for histological purposes. A biological sample is typically obtained from a mammal such as rat, mouse, cow, dog, cat, guinea pig, or rabbit, and most preferably a primate such as a chimpanzee or a human.

“Providing a biological sample” means to obtain a biological sample for use in the methods described in this invention. Most often, this will be done by removing a sample of cells from a patient or subject, but can also be accomplished by using previously isolated cells (e.g., isolated by another person, at another time, and/or for another purpose), or by performing the methods of the invention in vivo.

As used herein, a “tissue biopsy” refers to an amount of tissue removed from a patient or subject for diagnostic analysis. In a patient with cancer, tissue can be removed from a tumor, allowing the analysis of cells within the tumor. “Tissue biopsy” can refer to any type of biopsy, such as needle biopsy, fine needle biopsy, surgical biopsy, and the like.

A “control sample” refers to a sample of biological material representative of healthy, cancer-free patients. The level of ZNF217 in a control sample is desirably typical of the general population of normal, cancer-free patients. This sample can be removed from a patient expressly for use in the methods described in this invention, or can be any biological material representative of normal, cancer-free patients. A control sample can also be obtained from normal tissue from the patient that has cancer or is suspected of having cancer. A control sample can also refer to an established level of ZNF217, representative of the cancer-free population, that has been previously established based on measurements from normal, cancer-free patients. If a detection method is used that only detects ZNF217 when a level higher than that typical of a normal, cancer-free patient is present, i.e., an immunohistochemical assay giving a simple positive or negative result, this is considered to be assessing the ZNF217 level in comparison to the control level, as the control level is inherent in the assay.

The “level of ZNF217 mRNA” in a biological sample refers to the amount of mRNA transcribed from a ZNF217 gene that is present in a cell or a biological sample. The mRNA generally encodes a functional ZNF217 protein, although mutations or microdeletions can be present that alter or eliminate the function of the encoded protein. A “level of ZNF217 mRNA” need not be quantified, but can simply be detected, e.g., a subjective, visual detection by a human, with or without comparison to a level from a control sample or a level expected of a control sample.

The “level of ZNF217 protein or polypeptide” in a biological sample refers to the amount of polypeptide translated from a ZNF217 mRNA that is present in a cell or biological sample. The polypeptide can or can not have ZNF217 protein activity. A “level of ZNF217 protein” need not be quantified, but can simply be detected, e.g., a subjective, visual detection by a human, with or without comparison to a level from a control sample or a level expected of a control sample.

An “increased” or “elevated” level of ZNF217 refers to a level of ZNF217 polynucleotide, e.g., genomic DNA, or mRNA, or polypeptide, that, in comparison with a control level of ZNF217, is detectably higher. The method of comparison can be statistical, using quantified values for the level of ZNF217, or can be compared using nonstatistical means, such as by a visual, subjective assessment by a human.

For diagnostic and prognostic applications in cancer, a level of ZNF217 polypeptide or polynucleotide that is “expected” in a control sample refers to a level that represents a typical, cancer-free sample, and from which an elevated, or diagnostic, presence of ZNF217 polypeptide or polynucleotide can be distinguished. Preferably, an “expected” level will be controlled for such factors as the age, sex, and medical history, of the patient or subject, as well as for the particular biological sample being tested.

The phrase “functional effects” in the context of assays for testing compounds that modulate ZNF217 activity includes the determination of any parameter that is indirectly or directly under the influence of ZNF217, e.g., a functional, physical, or chemical effect. These effects include gene amplification, or expression in cancer cells. “Functional effects” include in vitro, in vivo, and ex vivo activities.

By “determining the functional effect” is meant assaying for a compound that increases or decreases a parameter that is indirectly or directly under the influence of ZNF217, e.g., functional, physical and chemical effects. Such functional effects can be measured by any means known to those skilled in the art, e.g., changes in spectroscopic characteristics (e.g., fluorescence, absorbance, refractive index), hydrodynamic (e.g., shape), chromatographic, or solubility properties for the protein, measuring inducible markers or transcriptional activation of ZNF217; or binding assays, e.g., measuring the association of ZNF217 with other proteins.

“Inhibitors” and “modulators” of ZNF217 are used to refer to inhibitory or modulating molecules identified using in vitro and in vivo assays of ZNF217, e.g., ZNF217 expression in cell membranes. Inhibitors are compounds that, e.g., bind to, partially or totally block activity, decrease, prevent, delay activation, inactivate, desensitize, or down regulate the activity of ZNF217, e.g., antagonists. Activators are compounds that, e.g., increase ZNF217 activity, or increase ZNF217 expression or stability. Modulators of ZNF217 also include genetically modified versions of ZNF217, e.g., versions with altered activity, as well as naturally occurring and synthetic ligands, antagonists, agonists, antibodies, siRNAs, small chemical molecules and the like. Assays for inhibitors and activators of ZNF217 include, e.g., expressing ZNF217 in vitro, in cells, or cell membranes, applying putative modulator compounds, and then determining the functional effects on ZNF217 activity, as described above.

Samples or assays comprising ZNF217 polypeptides that are treated with a potential activator, inhibitor, or modulator are compared to control samples without the inhibitor, activator, or modulator to examine the effect of the candidate compound. Control samples (untreated with the compound) are assigned a relative ZNF217 activity value of 100%. Inhibition of a ZNF217 polypeptide is achieved when the activity value relative to the control is about 80%, optionally about 50% or 25-0%. Activation of a ZNF217 polypeptide is achieved when the activity value relative to the control is about 110%, optionally about 150%, optionally about 200-500%, or about 1000-3000% higher.

The terms “isolated”, “purified”, or “biologically pure” refer to material that is substantially or essentially free from components which normally accompany it as found in its native state. Purity and homogeneity are typically determined using analytical chemistry techniques such as polyacrylamide gel electrophoresis or high performance liquid chromatography. A protein that is the predominant species present in a preparation is substantially purified. In particular, an isolated ZNF217 nucleic acid is separated from open reading frames that flank the ZNF217 gene and encode proteins other than ZNF217. The term “purified” denotes that a nucleic acid or protein gives rise to essentially one band in an electrophoretic gel. Particularly, it means that the nucleic acid or protein is at least 85% pure, optionally at least 95% pure, and optionally at least 99% pure.

“Nucleic acid” refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions can be achieved by generating sequences in which the third position of one or more selected (or all) codons is substituted with mixed-base and/or deoxyinosine residues (Batzer et al., 1991, Nucleic Acid Res. 19:5081; Ohtsuka et al., 1985, J. Biol. Chem. 260:2605-2608; Rossolini et al., 1994, Mol. Cell. Probes 8:91-98). The term nucleic acid is used interchangeably with gene, cDNA, mRNA, oligonucleotide, and polynucleotide.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid.

Amino acids can be referred to herein by either their commonly known three letter symbols or by the one-letter symbols recommended by the IUPAC-IUB Biochemical Nomenclature Commission. Nucleotides, likewise, can be referred to by their commonly accepted single-letter codes.

A “label” or a “detectable moiety” is a composition detectable by spectroscopic, photochemical, biochemical, immunochemical, or chemical means. For example, useful labels include ³²p, fluorescent dyes, electron-dense reagents, enzymes (e.g., as commonly used in an ELISA), biotin, digoxigenin, or haptens and proteins which can be made detectable, e.g., by incorporating a radiolabel into the peptide or used to detect antibodies specifically reactive with the peptide.

A “labeled nucleic acid probe or oligonucleotide” is one that is bound, either covalently, through a linker or a chemical bond, or noncovalently, through ionic, van der Waals, electrostatic, or hydrogen bonds to a label such that the presence of the probe can be detected by detecting the presence of the label bound to the probe.

As used herein a “nucleic acid probe or oligonucleotide” is defined as a nucleic acid capable of binding to a target nucleic acid of complementary sequence through one or more types of chemical bonds, usually through complementary base pairing, usually through hydrogen bond formation. As used herein, a probe can include natural (i.e., A, G, C, or T) or modified bases (7-deazaguanosine, inosine, and the like). In addition, the bases in a probe can be joined by a linkage other than a phosphodiester bond, so long as it does not interfere with hybridization. Thus, for example, probes can be peptide nucleic acids in which the constituent bases are joined by peptide bonds rather than phosphodiester linkages. It will be understood by one of skill in the art that probes can bind target sequences lacking complete complementarity with the probe sequence depending upon the stringency of the hybridization conditions. The probes are optionally directly labeled as with isotopes, chromophores, lumiphores, chromogens, or indirectly labeled such as with biotin to which a streptavidin complex can later bind. By assaying for the presence or absence of the probe, one can detect the presence or absence of the select sequence or subsequence.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (nonrecombinant) form of the cell or express native genes that are otherwise abnormally expressed, under expressed or not expressed at all.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

A “promoter” is defined as an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. A “constitutive” promoter is a promoter that is active under most environmental and developmental conditions. An “inducible” promoter is a promoter that is active under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

An “expression vector” is a nucleic acid construct, generated recombinantly or synthetically, with a series of specified nucleic acid elements that permit transcription of a particular nucleic acid in a host cell. The expression vector can be part of a plasmid, virus, or nucleic acid fragment. Typically, the expression vector includes a nucleic acid to be transcribed operably linked to a promoter.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 70% identity, preferably 75%, 80%, 85%, 86%, 87%, 88%, 89%, 90%, 91%, 92%, 93%, 94%, 95%, 96%, 97%, 98%, 99%, or higher identity over a specified region (e.g., the sequence of the naturally occurring ZFN217 gene), when compared and aligned for maximum correspondence over a comparison window or designated region) as measured using a BLAST or BLAST 2.0 sequence comparison algorithms with default parameters described below, or by manual alignment and visual inspection. Such sequences are then said to be “substantially identical.” This definition also refers to the compliment of a test sequence. The definition also includes sequences that have deletions and/or additions, as well as those that have substitutions. As described below, the preferred algorithms can account for gaps and the like. Preferably, the identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence can be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, 1991, Adv. Appl. Math. 2:482, by the homology alignment algorithm of Needleman & Wunsch, 1970, J. Mol. Biol. 48:443, by the search for similarity method of Pearson & Lipman, 1988, Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., CURRENT PROTOCOLS IN MOLECULAR BIOLOGY (Ausubel et al., eds. 1995 supplement).

Another example of algorithm that is suitable for determining percent sequence identity and sequence similarity are the BLAST and BLAST 2.0 algorithms, which are described in Altschul et al., 1977, Nuc. Acids Res. 25:3389-3402 and Altschul et al., 1990, J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, 1989, Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin & Altschul, 1993, Proc. Nat'l. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA).

The phrase “stringent hybridization conditions” refers to conditions under which a probe will hybridize to its target subsequence, typically in a complex mixture of nucleic acid, but to no other sequences. Stringent conditions are sequence-dependent and will be different in different circumstances. Longer sequences hybridize specifically at higher temperatures. An extensive guide to the hybridization of nucleic acids is found in Tijssen, 1993, “Overview of principles of hybridization and the strategy of nucleic acid assays” in TECHNIQUES IN BIOCHEMISTRY AND MOLECULAR BIOLOGY—HYBRIDIZATION WITH NUCLEIC PROBES. Generally, stringent conditions are selected to be about 5-10° C. lower than the thermal melting point (T_{M}) for the specific sequence at a defined ionic strength pH. The T_{M} is the temperature (under defined ionic strength, pH, and nucleic concentration) at which 50% of the probes complementary to the target hybridize to the target sequence at equilibrium (as the target sequences are present in excess, at T_{M}, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.5 and the temperature is at least about 30° C. for short probes (e.g., 10 to 50 nucleotides) and at least about 60° C. for long probes (e.g., greater than 50 nucleotides). Stringent conditions can also be achieved with the addition of destabilizing agents such as formamide. For selective or specific hybridization, a positive signal is at least two times background, optionally 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C. Such washes can be performed for 5, 15, 30, 60, 120, or more minutes. For PCR, a temperature of about 36° C. is typical for low stringency amplification, although annealing temperatures can vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec -2 min., an annealing phase lasting 30 sec.-2 min., and an extension phase of about 72° C. for 1-2 min.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. Such washes can be performed for 5, 15, 30, 60, 120, or more minutes. A positive hybridization is at least twice background. Those of ordinary skill will readily recognize that alternative hybridization and wash conditions can be utilized to provide conditions of similar stringency.

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′2, a dimer of Fab which itself is a light chain joined to V_{H}-C_{H1} by a disulfide bond. The F(ab)′2 can be reduced under mild conditions to break the disulfide linkage in the hinge region, thereby converting the F(ab)′2 dimer into an Fab′ monomer. The Fab′ monomer is essentially Fab with part of the hinge region (see FUNDAMENTAL IMMUNOLOGY (Paul Ed., 3^{rd} Ed. 1993). While various antibody fragments are defined in terms of the digestion of an intact antibody, one of skill will appreciate that such fragments can be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., 1990, Nature 348:552-554).

For preparation of monoclonal or polyclonal antibodies, any technique known in the art can be used (see, e.g., Kohler & Milstein, 1975, Nature 256:495-497; Kozbor et al., 1983, Immunology Today 4:72; Cole et al., 1985, pp. 77-96 in MONOCLONAL ANTIBODIES AND CANCER THERAPY). Techniques for the production of single chain antibodies (U.S. Pat. No. 4,946,778) can be adapted to produce antibodies to polypeptides of this invention. Also, transgenic mice, or other organisms such as other mammals, can be used to express humanized antibodies. Alternatively, phage display technology can be used to identify antibodies and heteromeric Fab fragments that specifically bind to selected antigens (see, e.g., McCafferty et al., 1990, Nature 348:552-554; Marks et al., 1992, Biotechnology 10:779-783).

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

An “anti-ZNF217” antibody is an antibody or antibody fragment that specifically binds a polypeptide encoded by a ZNF217 gene, cDNA, or a subsequence thereof, e.g., the C-terminal domain.

The term “immunoassay” is an assay that uses an antibody to specifically bind an antigen. The immunoassay is characterized by the use of specific binding properties of a particular antibody to isolate, target, and/or quantify the antigen.

The phrase “specifically (or selectively) binds” to an antibody or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions can require an antibody that is selected for its specificity for a particular protein. For example, polyclonal antibodies raised to a ZNF217 polypeptide from specific species such as rat, mouse, or human can be selected to obtain only those polyclonal antibodies that are specifically immunoreactive with the ZNF217 protein and not with other proteins, except for polymorphic variants and alleles of the ZNF217 protein. This selection can be achieved by subtracting out antibodies that cross-react with ZNF217 molecules from other species. A variety of immunoassay formats can be used to select antibodies specifically immunoreactive with a particular protein. For example, solid-phase ELISA immunoassays are routinely used to select antibodies specifically immunoreactive with a protein (see, e.g., Harlow & Lane, 1988, ANTIBODIES, A LABORATORY MANUAL, for a description of immunoassay formats and conditions that can be used to determine specific immunoreactivity). Typically a specific or selective reaction will be at least twice background signal or noise and more typically more than 10 to 100 times background.

The phrase “selectively associates with” refers to the ability of a nucleic acid to “selectively hybridize” with another as defined above, or the ability of an antibody to “selectively (or specifically) bind” to a protein, as defined above.

By “host cell” is meant a cell that contains an expression vector and supports the replication or expression of the expression vector. Host cells can be prokaryotic cells such as E. coli, or eukaryotic cells such as yeast, insect, amphibian, or mammalian cells such as CHO, HeLa and the like, e.g., cultured cells, explants, and cells in vivo.

The phrase “detecting a cancer” refers to the ascertainment of the presence or absence of cancer in patient. “Detecting a cancer” can also refer to obtaining indirect evidence regarding the likelihood of the presence of cancerous cells in the patient. Detecting a cancer can be accomplished using the methods of this invention alone, in combination with other methods, or in light of other information regarding the state of health of the patient or subject.

“Providing a biological sample” means to obtain a biological sample for use in the methods described in this invention. Most often, this will be done by removing a sample of cells from a patient or subject, but can also be accomplished by using previously isolated cells (e.g., isolated by another person, at another time, and/or for another purpose), or by performing the methods of the invention in vivo.

A “control sample” refers to a sample of biological material representative of healthy, cancer-free patients. The level of ZNF217 in a control sample is desirably typical of the general population of normal, cancer-free patients. This sample can be removed from a patient or subject expressly for use in the methods described in this invention, or can be any biological material representative of normal, cancer-free patiens. A control sample can also refer to an established level of ZNF217, representative of the cancer-free population, that has been previously established based on measurements from normal, cancer-free patients. If a detection method is used that only detects ZNF217 when a level higher than that typical of a normal, cancer-free animal is present, i.e., an immunohistochemical assay giving a simple positive or negative result, this is considered to be assessing the ZNF217 level in comparison to the control level, as the control level is inherent in the assay.

By “therapeutically effective dose” herein is meant a dose that produces effects for which it is administered. The exact dose will depend on the purpose of the treatment, and will be ascertainable by one skilled in the art using known techniques (see, e.g., Lieberman, PHARMACEUTICAL DOSAGE FORMS (Vols. 1-3, 1992); Lloyd, 1999, THE ART, SCIENCE AND TECHNOLOGY OF PHARMACEUTICAL COMPOUNDING; and Pickar, 1999, DOSAGE CALCULATIONS).

III. Detection of ZNF217 Nucleic Acids

In numerous embodiments of the present invention, nucleic acids encoding a ZNF217 polypeptide, including a full-length ZNF217 protein, or any derivative, variant, homolog, or fragment thereof, will be used. Such nucleic acids are useful for any of a number of applications, including for the production of ZNF217 protein, for diagnostic assays, for therapeutic applications, for ZNF217-specific probes, for assays for ZNF217 binding and/or modulating compounds, to identify and/or isolate ZNF217 homologs from other species or from mice, and other applications.

A. General Recombinant DNA Methods

Numerous applications of the present invention involve the cloning, synthesis, maintenance, mutagenesis, and other manipulations of nucleic acid sequences that can be performed using routine techniques in the field of recombinant genetics. Basic texts disclosing the general methods of use in this invention include Sambrook et al., MOLECULAR CLONING, A LABORATORY MANUAL (2^{nd.} Ed. 1989); Kriegler, 1990, GENE TRANSFER AND EXPRESSION:A LABORATORY MANUAL; and CURRENT PROTOCOLS IN MOLECULAR BIOLOGY, 1995, (Ausubel et al., eds.).

For nucleic acids, sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kDa) or amino acid residue numbers. Proteins sizes are estimated from gel electrophoresis, from sequenced proteins, from derived amino acid sequences, or from published protein sequences.

Oligonucleotides that are not commercially available can be chemically synthesized according to the solid phase phosphoramidite triester method first described by Beaucage & Caruthers, 1981, Tetrahedron Letts. 22:1859-1862, using an automated synthesizer, as described in Van Devanter et al., 1984, Nucleic Acids Res. 12:6159-6168. Purification of oligonucleotides is by either native acrylamide gel electrophoresis or by anion-exchange HPLC as described in Pearson & Reanier, 1983, J. Chrom. 255:137-149.

The sequence of the cloned genes and synthetic oligonucleotides can be verified after cloning using, e.g., the chain termination method for sequencing double-stranded templates of Wallace et al., 1981, Gene 16:21-26.

B. Isolating and Detecting ZNF217 Nucleotide Sequences

In numerous embodiments of the present invention, ZNF217 nucleic acids will be isolated and cloned using recombinant methods. Such embodiments are used, e.g., to isolate ZNF217 polynucleotides for protein expression or during the generation of variants, derivatives, expression cassettes, or other sequences derived from ZNF217, to monitor ZNF217 gene expression, for the determination of ZNF217 sequences in various species, for diagnostic purposes in a patient, i.e., to detect mutations in ZNF217, or for genotyping and/or forensic applications.

Polymorphic variants, alleles, and interspecies homologs and nucleic acids that are substantially identical to the ZNF217 gene can be isolated using ZNF217 nucleic acid probes, and oligonucleotides by screening libraries under stringent hybridization conditions. Alternatively, expression libraries can be used to clone ZNF217 protein, polymorphic variants, alleles, and interspecies homologs, by detecting expressed homologs immunologically with antisera or purified antibodies made against a ZNF217 polypeptide, which also recognize and selectively bind to the ZNF217 homolog.

To make a cDNA library, one should choose a source that is rich in ZFN217 RNA. The mRNA is then made into cDNA using reverse transcriptase, ligated into a recombinant vector, and transfected into a recombinant host for propagation, screening and cloning. Methods for making and screening cDNA libraries are well known (see, e.g., Gubler & Hoffman, 1983, Gene 25:263-269; Sambrook et al., supra; Ausubel et al., supra).

For a genomic library, the DNA is extracted from the tissue and either mechanically sheared or enzymatically digested to yield fragments of about 12-20 kb. The fragments are then separated by gradient centrifugation from undesired sizes and are constructed in bacteriophage lambda vectors. These vectors and phage are packaged in vitro. Recombinant phage are analyzed by plaque hybridization as described in Benton & Davis, 1977, Science 196:180-182. Colony hybridization is carried out as generally described in Grunstein et al., 1975, Proc. Natl. Acad. Sci. USA., 72:3961-3965.

More distantly related ZNF217 homologs can be identified using any of a number of well known techniques, including by hybridizing a ZNF217 probe with a genomic or cDNA library using moderately stringent conditions, or under low stringency conditions using probes from regions which are selective for ZNF217, e.g., specific probes generated to the C-terminal domain. Also, a distant homolog can be amplified from a nucleic acid library using degenerate primer sets, i.e., primers that incorporate all possible codons encoding a given amino acid sequence, in particular based on a highly conserved amino acid stretch. Such primers are well known to those of skill, and numerous programs are available, e.g., on the internet, for degenerate primer design.

In certain embodiments, ZNF217 polynucleotides will be detected using hybridization-based methods to determine, e.g., ZNF217 RNA levels or to detect particular DNA sequences, e.g., for diagnostic purposes. For example, gene expression of ZNF217 can be analyzed by techniques known in the art, e.g., Northern blotting, reverse transcription and PCR amplification of mRNA, including quantitative PCR analysis of mRNA levels with real-time PCR procedures (e.g., reverse transcriptase-TAQMAN™ amplification), dot blotting, in situ hybridization, RNase protection, probing DNA microchip arrays, and the like.

In one embodiment, high density oligonucleotide analysis technology (e.g., GeneChip™) is used to identify orthologs, alleles, conservatively modified variants, and polymorphic variants of ZNF217, or to monitor levels of ZNF217 mRNA. In the case where a homologs is linked to a known disease, they can be used with GeneChip™ as a diagnostic tool in detecting the disease in a biological sample, see, e.g., Gunthand et al., 1998, AIDS Res. Hum. Retroviruses 14:869-876; Kozal et al., 1996, Nat. Med. 2:753-759; Matson et al., 1995, Anal. Biochem. 224:110-106; Lockhart et al., 1996, Nat. Biotechnol. 14:1675-1680; Gingeras et al., 1998, Genome Res. 8:435-448; Hacia et al., 1998, Nucleic Acids Res. 26:3865-3866.

Detection of ZNF217 polynucleotides and polypeptides can involve quantitative or qualitative detection of the polypeptide or polynucleotide, and can involve an actual comparison with a control value or, alternatively, can be performed so that the detection itself inherently indicates an increased level of ZNF217. The ZFN217 nucleic acids, polymorphic variants, orthologs, and alleles can modulate the expression, stability or activity of the naturally occurring ZNF217 gene or other ZNF217 family members, such that women with increased levels of protein have an increased risk of cancer, e.g., breast cancer, discussed infra.

In certain embodiments, for example, diagnosis of cancer, the level of ZNF217 polynucleotide, polypeptide, or protein activity will be quantified. In such embodiments, the difference between an elevated level of ZNF217 and a normal, control level will preferably be statistically significant. Typically, a diagnostic presence, i.e., overexpression or an increase of ZNF217 polypeptide or nucleic acid, represents at least about a 1.5, 2, 3, 5, 10, or greater fold increase in the level of ZNF217 polypeptide or polynucleotide in the biological sample compared to a level expected in a noncancerous sample. Detection of ZNF217 can be performed in vitro, i.e., in cells within a biological sample taken from the patient, or in vivo. In one embodiment an increased level of ZNF217 is used as a diagnostic marker of ZNF217. As used herein, a “diagnostic presence” indicates any level of ZNF217 that is greater than that expected in a noncancerous sample. In a one embodiment, assays for a ZNF217 polypeptide or polynucleotide in a biological sample are conducted under conditions wherein a normal level of ZNF217 polypeptide or polynucleotide, i.e., a level typical of a noncancerous sample, i.e., cancer-free, would not be detected. In such assays, therefore, the detection of any ZNF217 polypeptide or nucleic acid in the biological sample indicates a diagnostic presence, or increased level.

As described below, any of a number of methods to detect ZNF217 can be used. A ZNF217 polynucleotide level can be detected by detecting any ZNF217 DNA or RNA, including ZNF217 genomic DNA, mRNA, and cDNA. A ZNF217 polypeptide can be detected by detecting a ZNF217 polypeptide itself, or by detecting ZNF217 protein activity. Detection can involve quantification of the level of ZNF217 (e.g., genomic DNA, cDNA, mRNA, or protein level, or protein activity) or, alternatively, can be a qualitative assessment of the level, or of the presence or absence, of ZNF217, in particular in comparison with a control level. Any of a number of methods to detect any of the above can be used, as described infra. Such methods include, for example, hybridization, amplification, and other assays.

In certain embodiments, the ability to detect an increased level, or diagnostic presence, in a cell is used as a marker for cancer cells, i.e., to monitor the number or localization of cancer cells in a patient, as detected in vivo or in vitro.

Typically, the ZNF217 polynucleotides or polypeptides detected herein will be at least about 70% identical, and preferably 75%, 80%, 85%, 86%, 87%, 88%, 89%, 90%, 91%, 92%, 93%, 94%, 95%, 96%, 97%, 98%, 99% or more identical, over a region of at least about 50, 100, 200, or more nucleotides, or 20, 50, 100, or more amino acids, to the naturally occurring ZNF217 gene. Such polynucleotides or polypeptides can represent functional or nonfunctional forms of ZNF217, or any variant, derivative, or fragment thereof.

1. Detection of Copy Number

In one embodiment, e.g., for the diagnosis or presence of cancer, the copy number, i.e., the number of ZNF217 genes in a cell, is evaluated. Generally, for a given autosomal gene, an animal has two copies of each gene. The copy number can be increased, however, by gene amplification or duplication, e.g., in cancer cells, or reduced by deletion. Methods of evaluating the copy number of a particular gene are well known to those of skill in the art, and include, inter alia, hybridization- and amplification-based assays.

a) Hybridization-based Assays

Any of a number of hybridization-based assays can be used to detect the ZNF217 gene or the copy number of ZNF217 genes in the cells of a biological sample. One such method is by Southern blot. In a Southern blot, genomic DNA is typically fragmented, separated electrophoretically, transferred to a membrane, and subsequently hybridized to a ZNF217-specific probe. For copy number determination, comparison of the intensity of the hybridization signal from the probe for the target region with a signal from a control probe for a region of normal genomic DNA (e.g., a nonamplified portion of the same or related cell, tissue, organ, and the like) provides an estimate of the relative ZNF217 copy number. Southern blot methodology is well known in the art and is described, e.g., in Ausubel et al., or Sambrook et al., supra.

An alternative means for determining the copy number of ZNF217 genes in a sample is by in situ hybridization, e.g., fluorescence in situ hybridization, or FISH. In situ hybridization assays are well known (e.g., Angerer, 1987, Meth. Enzymol 152:649). Generally, in situ hybridization comprises the following major steps:(1) fixation of tissue or biological structure to be analyzed; (2) prehybridization treatment of the biological structure to increase accessibility of target DNA, and to reduce nonspecific binding; (3) hybridization of the mixture of nucleic acids to the nucleic acid in the biological structure or tissue; (4) post-hybridization washes to remove nucleic acid fragments not bound in the hybridization; and (5) detection of the hybridized nucleic acid fragments.

The probes used in such applications are typically labeled, e.g., with radioisotopes or fluorescent reporters. Preferred probes are sufficiently long, e.g., from about 50, 100, or 200 nucleotides to about 1000 or more nucleotides, so as to specifically hybridize with the target nucleic acid(s) under stringent conditions.

In numerous embodiments “comparative probe” methods, such as comparative genomic hybridization (CGH), are used to detect ZNF217 gene amplification. In comparative genomic hybridization methods, a “test” collection of nucleic acids is labeled with a first label, while a second collection (e.g., from a healthy cell or tissue) is labeled with a second label. The ratio of hybridization of the nucleic acids is determined by the ratio of the first and second labels binding to each fiber in an array. Differences in the ratio of the signals from the two labels, e.g., due to gene amplification in the test collection, is detected and the ratio provides a measure of the ZNF217 gene copy number.

Hybridization protocols suitable for use with the methods of the invention are described, e.g., in Albertson, 1984, EMBO J. 3:1227-1234; Pinkel, 1988, Proc. Natl. Acad. Sci. USA 85:9138-9142; EPO Pub. No. 430,402; METHODS IN MOLECULAR BIOLOGY, VOL. 33: In Situ Hybridization Protocols, Choo, Ed., 1994, Humana Press, Totowa, N.J., and the like.

b) Amplification-based Assays

In another embodiment, amplification-based assays are used to detect ZNF217 or to measure the copy number of ZNF217 genes. In such assays, the ZNF217 nucleic acid sequences act as a template in an amplification reaction (e.g., Polymerase Chain Reaction, or PCR). In a quantitative amplification, the amount of amplification product will be proportional to the amount of template in the original sample. Comparison to appropriate controls provides a measure of the copy number of the ZNF217 gene. Methods of quantitative amplification are well known to those of skill in the art. Detailed protocols for quantitative PCR are provided, e.g., in Innis et al., 1990, PCR PROTOCOLS: A GUIDE TO METHODS AND APPLICATIONS, Academic Press, Inc. N.Y.). The nucleic acid sequence for ZNF217 is sufficient to enable one of skill to routinely select primers to amplify any portion of the gene.

In some embodiments, a TaqMan based assay is used to quantify ZNF217 polynucleotides. TaqMan based assays use a fluorogenic oligonucleotide probe that contains a 5′ fluorescent dye and a 3′ quenching agent. The probe hybridizes to a PCR product, but cannot itself be extended due to a blocking agent at the 3′ end. When the PCR product is amplified in subsequent cycles, the 5′ nuclease activity of the polymerase, e.g., AmpliTaq, results in the cleavage of the TaqMan probe. This cleavage separates the 5′ fluorescent dye and the 3′ quenching agent, thereby resulting in an increase in fluorescence as a function of amplification (see, for example, literature provided by Perkin-Elmer, e.g., www.perkin-elmer.com).

Other suitable amplification methods include, but are not limited to, ligase chain reaction (LCR) (see, Wu and Wallace, 1989, Genomics 4:560, Landegren et al., 1988, Science 241:1077, and Barringer et al., 1990, Gene 89:117), transcription amplification (Kwoh et al., 1989, Proc. Natl. Acad. Sci. USA 86:1173), self-sustained sequence replication (Guatelli et al., 1990, Proc. Nat. Acad. Sci. USA 87:1874), dot PCR, and linker adapter PCR, etc.

2. Detection of ZNF217 Expression

a) Direct hybridization-based assays

Methods of detecting and/or quantifying the level of ZNF217 gene transcripts (mRNA or cDNA made therefrom) using nucleic acid hybridization techniques are known to those of skill in the art (see, Sambrook et al., 1989, MOLECULAR CLONING: A LABORATORY MANUAL, 2D ED., VOLS 1-3, Cold Spring Harbor Press, New York).

For example, one method for evaluating the presence, absence, or quantity of ZNF217 cDNA involves a Northern blot. In brief, in a typical embodiment, mRNA is isolated from a given biological sample, electrophoresed to separate the mRNA species, and transferred from the gel to a nitrocellulose membrane. Labeled ZNF217 probes are then hybridized to the membrane to identify and/or quantify the mRNA.

b) Amplification-based assays

In another embodiment, a ZNF217 transcript (e.g., ZNF217 mRNA) is detected using amplification-based methods (e.g., RT-PCR). RT-PCR methods are well known to those of skill (see, e.g., Ausubel et al., supra). Preferably, quantitative RT-PCR is used, thereby allowing the comparison of the level of mRNA in a sample with a control sample or value.

3. Detection of ZNF217 Polypeptide Expression

In addition to the detection of ZNF217 genes and gene expression using nucleic acid hybridization technology, ZNF217 levels can also be detected and/or quantified by detecting or quantifying the polypeptide. ZNF217 polypeptides are detected and quantified by any of a number of means well known to those of skill in the art. These include analytic biochemical methods such as electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunoelectrophoresis, radioimmunoassay (RIA), enzyme-linked immunosorbent assays (ELISAs), immunofluorescent assays, western blotting, and the like. ZNF217 polypeptide detection is discussed in Section VI, infra.

C. Expression in Prokaryotes and Eukaryotes

In some embodiments, it is desirable to produce ZNF217 polypeptides using recombinant technology. To obtain high level expression of a cloned gene or nucleic acid, such as a cDNA encoding a ZNF217 polypeptide, a ZNF217 sequence is typically subcloned into an expression vector that contains a strong promoter to direct transcription, a transcription/translation terminator, and if for a nucleic acid encoding a protein, a ribosome binding site for translational initiation. Suitable bacterial promoters are well known in the art and are described, e.g., in Sambrook et al. and Ausubel et al. Bacterial expression systems for expressing the ZNF217 protein are available in, e.g., E. coli, Bacillus sp., and Salmonella (Palva et al., 1983, Gene 22:229-235; Mosbach et al., 1983, Nature 302:543-545. Kits for such expression systems are commercially available. Eukaryotic expression systems for mammalian cells, yeast, and insect cells are well known in the art and are also commercially available. In one embodiment, the eukaryotic expression vector is an adenoviral vector, an adeno-associated vector, or a retroviral vector.

For therapeutic applications, ZNF217 nucleic acids are introduced into a cell, in vitro, in vivo, or ex vivo, using any of a large number of methods including, but not limited to, infection with viral vectors, liposome-based methods, biolistic particle acceleration (the gene gun), and naked DNA injection. Such therapeutically useful nucleic acids include, but are not limited to, coding sequences for full-length ZNF217, coding sequences for a ZNF217 fragment, domain, derivative, or variant, ZNF217 antisense sequences, ZNF217 siRNA sequences, and ZNF217 ribozymes. Typically, such sequences will be operably linked to a promoter, but in numerous applications a nucleic acid will be administered to a cell that is itself directly therapeutically effective, e.g., certain antisense, siRNA, or ribozyme molecules.

The promoter used to direct expression of a heterologous nucleic acid depends on the particular application. The promoter is optionally positioned about the same distance from the heterologous transcription start site as it is from the transcription start site in its natural setting. As is known in the art, however, some variation in this distance can be accommodated without loss of promoter function.

In addition to the promoter, the expression vector typically contains a transcription unit or expression cassette that contains all the additional elements required for the expression of the ZNF217-encoding nucleic acid in host cells. A typical expression cassette thus contains a promoter operably linked to the nucleic acid sequence encoding a ZNF217 polypeptide, and signals required for efficient polyadenylation of the transcript, ribosome binding sites, and translation termination. The nucleic acid sequence encoding a ZNF217 polypeptide can be linked to a cleavable signal peptide sequence to promote secretion of the encoded protein by the transfected cell. Such signal peptides would include, among others, the signal peptides from tissue plasminogen activator, insulin, and neuron growth factor, and juvenile hormone esterase of Heliothis virescens. Additional elements of the cassette can include enhancers and, if genomic DNA is used as the structural gene, introns with functional splice donor and acceptor sites.

In addition to a promoter sequence, the expression cassette should also contain a transcription termination region downstream of the structural gene to provide for efficient termination. The termination region can be obtained from the same gene as the promoter sequence or can be obtained from different genes.

The particular expression vector used to transport the genetic information into the cell is not particularly critical. Any of the conventional vectors used for expression in eukaryotic or prokaryotic cells can be used. Standard bacterial expression vectors include plasmids such as pBR322 based plasmids, pSKF, pET23D, and fusion expression systems such as GST and LacZ. Epitope tags can also be added to recombinant proteins to provide convenient methods of isolation, e.g., c-myc, HA-tag, 6-His (SEQ ID NO:1) tag, maltose binding protein, VSV-G tag, anti-DYKDDDDK (SEQ ID NO:2) tag, or any such tag, a large number of which are well known to those of skill in the art.

Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A+, pMTO10/A+, pMAMneo-5, baculovirus pDSVE, and any other vector allowing expression of proteins under the direction of the CMV promoter, SV40 early promoter, SV40 later promoter, metallothionein promoter, murine mammary tumor virus promoter, Rous sarcoma virus promoter, polyhedrin promoter, or other promoters shown effective for expression in eukaryotic cells.

Some expression systems have markers that provide gene amplification, such as neomycin, thymidine kinase, hygromycin B phosphotransferase, and dihydrofolate reductase. Alternatively, high yield expression systems not involving gene amplification are also suitable, such as using a baculovirus vector in insect cells, with a sequence encoding a ZNF217 polypeptide under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable. The prokaryotic sequences are optionally chosen such that they do not interfere with the replication of the DNA in eukaryotic cells, if necessary.

Standard transfection methods are used to produce bacterial, mammalian, yeast or insect cell lines that express large quantities of a ZNF217 protein, which are then purified using standard techniques (see, e.g., Colley et al., 1989, J. Biol. Chem. 264:17619-17622; “Guide to Protein Purification,” in METHODS IN ENZYMOLOGY, Vol. 182, 1990 (Deutscher, Ed.). Transformation of eukaryotic and prokaryotic cells are performed according to standard techniques (see, e.g., Morrison, 1977, J. Bact. 132:349-351; Clark-Curtiss & Curtiss, Methods in Enzymology 101:347-362, 1983 (Wu et al., eds.).

Any of the well known procedures for introducing foreign nucleotide sequences into host cells can be used. These include the use of reagents such as Superfect (Qiagen), liposomes, calcium phosphate transfection, polybrene, protoplast fusion, electroporation, microinjection, plasmid vectors, viral vectors, biolistic particle acceleration (the gene gun), or any of the other well known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing a ZNF217 gene.

After the expression vector is introduced into the cells, the transfected cells are cultured under conditions favoring expression of the ZNF217 polypeptide, which is recovered from the culture using standard techniques identified below. Methods of culturing prokaryotic or eukaryotic cells are well known and are taught, e.g., in Ausubel et al., Sambrook et al., and in Freshney, 1993, CULTURE OF ANIMAL CELLS, 3^{rd.} Ed., A Wiley-Liss Publication.

Any of the well known procedures for introducing foreign nucleotide sequences into host cells can be used to introduce a vector, e.g., a targeting vector, into cells. These include the use of reagents such as Superfect (Qiagen), liposomes, calcium phosphate transfection, polybrene, protoplast fusion, electroporation, microinjection, plasmid vectors, viral vectors, biolistic particle acceleration (the gene gun), or any of the other well known methods for introducing cloned genomic DNA, cDNA, synthetic DNA or other foreign genetic material into a host cell (see, e.g., Sambrook et al., supra). For the generation of a transgenic cell, it is only necessary that the particular genetic engineering procedure used be capable of successfully introducing at least one transgene into at least one host cell, which can then be selected using standard methods. Methods of culturing prokaryotic or eukaryotic cells are well known and are taught, e.g., in Ausubel et al., Sambrook et al., 1993, and in Freshney, CULTURE OF ANIMAL CELLS, 3^{rd.} Ed., A Wiley-Liss Publication.

D. ZNF217 Trangenic Animals

The present invention provides transgenic and chimeric nonhuman mammals comprising one or more functionally and structurally disrupted ZNF217 alleles. A “chimeric animal” includes some cells that lack the functional ZNF217 gene of interest and other cells that do not have the inactivated gene. A “transgenic animal,” in contrast, is made up of cells that have all incorporated the specific modification which renders the ZNF217 gene inactive or otherwise altered. While a transgenic animal is typically always capable of transmitting the mutant ZNF217 gene to its progeny, the ability of a chimeric animal to transmit the mutation depends upon whether the inactivated gene is present in the animal's germ cells. The modifications that inactivate or otherwise alter the ZNF217 gene can include, for example, insertions, deletions, or substitutions of one or more nucleotides. The modifications can interfere with transcription of the gene itself, with translation and/or stability of the resulting mRNA, or can cause the gene to encode an inactive or otherwise altered ZNF217 polypeptide, e.g., a ZNF217 polypeptide with modified binding properties. In particular, the present transgenic and chimeric animals can lack coding sequences for one or more components of a ZNF217 polypeptide, such as the one or more zinc finger binding domains, heterologous protein binding domains. Such transgenes can thus eliminate any one or more codons within an endogenous ZNF217 allele. In a preferred embodiment, a transgenic animal has an allele that lacks at least 10, 20, 30, or more codons of the full-length protein. Further, a transgenic animal can lack non-coding sequences that are required for ZNF217 expression or function, such as 5′ or 3′ regulatory sequences.

Trangenic animals and cells derived from these animals can be used to test compounds as modulators of a ZNF217 protein screening and testing assays described below. In this regard, transgenic animals and cells lines capable of expressing wildtype or mutant ZNF217 can be exposed to test agents. These test agents can be screened for the ability to reduce overexpression of wildtype ZNF217 or impair the expression or function of mutant ZNF217.

Methods of obtaining transgenic animals are described in, for example, PCT Publication No. WO 01/30798, Puhler, A., Ed., 1993, GENETIC ENGINEERING OF ANIMALS, VCH Publ.; Murphy and Carter, eds., 1993, TRANSGENESIS TECHNIQUES: PRINCIPLES AND PROTOCOLS (Methods in Molecular Biology, Vol. 18); and Pinkert, CA, Ed., TRANSGENIC ANIMAL TECHNOLOGY:A LABORATORY HANDBOOK, 1994, Academic Press.

Typically, a modified ZNF217 gene is introduced, e.g., by homologous recombination, into embryonic stem cells (ES), which are obtained from preimplantation embryos and cultured in vitro. See, e.g., Hooper, ML, 1993, EMBRYONAL STEM CELLS: INTRODUCING PLANNED CHANGES INTO THE ANIMAL GERMLINE (Modem Genetics, Vol. 1), Int'l. Pub. Distrib., Inc.; Bradley et al., 1984, Nature, 309:255-258. Subsequently, the transformed ES cell is combined with a blastocyst from a nonhuman animal, e.g., a mouse. The ES cells colonize the embryo and in some embryos form the germ line of the resulting chimeric animal. See, Jaenisch, 1988,Science, 240:1468-1474. Alternatively, ES cells or somatic cells that can reconstitute an organism (“somatic repopulating cells”) can be used as a source of nuclei for transplantation into an enucleated fertilized oocyte giving rise to a transgenic mammal. See, e.g., Wilmut et al., 1997, Nature, 385:810-813.

Other methods for obtaining a transgenic or chimeric animal having a mutant ZNF217 gene in its genome is to contact fertilized oocytes with a vector that includes a polynucleotide that encodes a modified, e.g., inactive, ZNF217 polypeptide. In some animals, such as mice, fertilization is typically performed in vivo and fertilized ova are surgically removed. In other animals, particularly bovines, it is preferable to remove ova from live or slaughterhouse animals and fertilize the ova in vitro. See, DeBoer et al., WO 91/08216. In vitro fertilization permits the modifications to be introduced into substantially synchronous cells.

Fertilized oocytes are typically cultured in vitro until a pre-implantation embryo is obtained containing about 16-150 cells. The 16-32 cell stage of an embryo is described as a morula, whereas pre-implantation embryos containing more than 32 cells are termed blastocysts. These embryos show the development of a blastocoel cavity, typically at the 64 cell stage. The presence of a desired ZNF217 mutation in the cells of the embryo can be detected by methods known to those of skill in the art, e.g., Southern blotting, PCR, DNA sequencing, or other standard methods. Methods for culturing fertilized oocytes to the pre-implantation stage are described, e.g., by Gordon et al., 1984, Methods Enzymol., 101:414; Hogan et al., 1986, MANIPULATION OF THE MOUSE EMBRYO: A LABORATORY MANUAL, C.S.H.L. N.Y. (mouse embryo); Hammer et al., 1985, Nature, 315:680 (rabbit and porcine embryos); Gandolfi et al., 1987, J. Reprod. Fert., 81:23-28; Rexroad et al., 1988, J. Anim. Sci., 66:947-953 (ovine embryos); Eyestone et al., 1989, J. Reprod. Fert., 85:715-720; Camous et al., 1984, J. Reprod. Fert., 72:779-785; and Heyman et al., 1987, Theriogenology, 27:5968 (bovine embryos). Pre-implantation embryos can also be stored frozen for a period pending implantation.

Pre-implantation embryos are transferred to an appropriate female resulting in the birth of a transgenic or chimeric animal, depending upon the stage of development when the transgene is integrated. Chimeric mammals can be bred to form true germline transgenic animals. Chimeric mice and germline transgenic mice can also be ordered from commercial sources (e.g., Deltagen, San Carlos, Calif.).

Other methods for introducing mutations into mammalian cells or animals include recombinase systems, which can be employed to delete all or a portion of a locus of interest. Examples of recombinase systems include, the cre/lox system of bacteriophage P1 (see, e.g., Gu et al., 1994, Science, 265:103-106; Terry et al., 1997, Transgenic Res., 6:349-356) and the FLP/FRT site specific integration system (see, e.g., Dymecki, 1996, Proc. Natl. Acad. Sci. U.S.A., 93:6191-6196). In these systems, sites recognized by the particular recombinase are typically introduced into the genome at a position flanking the portion of the gene that is to be deleted. Introduction of the recombinase into the cells then catalyzes recombination which deletes from the genome the polynucleotide sequence that is flanked by the recombination sites. If desired, one can obtain animals in which only certain cell types lack the ZNF217 gene of interest, e.g., by using a tissue specific promoter to drive the expression of the recombinase. See, e.g., Tsien et al., 1996, Cell 87:1317-26; Brocard et al., 1996, Proc. Natl. Acad. Sci. U.S.A., 93:10887-10890; Wang et al., 1996, Proc. Natl. Acad. Sci. U.S.A., 93:3932-6; and Meyers et al., 1998, Nat. Genet. 18:136-41).

The presence of any mutation in a ZNF217 gene in a cell or animal can be detected using any method described herein, e.g., Southern blot, PCR, DNA sequencing, or using assays based on any ZNF217-dependent cell or organismal property or behavior. See, e.g., Ausubel et al., supra.

IV. Purification of ZNF217 Polypeptides

Either naturally occurring or recombinant ZNF217 polypeptides can be purified for use in functional assays, binding assays, diagnostic assays, and other applications. Naturally occurring ZNF217 polypeptides are purified, e.g., from mammalian tissue such as blood, lymphatic tissue, or any other source of a ZNF217 homolog. Recombinant ZNF217 polypeptides are purified from any suitable bacterial or eukaryotic expression system, e.g., CHO cells or insect cells.

ZNF217 proteins can be purified to substantial purity by standard techniques, including, but not limited to selective precipitation with such substances as ammonium sulfate; column chromatography, immunopurification methods, and others (see, e.g., Scopes, 1993, PROTEIN PURIFICATION: PRINCIPLES AND PRACTICE; U.S. Pat. No. 4,673,641; Ausubel et al., supra; and Sambrook et al., supra).

A number of procedures can be employed when recombinant ZNF217 polypeptide is being purified. For example, proteins having established molecular adhesion properties can be reversibly fused to the ZNF217 polypeptide. With the appropriate ligand, a ZNF217 polypeptide can be selectively adsorbed to a purification column and then freed from the column in a relatively pure form. The fused protein is then removed by enzymatic activity. ZNF217 proteins can also be purified using immunoaffinity columns.

A. Purification of Recombinant ZNF217 Protein

Recombinant proteins are expressed by transformed bacteria or eukaryotic cells such as CHO cells or insect cells in large amounts, typically after promoter induction but expression can be constitutive. Promoter induction with IPTG is one example of an inducible promoter system. Cells are grown according to standard procedures in the art. Fresh or frozen cells are used for isolation of protein.

Proteins expressed in bacteria can form insoluble aggregates (“inclusion bodies”).

Several protocols are suitable for purification of ZNF217 inclusion bodies. For example, purification of inclusion bodies typically involves the extraction, separation and/or purification of inclusion bodies by disruption of bacterial cells, e.g., by incubation in a buffer of 50 mM TRIS/HCL pH 7.5, 50 mM NaCl, 5 mM MgCl₂, 1 mM DTT, 0.1 mM ATP, and 1 mM PMSF. The cell suspension can be lysed using 2-3 passages through a French Press, homogenized using a Polytron (Brinkman Instruments) or sonicated on ice. Alternate methods of lysing bacteria are apparent to those of skill in the art (see, e.g., Sambrook et al., supra; Ausubel et al., supra).

If necessary, the inclusion bodies are solubilized, and the lysed cell suspension is typically centrifuged to remove unwanted insoluble matter. Proteins that formed the inclusion bodies can be renatured by dilution or dialysis with a compatible buffer. Suitable solvents include, but are not limited to, urea (from about 4 M to about 8 M), formamide (at least about 80%, volume/volume basis), and guanidine hydrochloride (from about 4 M to about 8 M). Some solvents which are capable of solubilizing aggregate-forming proteins, for example SDS (sodium dodecyl sulfate) and 70% formic acid, are inappropriate for use in this procedure due to the possibility of irreversible denaturation of the proteins, accompanied by a lack of immunogenicity and/or activity. Although guanidine hydrochloride and similar agents are denaturants, this denaturation is not irreversible and renaturation can occur upon removal (by dialysis, for example) or dilution of the denaturant, allowing re-formation of immunologically and/or biologically active protein. Other suitable buffers are known to those skilled in the art. ZNF217 polypeptides are separated from other bacterial proteins by standard separation techniques, e.g., with Ni-NTA agarose resin.

Alternatively, it is possible to purify ZNF217 polypeptides from bacteria periplasm. After lysis of the bacteria, when a ZNF217 protein is exported into the periplasm of the bacteria, the periplasmic fraction of the bacteria can be isolated by cold osmotic shock in addition to other methods known to skill in the art. To isolate recombinant proteins from the periplasm, the bacterial cells are centrifuged to form a pellet. The pellet is resuspended in a buffer containing 20% sucrose. To lyse the cells, the bacteria are centrifuged and the pellet is resuspended in ice-cold 5 mM MgSO₄ and kept in an ice bath for approximately 10 minutes. The cell suspension is centrifuged and the supernatant decanted and saved. The recombinant proteins present in the supernatant can be separated from the host proteins by standard separation techniques well known to those of skill in the art.

B. Standard Protein Separation Techniques for Purifying ZNF217 Polypeptides

Often as an initial step, particularly if the protein mixture is complex, an initial salt fractionation can separate many of the unwanted host cell proteins (or proteins derived from the cell culture media) from the recombinant protein of interest. The preferred salt is ammonium sulfate. Ammonium sulfate precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol includes adding saturated ammonium sulfate to a protein solution so that the resultant ammonium sulfate concentration is between 20-30%. This concentration will precipitate the most hydrophobic of proteins. The precipitate is then discarded (unless the protein of interest is hydrophobic) and ammonium sulfate is added to the supernatant to a concentration known to precipitate the protein of interest. The precipitate is then solubilized in buffer and the excess salt removed if necessary, either through dialysis or diafiltration. Other methods that rely on solubility of proteins, such as cold ethanol precipitation, are well known to those of skill in the art and can be used to fractionate complex protein mixtures.

The molecular weight of a ZNF217 protein can be used to isolated it from proteins of greater and lesser size using ultrafiltration through membranes of different pore size (for example, Amicon or Millipore membranes). As a first step, the protein mixture is ultrafiltered through a membrane with a pore size that has a lower molecular weight cut-off than the molecular weight of the protein of interest. The retentate of the ultrafiltration is then ultrafiltered against a membrane with a molecular cut off greater than the molecular weight of the protein of interest. The recombinant protein will pass through the membrane into the filtrate. The filtrate can then be chromatographed as described below.

ZNF217 proteins can also be separated from other proteins on the basis of their size, net surface charge, hydrophobicity, and affinity for heterologous molecules. In addition, antibodies raised against proteins can be conjugated to column matrices and the proteins immunopurified. All of these methods are well known in the art. It will be apparent to one of skill that chromatographic techniques can be performed at any scale and using equipment from many different manufacturers (e.g., Pharmacia Biotech).

V. Antibodies to ZNF217

In numerous embodiments of the present invention, antibodies that specifically bind to ZNF217 polypeptides or ZNF217 modulators will be used. Such antibodies have numerous applications, including for the modulation of ZNF217 activity and for immunoassays to detect ZNF217, and variants, derivatives, fragments, and the like, of ZNF217. Immunoassays can be used to qualitatively or quantitatively analyze the ZNF217 polypeptide. A general overview of the applicable technology can be found in Harlow & Lane, 1988, ANTIBODIES: A LABORATORY MANUAL.

A. Production of Nonhuman Antibodies

Methods of producing polyclonal and monoclonal antibodies that react specifically with ZNF217 polypeptides or ZNF217 molulators are known to those of skill in the art (see, e.g., Coligan, 1991, CURRENT PROTOCOLS IN IMMUNOLOGY; Harlow & Lane, supra; Goding, MONOCLONAL ANTIBODIES: PRINCIPLES AND PRACTICE (2^{nd.} Ed. 1986); and Kohler & Milstein, 1975, Nature 256:495-497. Such techniques include antibody preparation by selection of antibodies from libraries of recombinant antibodies in phage or similar vectors, as well as preparation of polyclonal and monoclonal antibodies by immunizing rabbits or mice (see, e.g., Huse et al., 1989, Science 246:1275-1281; Ward et al., 1989, Nature 341:544-546).

A number of ZNF217-comprising immunogens can be used to produce antibodies specifically reactive with a ZNF217 polypeptide. For example, a recombinant ZNF217 protein, or an antigenic fragment thereof, is isolated as described herein. Recombinant protein can be expressed in eukaryotic or prokaryotic cells as described above, and purified as generally described above. Recombinant protein is the preferred immunogen for the production of monoclonal or polyclonal antibodies. Alternatively, a synthetic peptide derived from the sequences disclosed herein and conjugated to a carrier protein can be used an immunogen. Naturally occurring protein can also be used either in pure or impure form. The product is then injected into an animal capable of producing antibodies. Either monoclonal or polyclonal antibodies can be generated, for subsequent use in immunoassays to measure the protein.

Methods of production of polyclonal antibodies are known to those of skill in the art. An inbred strain of mice (e.g., BALB/C mice) or rabbits is immunized with the protein using a standard adjuvant, such as Freund's adjuvant, and a standard immunization protocol. The animal's immune response to the immunogen preparation is monitored by taking test bleeds and determining the titer of reactivity to the ZNF217 polypeptide. When appropriately high titers of antibody to the immunogen are obtained, blood is collected from the animal and antisera are prepared. Further fractionation of the antisera to enrich for antibodies reactive to the protein can be done if desired (see Harlow & Lane, supra).

Monoclonal antibodies can be obtained by various techniques familiar to those skilled in the art. Briefly, spleen cells from an animal immunized with a desired antigen are immortalized, commonly by fusion with a myeloma cell (see Kohler & Milstein, 1976, Eur. J. Immunol. 6:511-519). Alternative methods of immortalization include transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells can be enhanced by various techniques, including injection into the peritoneal cavity of a vertebrate host. Alternatively, one can isolate DNA sequences which encode a monoclonal antibody or a binding fragment thereof by screening a DNA library from human B cells according to the general protocol outlined by Huse et al., 1989, Science 246:1275-1281.

Monoclonal antibodies and polyclonal sera are collected and titered against the immunogen protein in an immunoassay, for example, a solid phase immunoassay with the immunogen immobilized on a solid support. Typically, polyclonal antisera with a titer of 10⁴ or greater are selected and tested for their cross reactivity against non-ZNF217 proteins, or even related proteins from other organisms, using a competitive binding immunoassay. Specific polyclonal antisera and monoclonal antibodies will usually bind with a K_{d} of at least about 0.1 mM, more usually at least about 1 μM, optionally at least about 0.1 μM or better, and optionally 0.01 μM or better.

B. Chimeric and Humanized Antibodies

Chimeric and humanized antibodies have the same or similar binding specificity and affinity as a mouse or other nonhuman antibody that provides the starting material for construction of a chimeric or humanized antibody. Some chimeric or humanized antibodies have affinities within a factor of 2-fold, 5-fold or 10-fold that of a mouse. Chimeric antibodies are antibodies whose light and heavy chain genes have been constructed, typically by genetic engineering, from immunoglobulin gene segments belonging to different species. For example, the variable (V) segments of the genes from a mouse monoclonal antibody can be joined to human constant (C) segments, such as IgG₁, IgG₂, IgG₃ and IgG₄. A typical chimeric antibody is thus a hybrid protein consisting of the V or antigen-binding domain from a mouse antibody and the C or effector domain from a human antibody.

Humanized antibodies have variable region framework residues substantially from a human antibody (termed an acceptor antibody) and complementarity determining regions substantially from a nonhuman antibody such as a mouse-antibody, (referred to as the donor immunoglobulin). See Queen et al., 1989, Proc. Natl. Acad. Sci. USA 86:10029-33 and WO 90/07861, U.S. Pat. Nos. 5,693,762, 5,693,761, 5,585,089, 5,530,101 and Winter, U.S. Pat. No. 5,225,539 (each of which is herein incorporated by reference in its entirety for all purposes). The constant region(s), if present, are also substantially or entirely from a human immunoglobulin. The human variable domains are usually chosen from human antibodies whose framework sequences exhibit a high degree of sequence identity with the murine variable region domains from which the CDRs were derived. The heavy and light chain variable region framework residues can be derived from the same or different human antibody sequences. The human antibody sequences can be the sequences of naturally occurring human antibodies or can be consensus sequences of several human antibodies. See Carter et al., WO 92/22653. Certain amino acids from the human variable region framework residues are selected for substitution based on their possible influence on CDR conformation and/or binding to antigen. Investigation of such possible influences is by modeling, examination of the characteristics of the amino acids at particular locations, or empirical observation of the effects of substitution or mutagenesis of particular amino acids.

For example, when an amino acid differs between a murine variable region framework residue and a selected human variable region framework residue, the human framework amino acid should usually be substituted by the equivalent framework amino acid from the mouse antibody when it is reasonably expected that the amino acid: (1) noncovalently binds antigen directly, (2) is adjacent to a CDR region, (3) otherwise interacts with a CDR region (e.g. is within about 6A of a CDR region), or (4) participates in the _{VL}-_{VH} interface.

Other candidates for substitution are acceptor human framework amino acids that are unusual for a human immunoglobulin at that position. These amino acids can be substituted with amino acids from the equivalent position of the donor antibody or from the equivalent positions of more typical human immunoglobulins. Other candidates for substitution are acceptor human framework amino acids that are unusual for a human immunoglobulin at that position. The variable region frameworks of humanized immunoglobulins usually show at least 85% sequence identity to a human variable region framework sequence or consensus of such sequences.

C. Human Antibodies

Human antibodies against ZNF217 or ZNF217 modulators can be generated by a variety of techniques described below. Some human antibodies are selected by competitive binding experiments, or otherwise, to have the same epitope specificity as a particular mouse antibody, such as one of the mouse monoclonals described in the Examples. Human antibodies can also be screened for a particular epitope specificity by using only a fragment of ZNF217 or a ZNF217 modulator as the immunogen, and/or by screening antibodies against a collection of deletion mutants of ZNF217 or a ZNF217 modulator.

1. Trioma Methodology

The basic approach and an exemplary cell fusion partner, SPAZ-4, for use in this approach have been described by Oestberg et al., 1983, Hybridoma 2:361-67; Oestberg, U.S. Pat. No.4,634,664; and Engleman et al., U.S. Pat. No. 4,634,666 (each of which is incorporated by reference in their entirety for all purposes). The antibody-producing cell lines obtained by this method are called triomas, because they are descended from three cells—two human and one mouse. Initially, a mouse myeloma line is fused with a human B-lymphocyte to obtain a non-antibody-producing xenogeneic hybrid cell, such as the SPAZ-4 cell line described by Oestberg, supra. The xenogeneic cell is then fused with an immunized human B-lymphocyte to obtain an antibody-producing trioma cell line. Triomas have been found to produce antibody more stably than ordinary hybridomas made from human cells.

The immunized B-lymphocytes are obtained from the blood, spleen, lymph nodes or bone marrow of a human donor. If antibodies against a specific antigen or epitope are desired, it is preferable to use that antigen or epitope thereof for immunization. Immunization can be either in vivo or in vitro. For in vivo immunization, B cells are typically isolated from a human immunized with A, a fragment thereof, larger polypeptide containing A or fragment, or an anti-idiotypic antibody to an antibody to A. In some methods, B cells are isolated from the same patient who is ultimately to be administered antibody therapy. For in vitro immunization, B-lymphocytes are typically exposed to antigen for a period of 7-14 days in a media such as RPMI-1640 (see Engleman, supra) supplemented with 10% human plasma.

The immunized B-lymphocytes are fused to a xenogeneic hybrid cell such as SPAZ-4 by well known methods. For example, the cells are treated with 40-50% polyethylene glycol of MW 1000-4000, at about 37° C., for about 5-10 min. Cells are separated from the fusion mixture and propagated in media selective for the desired hybrids (e.g., HAT or AH). Clones secreting antibodies having the required binding specificity are identified by assaying the trioma culture medium for the ability to bind to A or a fragment thereof. Triomas producing human antibodies having the desired specificity are subcloned by the limiting dilution technique and grown in vitro in culture medium. The trioma cell lines obtained are then tested for the ability to bind A or a fragment thereof.

Although triomas are genetically stable they do not produce antibodies at very high levels. Expression levels can be increased by cloning antibody genes from the trioma into one or more expression vectors, and transforming the vector into standard mammalian, bacterial or yeast cell lines.

2. Transgenic Non-Human Mammals

Human antibodies against ZNF217 can also be produced from non-human transgenic mammals having transgenes encoding at least a segment of the human immunoglobulin locus as discussed supra. Usually, the endogenous immunoglobulin locus of such transgenic mammals is functionally inactivated. Preferably, the segment of the human immunoglobulin locus includes unrearranged sequences of heavy and light chain components. Both inactivation of endogenous immunoglobulin genes and introduction of exogenous immunoglobulin genes can be achieved by targeted homologous recombination, or by introduction of YAC chromosomes. The transgenic mammals resulting from this process are capable of functionally rearranging the immunoglobulin component sequences, and expressing a repertoire of antibodies of various isotypes encoded by human immunoglobulin genes, without expressing endogenous immunoglobulin genes. The production and properties of mammals having these properties are described in detail by, e.g., Lonberg et al., WO93/12227 (1993); U.S. Pat. Nos. 5,877,397, 5,874,299, 5,814,318, 5,789,650, 5,770,429, 5,661,016, 5,633,425, 5,625,126, 5,569,825, 5,545,806, Nature 148:1547-53 (1994), Fishwild et al., 1996, Nature Biotechnology 14, 845-51, Kucherlapati, WO 91/10741 (1991) (each of which is incorporated by reference in its entirety for all purposes). Transgenic mice are particularly suitable. Anti-ZNF217 antibodies are obtained by immunizing a transgenic nonhuman mammal, such as described by Lonberg or Kucherlapati, supra, with a ZNF217 or subunit or a fragment thereof. Monoclonal antibodies are prepared by, e.g., fusing B-cells from such mammals to suitable myeloma cell lines using conventional Kohler-Milstein technology. Human polyclonal antibodies can also be provided in the form of serum from humans immunized with an immunogenic agent. Optionally, such polyclonal antibodies can be concentrated by affinity purification using ZNF217 or a ZNF217 modulator as an affinity reagent.

3. Phage Display Methods

A further approach for obtaining human anti-ZNF217 or an anti-ZNF217 modulator to screen a DNA library from human B cells according to the general protocol outlined by Huse et al., 1989, Science 246:1275-81. As described for trioma methodology, such B cells can be obtained from a human immunized with ZNF217 or an anti-ZNF217 modulator or fragments thereof. Optionally, such B cells are obtained from a patient who is ultimately to receive antibody treatment. Antibodies binding to an antigen of interest or a fragment thereof are selected. Sequences encoding such antibodies (or a binding fragments) are then cloned and amplified. The protocol described by Huse is rendered more efficient in combination with phage-display technology. See, e.g., Dower et al., WO 91/17271 and McCafferty et al. WO 92/01047, U.S. Pat. Nos. 5,877,218, 5,871,907, 5,858,657, 5,837,242, 5,733,743 and 5,565,332, 5,969,108, 6,172,197 (each of which is incorporated by reference in its entirety for all purposes). Additional methods for selecting and labeling antibodies, or other proteins, that bind to a particular ligand are described by U.S. Pat. Nos. 5,994,519 and 6,180,336.

In phage display methods, libraries of phage are produced in which members display different antibodies on their outer surfaces. Antibodies are usually displayed as Fv or Fab fragments. Phage displaying antibodies with a desired specificity are selected by affinity enrichment to ZNF217 or an anti-ZNF217 modulator subunit or fragment thereof.

In a variation of the phage-display method, human antibodies having the binding specificity of a selected murine antibody can be produced. See Winter, WO 92/20791. In this method, either the heavy or light chain variable region of the selected murine antibody is used as a starting material. If, for example, a light chain variable region is selected as the starting material, a phage library is constructed in which members display the same light chain variable region (i.e., the murine starting material) and a different heavy chain variable region. The heavy chain variable regions are obtained from a library of rearranged human heavy chain variable regions. A phage showing strong specific binding for A (e.g., at least 10⁸ and preferably at least 10⁹ M⁻¹) is selected. The human heavy chain variable region from this phage then serves as a starting material for constructing a further phage library. In this library, each phage displays the same heavy chain variable region (i.e., the region identified from the first display library) and a different light chain variable region. The light chain variable regions are obtained from a library of rearranged human variable light chain regions. Again, phage showing strong specific binding for a desired target are selected. These phage display the variable regions of completely human anti-ZNF217 or anti-ZNF217 modulator antibodies. These antibodies usually have the same or similar epitope specificity as the murine starting material.

4. Selection of Constant Region

The heavy and light chain variable regions of chimeric, humanized, or human antibodies can be linked to at least a portion of a human constant region. The choice of constant region depends, in part, whether antibody-dependent complement and/or cellular mediated toxicity is desired. For example, isotopes IgG₁ and IgG₃ have complement activity and isotypes IgG₂ and IgG₄ do not. Choice of isotype can also affect passage of antibody into the brain. Light chain constant regions can be lambda or kappa. Antibodies can be expressed as tetramers containing two light and two heavy chains, as separate heavy chains, light chains, as Fab, Fab′ F(ab′)2, and Fv, or as single chain antibodies in which heavy and light chain variable domains are linked through a spacer.

5. Expression of Recombinant Antibodies

Chimeric, humanized and human antibodies are typically produced by recombinant expression. Recombinant polynucleotide constructs typically include an expression control sequence operably linked to the coding sequences of antibody chains, including naturally-associated or heterologous promoter regions. Preferably, the expression control sequences are eukaryotic promoter systems in vectors capable of transforming or transfecting eukaryotic host cells. Once the vector has been incorporated into the appropriate host, the host is maintained under conditions suitable for high level expression of the nucleotide sequences, and the collection and purification of the crossreacting antibodies.

These expression vectors are typically replicable in the host organisms either as episomes or as an integral part of the host chromosomal DNA. Commonly, expression vectors contain selection markers, e.g., ampicillin-resistance or hygromycin-resistance, to permit detection of those cells transformed with the desired DNA sequences.

E. coli is one prokaryotic host particularly useful for cloning the DNA sequences of the present invention. Microbes, such as yeast are also useful for expression. Saccharomyces is a preferred yeast host, with suitable vectors having expression control sequences, an origin of replication, termination sequences and the like as desired. Typical promoters include 3-phosphoglycerate kinase and other glycolytic enzymes. Inducible yeast promoters include, among others, promoters from alcohol dehydrogenase, isocytochrome C, and enzymes responsible for maltose and galactose utilization.

Mammalian cells are a preferred host for expressing nucleotide segments encoding immunoglobulins or fragments thereof. See Winnacker, FROM GENES TO CLONES, (VCH Publishers, N.Y., 1987). A number of suitable host cell lines capable of secreting intact heterologous proteins have been developed in the art, and include CHO cell lines, various COS cell lines, HeLa cells, L cells and myeloma cell lines. Preferably, the cells are nonhuman. Expression vectors for these cells can include expression control sequences, such as an origin of replication, a promoter, an enhancer (Queen et al., 1986, Immunol. Rev. 89:49-68), and necessary processing information sites, such as ribosome binding sites, RNA splice sites, polyadenylation sites, and transcriptional terminator sequences. Preferred expression control sequences are promoters derived from endogenous genes, cytomegalovirus, SV40, adenovirus, bovine papillomavirus, and the like. See Co et al., 1992, J. Immunol. 148:1149-54.

Alternatively, antibody coding sequences can be incorporated in transgenes for introduction into the genome of a transgenic animal and subsequent expression in the milk of the transgenic animal (see, e.g., U.S. Pat. Nos. 5,741,957, 5,304,489, 5,849,992). Suitable transgenes include coding sequences for light and/or heavy chains in operable linkage with a promoter and enhancer from a mammary gland specific gene, such as casein or beta lactoglobulin.

The vectors containing the DNA segments of interest can be transferred into the host cell by well-known methods, depending on the type of cellular host. For example, calcium chloride transfection is commonly utilized for prokaryotic cells, whereas calcium phosphate treatment, electroporation, lipofection, biolistics or viral-based transfection can be used for other cellular hosts. Other methods used to transform mammalian cells include the use of polybrene, protoplast fusion, liposomes, electroporation, and microinjection (see generally, Sambrook et al., supra). For production of transgenic animals, transgenes can be microinjected into fertilized oocytes, or can be incorporated into the genome of embryonic stem cells, and the nuclei of such cells transferred into enucleated oocytes.

Once expressed, antibodies can be purified according to standard procedures of the art, including HPLC purification, column chromatography, gel electrophoresis and the like (see generally, Scopes, 1982, PROTEIN PURIFICATION (Springer-Verlag, N.Y.).

D. Immunological Binding Assays

Once ZNF217-specific antibodies are available, individual ZNF217 proteins can be detected by a variety of immunoassay methods. For a review of the general immunoassays, see also METHODS IN CELL BIOLOGY: ANTIBODIES IN CELL BIOLOGY, Vol. 37 (Asai, Ed. 1993); BASIC AND CLINICAL IMMUNOLOGY (Stites & Terr, eds., 7^{th.} Ed. 1991). Moreover, the immunoassays of the present invention can be performed in any of several configurations, which are reviewed extensively in ENZYME IMMUNOASSAY (Maggio, Ed., 1980); and Harlow & Lane, supra. Immunological binding assays (or immunoassays) typically use an antibody that specifically binds to a protein or antigen of choice (in this case a ZNF217 protein or an antigenic subsequence thereof). The antibody (e.g., anti-ZNF217) can be produced by any of a number of means well known to those of skill in the art and as described above. Immunoassays also often use a labeling agent to specifically bind to and label the complex formed by the antibody and antigen. The labeling agent can itself be one of the moieties comprising the antibody/antigen complex. Thus, the labeling agent can be a labeled ZNF217 polypeptide or a labeled anti-ZNF217 antibody. Alternatively, the labeling agent can be a third moiety, such a secondary antibody, that specifically binds to the antibody/ZNF217 complex (a secondary antibody is typically specific to antibodies of the species from which the first antibody is derived). Other proteins capable of specifically binding immunoglobulin constant regions, such as protein A or protein G, can also be used as the label agent. These proteins exhibit a strong nonimmunogenic reactivity with immunoglobulin constant regions from a variety of species (see, e.g., Kronval et al., 1973, J. Immunol. 111:1401-1406; Akerstrom et al., 1985, J. Immunol. 135:2589-2542). The labeling agent can be modified with a detectable moiety, such as biotin, to which another molecule can specifically bind, such as streptavidin. A variety of detectable moieties are well known to those skilled in the art.

Throughout the assays, incubation and/or washing steps can be required after each combination of reagents. Incubation steps can vary from about 5 seconds to several hours, optionally from about 5 minutes to about 24 hours. However, the incubation time will depend upon the assay format, antigen, volume of solution, concentrations, and the like. Usually, the assays will be carried out at ambient temperature, although they can be conducted over a range of temperatures, such as 10° C. to 40° C.

1. Noncompetitive Assay Formats

Immunoassays for detecting a ZNF217 protein in a sample can be either competitive or noncompetitive. Noncompetitive immunoassays are assays in which the amount of antigen is directly measured. In one preferred “sandwich” assay, for example, the anti-ZNF217 antibodies can be bound directly to a solid substrate on which they are immobilized. These immobilized antibodies then capture the ZNF217 protein present in the test sample. The ZNF217 protein thus immobilized is then bound by a labeling agent, such as a second ZNF217 antibody bearing a label. Alternatively, the second antibody can lack a label, but it can, in turn, be bound by a labeled third antibody specific to antibodies of the species from which the second antibody is derived. The second or third antibody is typically modified with a detectable moiety, such as biotin, to which another molecule specifically binds, e.g., streptavidin, to provide a detectable moiety.

2. Competitive Assay Formats

In competitive assays, the amount of ZNF217 protein present in the sample is measured indirectly by measuring the amount of a known, added (exogenous) ZNF217 protein displaced (competed away) from an anti-ZNF217 antibody by the unknown ZNF217 protein present in a sample. In one competitive assay, a known amount of ZNF217 protein is added to a sample and the sample is then contacted with an antibody that specifically binds to the ZNF217 protein. The amount of exogenous ZNF217 protein bound to the antibody is inversely proportional to the concentration of ZNF217 protein present in the sample. In a particularly preferred embodiment, the antibody is immobilized on a solid substrate. The amount of ZNF217 protein bound to the antibody can be determined either by measuring the amount of ZNF217 protein present in a ZNF217/antibody complex, or alternatively by measuring the amount of remaining uncomplexed protein. The amount of ZNF217 protein can be detected by providing a labeled ZNF217 molecule.

A hapten inhibition assay is another preferred competitive assay. In this assay, the known ZNF217 protein is immobilized on a solid substrate. A known amount of anti-ZNF217 antibody is added to the sample, and the sample is then contacted with the immobilized ZNF217. The amount of anti-ZNF217 antibody bound to the known immobilized ZNF217 protein is inversely proportional to the amount of ZNF217 protein present in the sample. Again, the amount of immobilized antibody can be detected by detecting either the immobilized fraction of antibody or the fraction of the antibody that remains in solution. Detection can be direct where the antibody is labeled or indirect by the subsequent addition of a labeled moiety that specifically binds to the antibody as described above.

3. Cross-reactivity Determinations

Immunoassays in the competitive binding format can also be used for crossreactivity determinations. For example, a protein at least partially encoded by the naturally occurring ZNF217 can be immobilized to a solid support. Proteins (e.g., ZNF217 proteins and homologs) are added to the assay that compete for binding of the antisera to the immobilized antigen. The ability of the added proteins to compete for binding of the antisera to the immobilized protein is compared to the ability of the naturally occurring ZNF217 polypeptide to compete with itself. The percent cross-reactivity for the above proteins is calculated, using standard calculations. Those antisera with less than 10% cross-reactivity with each of the added proteins listed above are selected and pooled. The cross-reacting antibodies are optionally removed from the pooled antisera by immunoabsorption with the added considered proteins, e.g., distantly related homologs.

The immunoabsorbed and pooled antisera are then used in a competitive binding immunoassay as described above to compare a second protein, thought to be perhaps an allele or polymorphic variant of a ZNF217 protein, to the immunogen protein (i.e., the naturally occurring ZNF217 protein). In order to make this comparison, the two proteins are each assayed at a wide range of concentrations and the amount of each protein required to inhibit 50% of the binding of the antisera to the immobilized protein is determined. If the amount of the second protein required to inhibit 50% of binding is less than 10 times the amount of the naturally occurring protein that is required to inhibit 50% of binding, then the second protein is said to specifically bind to the polyclonal antibodies generated to a ZNF217 immunogen.

Polyclonal antibodies that specifically bind to a ZNF217 protein from a particular species can be make by subtracting out cross-reactive antibodies using ZNF217 homologs. For example, antibodies specific to human ZNF217 can be made by subtracting out antibodies that are cross-reactive with mouse ZNF217. In an analogous fashion, antibodies specific to a particular ZNF217 protein can be made in an organism with multiple ZNF217 genes.

4. Other Assay Formats

Western blot (immunoblot) analysis is used to detect and quantify the presence of ZNF217 protein in a sample. The technique generally comprises separating sample proteins by gel electrophoresis on the basis of molecular weight, transferring the separated proteins to a suitable solid support, (such as a nitrocellulose filter, a nylon filter, or derivatized nylon filter), and incubating the sample with the antibodies that specifically bind the ZNF217 protein. The anti-ZNF217 polypeptide antibodies specifically bind to the ZNF217 polypeptide on the solid support. These antibodies can be directly labeled or alternatively can be subsequently detected using labeled antibodies (e.g., labeled sheep anti-mouse antibodies) that specifically bind to the anti-ZNF217 antibodies.

Additional assay include immunocytochemical assays that identify the presence of ZNF217 in particular cells and the subcellular localization of ZNF217. Such assay are performed using standard techniques (see, e.g., CURRENT PROTOCOLS IN IMMUNOLOGY, 1991) Other assay formats include liposome immunoassays (LIA), which use liposomes designed to bind specific molecules (e.g., antibodies) and release encapsulated reagents or markers. The released chemicals are then detected according to standard techniques (see Monroe et al., 1986, Amer. Clin. Prod. Rev. 5:34-41).

5. Reduction of nonspecific Binding

One of skill in the art will appreciate that it is often desirable to minimize nonspecific binding in immunoassays. Particularly, where the assay involves an antigen or antibody immobilized on a solid substrate it is desirable to minimize the amount of nonspecific binding to the substrate. Means of reducing such nonspecific binding are well known to those of skill in the art. Typically, this technique involves coating the substrate with a proteinaceous composition. In particular, protein compositions such as bovine serum albumin (BSA), nonfat powdered milk, and gelatin are widely used with powdered milk being most preferred.

6. Labels

The particular label or detectable group used in the assay is not a critical aspect of the invention, as long as it does not significantly interfere with the specific binding of the antibody used in the assay. The detectable group can be any material having a detectable physical or chemical property. Such detectable labels have been well-developed in the field of immunoassays and, in general, most any label useful in such methods can be applied to the present invention. Thus, a label is any composition detectable by spectroscopic, photochemical, biochemical, immunochemical, electrical, optical or chemical means. Useful labels in the present invention include magnetic beads (e.g., DYNABEADS™), fluorescent dyes (e.g., fluorescein isothiocyanate, Texas red, rhodamine, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold or colored glass or plastic beads (e.g., polystyrene, polypropylene, latex, etc.).

The label can be coupled directly or indirectly to the desired component of the assay according to methods well known in the art. As indicated above, a wide variety of labels can be used, with the choice of label depending on sensitivity required, ease of conjugation with the compound, stability requirements, available instrumentation, and disposal provisions.

Nonradioactive labels are often attached by indirect means. Generally, a ligand molecule (e.g., biotin) is covalently bound to the molecule. The ligand then binds to another molecule (e.g., streptavidin), which is either inherently detectable or covalently bound to a signal system, such as a detectable enzyme, a fluorescent compound, or a chemiluminescent compound. The ligands and their targets can be used in any suitable combination with antibodies that recognize a ZNF217 protein, or secondary antibodies that recognize anti-ZNF217.

The molecules can also be conjugated directly to signal generating compounds, e.g., by conjugation with an enzyme or fluorophore. Enzymes of interest as labels will primarily be hydrolases, particularly phosphatases, esterases and glycosidases, or oxidases, particularly peroxidases. Fluorescent compounds include fluorescein and its derivatives, rhodamine and its derivatives, dansyl, umbelliferone, and the like. Chemiluminescent compounds include luciferin, and 2,3-dihydrophthalazinediones, e.g., luminol. For a review of various labeling or signal producing systems that can be used, see, U.S. Pat. No. 4,391,904.

Means of detecting labels are well known to those of skill in the art. Thus, for example, where the label is a radioactive label, means for detection include a scintillation counter or photographic film as in autoradiography. Where the label is a fluorescent label, it can be detected by exciting the fluorochrome with the appropriate wavelength of light and detecting the resulting fluorescence. The fluorescence can be detected visually, by means of photographic film, by the use of electronic detectors such as charge coupled devices (CCDs) or photomultipliers and the like. Similarly, enzymatic labels can be detected by providing the appropriate substrates for the enzyme and detecting the resulting reaction product. Finally simple colorimetric labels can be detected simply by observing the color associated with the label. Thus, in various dipstick assays, conjugated gold often appears pink, while various conjugated beads appear the color of the bead.

Some assay formats do not require the use of labeled components. For instance, agglutination assays can be used to detect the presence of the target antibodies. In this case, antigen-coated particles are agglutinated by samples comprising the target antibodies. In this format, none of the components need be labeled and the presence of the target antibody is detected by simple visual inspection.

VI. Modulating ZFN217 Activity

A. Assays for Modulators of ZNF217 Proteins

In numerous embodiments of this invention, the level of ZNF217 activity will be modulated in a cell by administering to the cell, in vivo or in vitro, any of a large number of ZNF217-modulating molecules, e.g., polypeptides, antibodies, amino acids, nucleotides, lipids, carbohydrates, or any organic or inorganic molecule.

To identify molecules capable of modulating ZNF217, assays will be performed to detect the effect of various compounds on ZNF217 activity in a cell. The activity of ZNF217 polypeptides can be assessed using a variety of in vitro and in vivo assays to determine functional, chemical, and physical effects, e.g., measuring the binding of ZNF217 to other molecules (e.g., radioactive binding), measuring ZNF217 protein and/or RNA levels, or measuring other aspects of ZNF217 polypeptides, e.g., phosphorylation levels, transcription levels, receptor activity, ligand binding and the like. Such assays can be used to test for both activators and inhibitors of ZNF217 proteins. Modulators thus identified are useful for, e.g., many diagnostic and therapeutic applications.

The ZNF217 protein of the assay will typically be a recombinant or naturally occurring polypeptide or a conservatively modified variant thereof. Alternatively, the ZNF217 protein of the assay will be derived from a eukaryote and include an amino acid subsequence having amino acid sequence identity to the naturally occurring ZNF217 protein. Generally, the amino acid sequence identity will be at least 70%, optionally at least 75%, 85%, or 86%, 87%, 88%, 89%, 90 %, 91%, 92%, 93%, 94%, 95%, 96%, 97%, 98% or greater. Optionally, the polypeptide of the assays will comprise a domain of a ZNF217 protein. In certain embodiments, a domain of a ZNF217 protein, e.g., a zinc finger binding domain, is bound to a solid substrate and used, e.g., to isolate any molecules that can bind to and/or modulate their activity. In certain embodiments, a domain of a ZNF217 polypeptide, e.g., an N-terminal domain, a C-terminal domain, is fused to a heterologous polypeptide, thereby forming a chimeric polypeptide. Such chimeric polypeptides are also useful, e.g., in assays to identify modulators of ZNF217.

Samples or assays that are treated with a potential ZNF217 protein inhibitor or activator are compared to control samples without the test compound, to examine the extent of modulation. Control samples (untreated with activators or inhibitors) are assigned a relative ZNF217 activity value of 100. Inhibition of a ZNF217 protein is achieved when the ZNF217 activity value relative to the control is about 90%, optionally about 50%, optionally about 25-0%. Activation of a ZNF217 protein is achieved when the ZNF217 activity value relative to the control is about 110%, optionally about 150%, 200-500%, or about 1000-2000%.

The effects of the test compounds upon the function of the polypeptides can be measured by examining any of the parameters described above. Any suitable physiological change that affects ZNF217 activity can be used to assess the influence of a test compound on the polypeptides of this invention. When the functional consequences are determined using intact cells or animals, one can also measure a variety of effects such as changes in cell growth or changes in cell-cell interactions.

Modulators of ZNF217 that act by modulating ZNF217 gene expression can also be identified. For example, a host cell containing a ZNF217 protein of interest is contacted with a test compound for a sufficient time to effect any interactions, and then the level of gene expression is measured. The amount of time to effect such interactions can be empirically determined, such as by running a time course and measuring the level of transcription as a function of time. The amount of transcription can be measured using any method known to those of skill in the art to be suitable. For example, mRNA expression of the protein of interest can be detected using Northern blots or by detecting their polypeptide products using immunoassays.

B. Assays for ZNF217-Interacting Compounds

In certain embodiments, assays will be performed to identify molecules that physically interact with ZNF217 proteins. Such molecules can be any type of molecule, including polypeptides, polynucleotides, amino acids, nucleotides, carbohydrates, lipids, or any other organic or inorganic molecule. Such molecules can represent molecules that normally interact with ZNF217 or can be synthetic or other molecules that are capable of interacting with ZNF217 and that can potentially be used as lead compounds to identify classes of molecules that can interact with and/or modulate ZNF217. Such assays can represent physical binding assays, such as affinity chromatography, immunoprecipitation, two-hybrid screens, or other binding assays, or can represent genetic assays.

In any of the binding or functional assays described herein, in vivo or in vitro, any ZNF217 protein, or any derivative, variation, homolog, or fragment of a naturally occurring ZNF217 protein, can be used. Preferably, the ZNF217 protein has at least about 85% identity to the amino acid sequence of the naturally occurring ZNF217 protein. In numerous embodiments, a fragment of a ZNF217 protein is used. Such fragments can be used alone, in combination with other ZNF217 fragments, or in combination with sequences from heterologous proteins, e.g., the fragments can be fused to a heterologous polypeptides, thereby forming a chimeric polypeptide.

Compounds that interact with ZNF217 proteins can be isolated based on an ability to specifically bind to a ZNF217 protein or fragment thereof. In numerous embodiments, the ZNF217 protein or protein fragment will be attached to a solid support. In one embodiment, affinity columns are made using the ZNF217 polypeptide, and physically-interacting molecules are identified. It will be apparent to one of skill that chromatographic techniques can be performed at any scale and using equipment from many different manufactures (e.g., Pharmacia Biotechnology). In addition, molecules that interact with ZNF217 proteins in vivo can be identified by co-immunoprecipitation or other methods, i.e., immunoprecipitating ZNF217 protein using anti-ZNF217 antibodies from a cell or cell extract, and identifying compounds, e.g., proteins, that are precipitated along with the ZNF217 protein. Such methods are well known to those of skill in the art and are taught, e.g., in Ausubel et al., Sambrook et al., and Harlow & Lane, all supra.

C. Reducing ZNF217 Activity Levels In Cells

In preferred embodiments, this invention provides methods of treating a cancer by reducing ZNF217 levels in a cell. Typically, such methods are used to reduce an elevated level of ZNF217, e.g., an elevated level in a cancerous cell, and can be performed in any of a number of ways, e.g., lowering the copy number of ZNF217 genes or decreasing the level of ZNF217 mRNA, protein, or protein activity in a cell. Preferably, the level of ZNF217 activity is lowered to a level typical of a normal, cancer-free cell, but the level can be reduced to any level that is sufficient to decrease the proliferation or steroid production of the cell, including to levels above or below those typical of normal cells. Preferably, such methods involve the use of inhibitors of ZNF217, where an “inhibitor of ZNF217” is a molecule that acts to reduce ZNF217 polynucleotide levels, ZNF217 polypeptide levels and/or ZNF217 protein activity. Such inhibitor s include, but are not limited to, antisense polynucleotides, siRNA, ribozymes, antibodies, dominant negative ZNF217 forms, and small molecule inhibitors of ZNF217.

In preferred embodiments, ZNF217 levels will be reduced so as to reduce the growth or proliferation of a cancer cell with elevated ZNF217 levels. The proliferation of a cell refers to the rate at which the cell or population of cells divides, or to the extent to which the cell or population of cells divides or increases in number. Proliferation can reflect any of a number of factors, including the rate of cell growth and division and the rate of cell death. Without being bound by the following offered theory, it is suggested that the amplification and/or overexpression of the ZNF217 gene in cancer cells, e.g., breast cancer cells, suppresses programmed cell death induced by both telomere dysfunction and the common chemotherapeutic agent doxorubicin. The prosurvival activity of ZNF217 can act throughout carcinogenesis by promoting immortalization and protecting against the destruction of malignant cells by agents inducing double strand breaks (DSBs), thus enabling tumor progression and conferring a poor prognosis on breast cancer patients. Thus, by decreasing the ZNF217 activity in a cell, e.g., a breast cancer cell, ZNF217 inhibitors can be important for breast cancer prevention and for enhancing the efficacy of common therapeutic agents such as doxorubicin. The efficacy of doxorubicin derives from its ability to inhibit topoisomerase II resulting in DSBs and ATM/p53-mediated apoptosis (see Chabner, B and Longo, D., eds., 1996, (Philedelphia and New York, Lippincott-Raven) CANCER CHEMOTHERAPY AND BIOTHERAPY PRINCIPLES AND PRACTICE, 2^{nd.} Ed.; this reference is herein incorporated by reference in its entirety for all purposes).

The ability of any of the present compounds to affect ZNF217 activity can be determined based on any of a number of factors, including, but not limited to, a level of ZNF217 polynucleotide, e.g., mRNA or gDNA, the level of ZNF217 polypeptide, the degree of binding of a compound to a ZNF217 polynucleotide or polypeptide, ZNF217 intracellular localization, or any functional properties of ZNF217 protein, such as the ability of ZNF217 activity to effect cholesterol translocation into the mitochondria and the resulting steroid hormone synthesis.

1. Inhibitors of ZNF217 Polynucleotides

a) Antisense Polynucleotides

In certain embodiments, ZNF217 activity is downregulated, or entirely inhibited, by the use of antisense polynucleotide, i.e., a nucleic acid complementary to, and which can preferably hybridize specifically to, a coding mRNA nucleic acid sequence, e.g, ZNF217 mRNA, or a subsequence thereof. Binding of the antisense polynucleotide to the ZNF217 mRNA reduces the translation and/or stability of the ZNF217 mRNA.

In the context of this invention, antisense polynucleotides can comprise naturally-occurring nucleotides, or synthetic species formed from naturally-occurring subunits or their close homologs. Antisense polynucleotides can also have altered sugar moieties or inter-sugar linkages. Exemplary among these are the phosphorothioate and other sulfur containing species which are known for use in the art. All such analogs are comprehended by this invention so long as they function effectively to hybridize with ZNF217 mRNA.

Such antisense polynucleotides can readily be synthesized using recombinant means, or can be synthesized in vitro. Equipment for such synthesis is sold by several vendors, including Applied Biosystems. The preparation of other oligonucleotides such as phosphorothioates and alkylated derivatives is also well known to those of skill in the art.

b) Ribozymes

In addition to antisense polynucleotides, ribozymes can be used to target and inhibit transcription of ZNF217. A ribozyme is an RNA molecule that catalytically cleaves other RNA molecules. Different kinds of ribozymes have been described, including group I ribozymes, hammerhead ribozymes, hairpin ribozymes, RNAse P, and axhead ribozymes (see, e.g., Castanotto et al., 1994, Adv. in Pharmacology 25:289-317 for a general review of the properties of different ribozymes).

The general features of hairpin ribozymes are described, e.g., in Hampel et al., 1990, Nucl. Acids Res., 18:299-304; Hampel et al.,1990, European Patent Publication No. 0 360 257; U.S. Pat. No. 5,254,678. Methods of preparing are well known to those of skill in the art (see, e.g., Wong-Staal et al., WO 94/26877; Ojwang et al., 1993, Proc. Natl. Acad. Sci. USA, 90:6340-6344; Yamada et al., 1994, Human Gene Therapy 1:39-45; Leavitt et al., 1995, Proc. Natl. Acad. Sci. USA, 92:699-703; Leavitt et al., 1994, Human Gene Therapy 5:1151-120; and Yamada et al., 1994, Virology 205:121-126).

c) siRNA

In certain embodiments, ZNF217 activity is downregulated, or entirely inhibited, by the use of siRNA. See, e.g., WO0244321, siRNA refers to a nucleic acid that forms a double stranded RNA, which double stranded RNA has the ability to reduce or inhibit expression of a gene or target gene when the siRNA expressed in the same cell as the gene or target gene. siRNA thus encompasses the double stranded RNA formed by the complementary strands. The complementary portions of the siRNA that hybridize to form the double stranded molecule typically have substantial or complete identity. In one embodiment, an siRNA refers to a nucleic acid that has substantial or complete identity to a target gene and forms a double stranded siRNA. The sequence of the siRNA can correspond to the full length target gene, or a subsequence thereof. Typically, the siRNA is at least about 15-50 nucleotides in length (e.g., each complementary sequence of the double stranded siRNA is 15-50 nucleotides in length, and the double stranded siRNA is about 15-50 base pairs in length, preferable about preferably about 20-30 base nucleotides, preferably about 20-25 nucleotides in length, e.g., 20, 21, 22, 23, 24, 25, 26, 27, 28, 29, or 30 nucleotides in length. siRNAs can be introduced into animals according to any methods, including those of, e.g., U.S. Applications 2002/0132788 and 2002/0173478.

2. Inhibitors of ZNF217 Polypeptide Activity

ZNF217 activity can also be decreased by the addition of an inhibitor of the ZNF217 polypeptide. This can be accomplished in any of a number of ways, including by providing a dominant negative ZNF217 polypeptide, e.g., a form of ZNF217 that itself has no activity and which, when present in the same cell as a functional ZNF217, reduces or eliminates the ZNF217 activity of the functional ZNF217. Design of dominant negative forms is well known to those of skill and is described, e.g., in Herskowitz,1987, Nature, 329:219-22. Also, inactive polypeptide variants (muteins) can be used, e.g., by screening for the ability to inhibit ZNF217 activity. Methods of making muteins are well known to those of skill (see, e.g., U.S. Pat. Nos. 5,486,463, 5,422,260, 5,116,943, 4,752,585, 4,518,504). In addition, any small molecule, e.g., any peptide, amino acid, nucleotide, lipid, carbohydrate, or any other organic or inorganic molecule can be screened for the ability to bind to or inhibit ZNF217 activity, as described below.

D. Modulators and Binding Compounds

The compounds tested as modulators of a ZNF217 protein can be any small chemical compound, or a biological entity, such as a protein, sugar, nucleic acid or lipid. Typically, test compounds will be small chemical molecules and peptides. Essentially any chemical compound can be used as a potential modulator or binding compound in the assays of the invention, although most often compounds can be dissolved in aqueous or organic (especially DMSO-based) solutions. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs, Switzerland) and the like.

In some embodiments, the small molecule ZNF217 inhibitor is triciribine, triciribine phosphate (TCN-P), triciribine 5′-phosphate (TCN-P), and the DMF adduct of triciribine (TCN-DMF). See, e.g., U.S. Pat. No. 6,413,944. TCN may be synthesized as described in Tetrahedron Letters, vol. 49, pp. 4757-4760 (1971), which is incorporated herein by reference. TCN-P may be prepared as described in U.S. Pat. No. 4,123,524, which is incorporated herein by reference. TCN-DMF is described in INSERM, vol. 81, pp. 37-82 (1978).

In some embodiments, the small molecule is selected from the following list or analogs thereof: bis(2-Nitrophenyl)sulfilimine (NSC number 645984); 3-(4-Fluorophenyl)-3-(4-hydroxy- 2-methylphenyl)phthalide (NSC number 682335); (5H-Benzocyclohepten-5-one, 4-(acetyloxy)-6,6-dibro) (NSC number 624771); and N,N-dimethyl-3-((4-pyridinylmethyl)imino)-3H-1,2,4-dithiazol-5-amine hydrobromide (NSC number 661112). These molecules are from the National Cancer Institute chemical depository.

In one preferred embodiment, high throughput screening methods involve providing a combinatorial chemical or peptide library containing a large number of potential therapeutic compounds (potential modulator or binding compounds). Such “combinatorial chemical libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, 1991, Int. J. Pept. Prot. Res. 37:487-493 and Houghton et al., 1991, Nature 354:84-88). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., 1993, Proc. Nat. Acad. Sci. USA 90:6909-6913), vinylogous polypeptides (Hagihara et al., 1992, J. Amer. Chem. Soc. 114:6568), nonpeptidal peptidomimetics with glucose scaffolding (Hirschmann et al., 1992, J. Amer. Chem. Soc. 114:9217-9218), analogous organic syntheses of small compound libraries (Chen et al., 1994, J. Amer. Chem. Soc. 116:2661), oligocarbamates (Cho et al., 1993, Science 261:1303), and/or peptidyl phosphonates (Campbell et al., 1994, J. Org. Chem. 59:658), nucleic acid libraries (see Ausubel, Berger and Sambrook, all supra), peptide nucleic acid libraries (see, e.g., U.S. Pat. No. 5,539,083), antibody libraries (see, e.g., Vaughn et al., 1996, Nature Biotechnology, 14:309-314 and PCT/US96/10287), carbohydrate libraries (see, e.g., Liang et al., 1996, Science, 274:1520-1522 and U.S. Pat. No. 5,593,853), small organic molecule libraries (see, e.g., benzodiazepines, Baum, 1993, C&EN, Jan 18, page 33; isoprenoids, U.S. Pat. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Tripos, Inc., St. Louis, Mo., 3D Pharmaceuticals, Exton, Pa., Martek Biosciences, Columbia, Md., etc.).

1. Solid State and Soluble High Throughput Assays

In one embodiment, the invention provides soluble assays using molecules such as an N-terminal or C-terminal domain either alone or covalently linked to a heterologous protein to create a chimeric molecule. In another embodiment, the invention provides solid phase based in vitro assays in a high throughput format, where a domain, chimeric molecule, ZNF217 protein, or cell or tissue expressing a ZNF217 protein is attached to a solid phase substrate.

In the high throughput assays of the invention, it is possible to screen up to several thousand different modulators in a single day. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (e.g., 96) modulators. If 1536 well plates are used, then a single plate can easily assay from about 100 to about 1500 different compounds. It is possible to assay several different plates per day; assay screens for up to about 6,000-20,000 different compounds is possible using the integrated systems of the invention. More recently, microfluidic approaches to reagent manipulation have been developed.

The molecule of interest can be bound to the solid state component, directly or indirectly, via covalent or non covalent linkage, e.g., via a tag. The tag can be any of a variety of components. In general, a molecule which binds the tag (a tag binder) is fixed to a solid support, and the tagged molecule of interest is attached to the solid support by interaction of the tag and the tag binder.

A number of tags and tag binders can be used, based upon known molecular interactions well described in the literature. For example, where a tag has a natural binder, for example, biotin, protein A, or protein G, it can be used in conjunction with appropriate tag binders (avidin, streptavidin, neutravidin, the Fc region of an immunoglobulin, etc.) Antibodies to molecules with natural binders such as biotin are also widely available and appropriate tag binders; see, SIGMA Immunochemicals 1998 catalogue SIGMA, St. Louis Mo.).

Similarly, any haptenic or antigenic compound can be used in combination with an appropriate antibody to form a tag/tag binder pair. Thousands of specific antibodies are commercially available and many additional antibodies are described in the literature. For example, in one common configuration, the tag is a first antibody and the tag binder is a second antibody which recognizes the first antibody.

Synthetic polymers, such as polyurethanes, polyesters, polycarbonates, polyureas, polyamides, polyethyleneimines, polyarylene sulfides, polysiloxanes, polyimides, and polyacetates can also form an appropriate tag or tag binder. Many other tag/tag binder pairs are also useful in assay systems described herein, as would be apparent to one of skill upon review of this disclosure.

Common linkers such as peptides, polyethers, and the like can also serve as tags, and include polypeptide sequences, such as poly-Gly sequences of between about 5 and 200 amino acids (SEQ ID NO:3). Such flexible linkers are known to persons of skill in the art. For example, poly(ethylene glycol) linkers are available from Shearwater Polymers, Inc. Huntsville, Ala. These linkers optionally have amide linkages, sulfhydryl linkages, or heterofunctional linkages.

Tag binders are fixed to solid substrates using any of a variety of methods currently available. Solid substrates are commonly derivatized or functionalized by exposing all or a portion of the substrate to a chemical reagent which fixes a chemical group to the surface which is reactive with a portion of the tag binder. For example, groups which are suitable for attachment to a longer chain portion would include amines, hydroxyl, thiol, and carboxyl groups. Aminoalkylsilanes and hydroxyalkylsilanes can be used to functionalize a variety of surfaces, such as glass surfaces. The construction of such solid phase biopolymer arrays is well described in the literature. See, e.g., Merrifield, 1993, J. Am. Chem. Soc. 85:2149-2154 (describing solid phase synthesis of, e.g., peptides); Geysen et al., 1987, J. Immun. Meth. 102:259-274 (describing synthesis of solid phase components on pins); Frank & Doring, 1988, Tetrahedron 44:60316040 (describing synthesis of various peptide sequences on cellulose disks); Fodor et al., 1991, Science, 251:767-777; Sheldon et al., 1993, Clinical Chemistry 39:718-719; and Kozal et al., 1996, Nature Medicine 2:753759 (all describing arrays of biopolymers fixed to solid substrates). Nonchemical approaches for fixing tag binders to substrates include other common methods, such as heat, cross-linking by UV radiation, and the like.

2. Rational Drug Design Assays

Yet another assay for compounds that modulate ZNF217 protein activity involves computer assisted drug design, in which a computer system is used to generate a three-dimensional structure of a ZNF217 protein based on the structural information encoded by its amino acid sequence. The input amino acid sequence interacts directly and actively with a pre-established algorithm in a computer program to yield secondary, tertiary, and quaternary structural models of the protein. The models of the protein structure are then examined to identify regions of the structure that have the ability to bind. These regions are then used to identify compounds that bind to the protein.

The three-dimensional structural model of the protein is generated by entering protein amino acid sequences of at least 10 amino acid residues or corresponding nucleic acid sequences encoding a ZNF217 polypeptide into the computer system. The nucleotide sequence encoding the polypeptide, or the amino acid sequence thereof, and conservatively modified versions thereof, of the naturally occurring ZFN217 gene sequence. The amino acid sequence represents the primary sequence or subsequence of the protein, which encodes the structural information of the protein. At least 10 residues of the amino acid sequence (or a nucleotide sequence encoding 10 amino acids) are entered into the computer system from computer keyboards, computer readable substrates that include, but are not limited to, electronic storage media (e.g., magnetic diskettes, tapes, cartridges, and chips), optical media (e.g., CD ROM), information distributed by internet sites, and by RAM. The three-dimensional structural model of the protein is then generated by the interaction of the amino acid sequence and the computer system, using software known to those of skill in the art.

The amino acid sequence represents a primary structure that encodes the information necessary to form the secondary, tertiary and quaternary structure of the protein of interest. The software looks at certain parameters encoded by the primary sequence to generate the structural model. These parameters are referred to as “energy terms,” and primarily include electrostatic potentials, hydrophobic potentials, solvent accessible surfaces, and hydrogen bonding. Secondary energy terms include van der Waals potentials. Biological molecules form the structures that minimize the energy terms in a cumulative fashion. The computer program is therefore using these terms encoded by the primary structure or amino acid sequence to create the secondary structural model.

The tertiary structure of the protein encoded by the secondary structure is then formed on the basis of the energy terms of the secondary structure. The user at this point can enter additional variables such as whether the protein is membrane bound or soluble, its location in the body, and its cellular location, e.g., cytoplasmic, surface, or nuclear. These variables along with the energy terms of the secondary structure are used to form the model of the tertiary structure. In modeling the tertiary structure, the computer program matches hydrophobic faces of secondary structure with like, and hydrophilic faces of secondary structure with like.

Once the structure has been generated, potential modulator binding regions are identified by the computer system. Three-dimensional structures for potential modulators are generated by entering amino acid or nucleotide sequences or chemical formulas of compounds, as described above. The three-dimensional structure of the potential modulator is then compared to that of the ZNF217 protein to identify compounds that bind to the protein. Binding affinity between the protein and compound is determined using energy terms to determine which compounds have an enhanced probability of binding to the protein.

Computer systems are also used to screen for mutations, polymorphic variants, alleles and interspecies homologs of ZNF217 genes. Such mutations can be associated with disease states or genetic traits. As described above, GeneChip™ and related technology can also be used to screen for mutations, polymorphic variants, alleles and interspecies homologs. Once the variants are identified, diagnostic assays can be used to identify patients having such mutated genes. Identification of the mutated ZNF217 genes involves receiving input of a first nucleic acid or amino acid sequence of the naturally occurring ZNF217 gene, respectively, and conservatively modified versions thereof. The sequence is entered into the computer system as described above. The first nucleic acid or amino acid sequence is then compared to a second nucleic acid or amino acid sequence that has substantial identity to the first sequence. The second sequence is entered into the computer system in the manner described above. Once the first and second sequences are compared, nucleotide or amino acid differences between the sequences are identified. Such sequences can represent allelic differences in various ZNF217 genes, and mutations associated with disease states and genetic traits.

VII. Modulating ZNF217 Activity/Expression to Treat Dieases or Conditions

In numerous embodiments of this invention, a compound, e.g., nucleic acid, polypeptide, or other molecule is administered to a patient, in vivo or ex vivo, to effect a change in ZNF217 activity or expression in the patient. The desired change can be either an increase or a decrease in activity or expression of ZNF217. For example, in a cancer patient with a tumor that exhibits increased levels of ZNF217 relative to normal tissue, it can be desirable to decrease the activity or expression of ZNF217. In other embodiments of the invention, antibodies that block ZNF217 activity or function can be administered to a patient with a ZNF217-expressing tumor to inhibit ZNF217 function at the cell membrane surface and thus inhibit tumor growth, migration, or metastasis. In other patients with diseases associated with decreased activity or expression of ZNF217, it can be desirable to increase the activity or expression of ZNF217.

Compounds that can be administered to a patient include nucleic acids encoding full length ZNF217 polypeptides, or any derivative, fragment, or variant thereof, operably linked to a promoter. Suitable nucleic acids also include inhibitory sequences such as antisense, silencing RNA (siRNA) (e.g., fewer than 30 nucleotides in length) or ribozyme sequences, which can be delivered in, e.g., an expression vector operably linked to a promoter, or can be delivered directly. Also, any nucleic acid that encodes a polypeptide that modulates the expression of ZNF217 can be used. In general, nucleic acids can be delivered to cells using any of a large number of vectors or methods, e.g., retroviral, adenoviral, or adeno-associated virus vectors, liposomal formulations, naked DNA injection, and others. All of these methods are well known to those of skill in the art.

Proteins can also be delivered to a patient to modulate ZNF217 activity. In preferred embodiments, a polyclonal or monoclonal antibody that specifically binds to ZNF217 will be delivered. In addition, any polypeptide that interacts with and/or modulates ZNF217 activity can be used, e.g., a polypeptide that is identified using the presently described assays. In addition, polypeptides that affect ZNF217 expression can be used.

Further, any compound that is found to or designed to interact with and/or modulate the activity of ZNF217 can be used. For example, any compound that is found, using the methods described herein, to bind to or modulate the activity of ZNF217 can be used.

Any of the above-described molecules can be used to increase or decrease the expression or activity of ZNF217, or to otherwise affect the properties and/or behavior of ZNF217 polypeptides or polynucleotides, e.g., stability, intracellular localization, interactions with other intracellular or extracellular moieties, and the like.

A. Pharmaceutical Compositions

Administration of any of the present molecules of the invention can be achieved by any of the routes normally used for introducing or bringing a modulator compound into ultimate contact with the tissue to be treated. The modulators are administered in any suitable manner, optionally with pharmaceutically acceptable carriers. Suitable methods of administering such modulators are available and well known to those of skill in the art, and, although more than one route can be used to administer a particular composition, a particular route can often provide a more immediate and more effective reaction than another route.

The invention provides pharmaceutical compositions comprising one or a combination of ZFN217 modulators formulated together with a pharmaceutically acceptable carrier.

1. Effective Dosages

Dosage regimens are adjusted to provide the optimum desired response (e.g., a therapeutic response). For example, a single bolus can be administered, several divided doses can be administered over time or the dose can be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

Examples of pharmaceutically-acceptable antioxidants include:(1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Regardless of the route of administration selected, the compounds of the present invention, which can be used in a suitable hydrated form, and/or the pharmaceutical compositions of the present invention, are formulated into pharmaceutically acceptable dosage forms by conventional methods known to those of skill in the art.

Actual dosage levels of the active ingredients in the pharmaceutical compositions of the present invention can be varied so as to obtain an amount of the active ingredient which is effective to achieve the desired therapeutic response for a particular patient, composition, and mode of administration, without being toxic to the patient. The selected dosage level depends upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors.

A physician or veterinarian can start doses of the compounds of the invention employed in the pharmaceutical composition at levels lower than that required to achieve the desired therapeutic effect and gradually increase the dosage until the desired effect is achieved. In general, a suitable daily dose of a compositions of the invention is that amount of the compound which is the lowest dose effective to produce a therapeutic effect. Such an effective dose generally depends upon the factors described above. It is preferred that administration be intravenous, intramuscular, intraperitoneal, or subcutaneous, or administered proximal to the site of the target. If desired, the effective daily dose of a therapeutic compositions can be administered as two, three, four, five, six or more sub-doses administered separately at appropriate intervals throughout the day, optionally, in unit dosage forms. While it is possible for a compound of the present invention to be administered alone, it is preferable to administer the compound as a pharmaceutical formulation (composition).

Effective doses of the compositions of the present invention, for the treatment of diseases described herein vary depending upon many different factors, including means of administration, target site, physiological state of the patient, whether the patient is human or an animal, other medications administered, and whether treatment is prophylactic or therapeutic. Treatment dosages need to be titrated to optimize safety and efficacy.

For administration with an antibody, the dosage ranges from about 0.0001 to 100 mg/kg, and more usually 0.01 to 5 mg/kg, of the host body weight. For example dosages can be 1 mg/kg body weight or 10 mg/kg body weight or within the range of 1-10 mg/kg. An exemplary treatment regime entails administration once per every two weeks or once a month or once every 3 to 6 months. In some methods, two or more monoclonal antibodies with different binding specificities are administered simultaneously, in which case the dosage of each antibody administered falls within the ranges indicated. Antibody is usually administered on multiple occasions. Intervals between single dosages can be weekly, monthly or yearly. Intervals can also be irregular as indicated by measuring blood levels of antibody to ZFN217 in the patient. In some methods, dosage is adjusted to achieve a plasma antibody concentration of 1-1000 μg/ml and in some methods 25-300 μg/ml. Alternatively, antibody can be administered as a sustained release formulation, in which case less frequent administration is required. Dosage and frequency vary depending on the half-life of the antibody in the patient. In general, human antibodies show the longest half life, followed by humanized antibodies, chimeric antibodies, and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patent can be administered a prophylactic regime.

Doses for nucleic acids encoding immunogens range from about 10 ng to 1 g, 100 ng to 100 mg, 1 μg to 10 mg, or 30-300 μg DNA per patient. Doses for infectious viral vectors vary from 10-100, or more, virions per dose.

Some compounds of the invention can be formulated to ensure proper distribution in vivo. For example, the blood-brain barrier (BBB) excludes many highly hydrophilic compounds. To ensure that the therapeutic compounds of the invention cross the BBB (if desired), they can be formulated, for example, in liposomes. For methods of manufacturing liposomes, See, e.g., U.S. Pat. Nos. 4,522,811; 5,374,548; and 5,399,331. The liposomes can comprise one or more moieties which are selectively transported into specific cells or organs, thus enhance targeted drug delivery (See, e.g., V. V. Ranade, 1989, J. Clin. Pharmacol. 29:685). Exemplary targeting moieties include folate or biotin (See, e.g., U.S. Pat. No. 5,416,016 to Low et al.); mannosides (Umezawa et al., 1988, Biochem. Biophys. Res. Commun. 153:1038); antibodies (P. G. Bloeman et al., 1995, FEBS Lett. 357:140; M. Owais et al., 1995, Antimicrob. Agents Chemother. 39:180); surfactant protein A receptor (Briscoe et al., 1995, Am. J. Physiol. 1233:134), different species of which can comprise the formulations of the inventions, as well as components of the invented molecules; p120 (Schreier et al., 1994, J. Biol. Chem. 269:9090); See also K. Keinanen; M. L. Laukkanen, 1994, FEBS Lett. 346:123; J. J. Killion; I. J. Fidler, 1994, Immunomethods 4:273. In some methods, the therapeutic compounds of the invention are formulated in liposomes; in a more preferred embodiment, the liposomes include a targeting moiety. In some methods, the therapeutic compounds in the liposomes are delivered by bolus injection to a site proximal to the tumor or infection. The composition should be fluid to the extent that easy syringability exists. It should be stable under the conditions of manufacture and storage and should be preserved against the contaminating action of microorganisms such as bacteria and fungi.

For therapeutic applications, the pharmaceutical compositions are administered to a patient suffering from established disease in an amount sufficient to arrest or inhibit further development or reverse or eliminate, the disease, its symptoms or biochemical markers. For prophylactic applications, the pharmaceutical compositions are administered to a patient susceptible or at risk of a disease in an amount sufficient to delay, inhibit or prevent development of the disease, its symptoms and biochemical markers. An amount adequate to accomplish this is defined as a “therapeutically-” or “prophylactically-effective dose.” Dosage depends on the disease being treated, the subject's size, the severity of the subject's symptoms, and the particular composition or route of administration selected. Specifically, in treatment of tumors, a “therapeutically effective dosage” can inhibit tumor growth by at least about 20%, or at least about 40%, or at least about 60%, or at least about 80% relative to untreated subjects. The ability of a compound to inhibit cancer can be evaluated in an animal model system predictive of efficacy in human tumors. Alternatively, this property of a composition can be evaluated by examining the ability of the compound to inhibit by conventional assays in vitro. A therapeutically effective amount of a therapeutic compound can decrease tumor size, or otherwise ameliorate symptoms in a subject.

The composition should be sterile and fluid to the extent that the composition is deliverable by syringe. In addition to water, the carrier can be an isotonic buffered saline solution, ethanol, polyol (for example, glycerol, propylene glycol, and liquid polyetheylene glycol, and the like), and suitable mixtures thereof. Proper fluidity can be maintained, for example, by use of coating such as lecithin, by maintenance of required particle size in the case of dispersion and by use of surfactants. In many cases, it is preferable to include isotonic agents, for example, sugars, polyalcohols such as mannitol or sorbitol, and sodium chloride in the composition. Long-term absorption of the injectable compositions can be brought about by including in the composition an agent which delays absorption, for example, aluminum monostearate or gelatin.

When the active compound is suitably protected, as described above, the compound can be orally administered, for example, with an inert diluent or an assimilable edible carrier.

2. Routes of Administration

Pharmaceutical compositions of the invention also can be administered in combination therapy, i.e., combined with other agents. For example, in treatment of cancer, the combination therapy can include a composition of the present invention with at least one anti-tumor agent or other conventional therapy, such as radiation treatment.

Pharmaceutically acceptable carriers includes solvents, dispersion media, coatings, antibacterial and antifungal agents, isotonic and absorption delaying agents, and the like that are physiologically compatible. The carrier can be suitable for intravenous, intramuscular, subcutaneous, parenteral, spinal or epidermal administration (e.g., by injection or infusion). Depending on the route of administration, the active compound, i.e., antibody, bispecific and multispecific molecule, can be coated in a material to protect the compound from the action of acids and other natural conditions that can inactivate the compound.

A “pharmaceutically acceptable salt” refers to a salt that retains the desired biological activity of the parent compound and does not impart any undesired toxicological effects (See, e.g., Berge, S. M., et al., 1977, J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

A composition of the present invention can be administered by a variety of methods known in the art. The route and/or mode of administration vary depending upon the desired results. The active compounds can be prepared with carriers that protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are described by e.g., SUSTAINED AND CONTROLLED RELEASE DRUG DELIVERY SYSTEMS, J. R. Robinson, Ed., 1978, Marcel Dekker, Inc., New York.

To administer a compound of the invention by certain routes of administration, it can be necessary to coat the compound with, or co-administer the compound with, a material to prevent its inactivation. For example, the compound can be administered to a subject in an appropriate carrier, for example, liposomes, or a diluent. Pharmaceutically acceptable diluents include saline and aqueous buffer solutions. Liposomes include water-in-oil-in-water CGF emulsions as well as conventional liposomes (Strejan et al., 1984, J. Neuroimmunol. 7:27).

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile, substantially isotonic, and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it is preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof. Therapeutic compositions can also be administered with medical devices known in the art. For example, in a preferred embodiment, a therapeutic composition of the invention can be administered with a needleless hypodermic injection device, such as the devices disclosed in, e.g., U.S. Pat. Nos. 5,399,163, 5,383,851, 5,312,335, 5,064,413, 4,941,880, 4,790,824, or 4,596,556. Examples of implants and modules useful in the present invention include: U.S. Pat. No. 4,487,603, which discloses an implantable micro-infusion pump for dispensing medication at a controlled rate; U.S. Pat. No. 4.,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Many other such implants, delivery systems, and modules are known.

3. Formulation

For the therapeutic compositions, formulations of the present invention include those suitable for oral, nasal, topical (including buccal and sublingual), rectal, vaginal and/or parenteral administration. The formulations can conveniently be presented in unit dosage form and can be prepared by any methods known in the art of pharmacy. The amount of active ingredient which can be combined with a carrier material to produce a single dosage form vary depending upon the subject being treated, and the particular mode of administration. The amount of active ingredient which can be combined with a carrier material to produce a single dosage form generally be that amount of the composition which produces a therapeutic effect. Generally, out of one hundred per cent, this amount range from about 0.01% to about 99% of active ingredient, from about 0.1% to about 70%, or from about 1% to about 30%.

Formulations of the present invention which are suitable for vaginal administration also include pessaries, tampons, creams, gels, pastes, foams or spray formulations containing such carriers as are known in the art to be appropriate. Dosage forms for the topical or transdermal administration of compositions of this invention include powders, sprays, ointments, pastes, creams, lotions, gels, solutions, patches and inhalants. The active compound can be mixed under sterile conditions with a pharmaceutically acceptable carrier, and with any preservatives, buffers, or propellants which can be required.

The phrases “parenteral administration” and “administered parenterally” mean modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Examples of suitable aqueous and nonaqueous carriers which can be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions can also contain adjuvants such as preservatives, wetting agents, emulsifying agents and dispersing agents. Prevention of presence of microorganisms can be ensured both by sterilization procedures, supra, and by the inclusion of various antibacterial and antifungal agents, for example, paraben, chlorobutanol, phenol sorbic acid, and the like. It can also be desirable to include isotonic agents, such as sugars, sodium chloride, and the like into the compositions. In addition, prolonged absorption of the injectable pharmaceutical form can be brought about by the inclusion of agents which delay absorption such as aluminum monostearate and gelatin.

When the compounds of the present invention are administered as pharmaceuticals, to humans and animals, they can be given alone or as a pharmaceutical composition containing, for example, 0.01 to 99.5% (or 0.1 to 90%) of active ingredient in combination with a pharmaceutically acceptable carrier.

The pharmaceutical compositions are generally formulated as sterile, substantially isotonic and in full compliance with all Good Manufacturing Practice (GMP) regulations of the U.S. Food and Drug Administration.

VIII. Diagnosing Cancer

The present invention provides numerous methods for diagnosing any of a number of types of cancer, e.g., determining whether or not a patient has cancer, whether or not a biological sample contains cancerous cells, estimating the likelihood of a patient developing cancer, and monitoring the efficacy of anti-cancer treatment in a patient with cancer. Such methods are based on the discovery that cancer cells have an elevated level of ZNF217 polynucleotide (i.e., gene copy number and/or mRNA) and polypeptide level. Accordingly, by determining whether or not a cell contains elevated levels of ZNF217 polynucleotide or polypeptide, it is possible to determine whether or not the cell is cancerous. Further, the presence of cancerous cells can be determined indirectly, i.e., in certain embodiments a biological sample that does not itself contain cancerous cells, but which has been taken from an animal with cancerous cells elsewhere in its body, may contain elevated levels of ZNF217 reflecting the presence of the cancerous cells.

A. Detecting a Cancer

In numerous embodiments of the present invention, the level and/or presence or ZNF217 polynucleotide or polypeptide (or allelic variants thereof) will be detected in a biological sample, thereby detecting the presence or absence of cancerous cells in the biological sample, or, in certain embodiments, in the patient from which the biological sample was removed. In preferred embodiments, the biological sample will comprise a tissue sample from a tissue suspected of containing cancerous cells. For example, in a woman suspected of having breast cancer, breast tissue is removed. Often, such methods will be used in conjunction with additional diagnostic methods, e.g., detection of other cancer markers, mammography, and the like. In other embodiments, a tissue sample known to contain cancerous cells, e.g., from a tumor, will be detected for ZNF217 levels to determine information about the cancer, e.g., the efficacy of certain treatments, the survival expectancy of the animal, and the like.

The amount of ZNF217 polynucleotide or polypeptide that will indicate the presence of a cancer will depend on numerous factors, including the type of cancer, the age, sex, medical history, and the like, of the patient, the cell type, the assay format, and the like In preferred embodiments, a level of ZNF217 in a biological sample will not be quantified or directly compared with a control sample, but will rather be detected relative to a “diagnostic presence” of ZNF217, wherein a “diagnostic presence” refers to an amount of ZNF217 polynucleotide or polypeptide that indicates the presence of cancer, or indicates a likelihood of cancer, in the patient from which the sample was taken. Preferably, a “diagnostic presence” will be detectable in a simple assay giving a positive or negative result, where a positive “detection” of a “diagnostic presence” of ZNF217 polynucleotide or polypeptide indicates the presence of cancer in the patient.

The ZNF217 level need not be quantified for a “diagnostic presence” to be detected, merely any method of determining whether ZNF217 is present at levels higher than in a normal, cancer-free cell, sample, or mamml. In addition, a “diagnostic presence” does not refer to any absolute quantity of ZNF217, but rather on an amount that, depending on the biological sample, cell type, assay conditions, medical condition of the patient, and the like, is sufficient to distinguish the level in a cancerous, or pre-cancerous sample, from a normal, cancer-free sample.

Such methods can be practiced regardless of whether any ZNF217 polynucleotide or polypeptide is normally present, or “expected” to be present, in a particular control sample. For example, ZNF217 may not be expressed in certain cell types, resulting in a complete absence of ZNF217 in a control biological sample consisting of such cell types. For such biological sample, a “diagnostic presence” refers to any detectable amount of ZNF217, using any assay. In other tissues, however, there may be a detectable level of ZNF217 present in normal, cancer-free cells, and a “diagnostic presence” represents a level that is higher than the normal level, preferably representing a “statistically significant” increase over the normal level. Often, as discussed supra, a “diagnostic presence” of ZNF217 polynucleotide, polypeptide, and/or protein activity in a biological sample will be at least about 1.5, 2, 5, 10, or more fold greater than a level expected in a sample taken from a normal, cancer-free patient.

Further, the present methods can be used to assess the efficacy of a course of treatment. For example, in a patient with cancer from which a biological sample has been found to contain an elevated amount of ZNF217 polynucleotide or polypeptide, the efficacy of an anti-cancer treatment can be assessed by monitoring, over time, ZNF217 levels. For example, a reduction in ZNF217 polynucleotide or polypeptide levels in a biological sample taken from a patient following a treatment, compared to a level in a sample taken from the patient before, or earlier in, the treatment, indicates efficacious treatment.

B. Determining a Prognosis

The level of ZNF217 or allelic variants thereof can be used to determine the prognosis of a patient with cancer. For example, if cancer is detected using a technique other than by detecting ZNF217, e.g., tissue biopsy, then the presence or absence of ZNF217 can be used to determine the prognosis for the patient, i.e., an elevated level of ZNF217 will indicate a reduced survival expectancy in the patient compared to in a patient with cancer but with a normal level of ZNF217. As used herein, “survival expectancy” refers to a prediction regarding the severity, duration, or progress of a disease, condition, or any symptom thereof. In a preferred embodiment, an increased level, a diagnostic presence, or a quantified level, of ZNF217 is statistically correlated with the observed progress of a disease, condition, or symptom in a large number of patients, thereby providing a database wherefrom a statistically-based prognosis can be made in view of any detected level or presence of ZNF217. For example, in a particular type of patient, i.e., a human of a particular age, gender, medical condition, medical history, and the like, a detection of a level of ZNF217 that is, e.g., 2 fold higher than a control level may indicate, e.g., a 10% reduced survival expectancy in the human compared to in a similar human with a normal level of ZNF217, based on a previous study of the level of ZNF217 in a large number of similar patients whose disease progression was observed and recorded.

C. Determining a Preferred Course of Treatment

The present methods can be used to determine the optimal course of treatment in a patient with cancer. For example, the presence of an elevated level of ZNF217 can indicate a reduced survival expectancy of a patient with cancer, thereby indicating a more aggressive treatment for the patient. In addition, a correlation can be readily established between levels of ZNF217, or the presence or absence of a diagnostic presence of ZNF217, and the relative efficacy of one or another anti-cancer agent. Such analyses can be performed, e.g., retrospectively, i.e., by detecting ZNF217 levels in samples taken previously from patients that have subsequently undergone one or more types of anti-cancer therapy, and correlating the ZNF217 levels with the known efficacy of the treatment.

In numerous embodiments, levels of ZNF217 polynucleotides or polypeptides in tumor cells of a patient, e.g., as detected by immunoassay using anti-ZNF217 antibodies, are used to guide the selection of an anti-cancer treatment based on the effects of the treatment ZNF217 or its activity. In preferred embodiments, a detection of an elevated or diagnostic level of ZNF217 indicates the beneficial use of a treatment that inhibits the activity ZFN217 or ZNF217 allelic variants thereof.

IX. Treating Cancer

The present invention provides numerous methods for treating a patient with cancer. In addition to allowing the determination of an optimal treatment for a patient with cancer, as described supra, methods are provided for treating a cancer by inhibiting the growth, proliferation, or metastatic production of cells within the patient, e.g., cancer cells. Typically, the methods are directed at reducing the level of ZNF217 polypeptides, polynucleotides, or protein activity in a cancerous cell. It will be appreciated that more than one of the methods described infra can be performed on a given subject or patient, and can also be administered in conjunction with one or more traditional, well known anti-cancer therapies, e.g., chemotherapy, radiation therapy, surgery, hormone therapy, immunotherapy, and the like.

According to the present invention, a “method of treating cancer” refers to a procedure or course of action that is designed to reduce or eliminate the number of cancer cells in an animal, or to alleviate the symptoms of a cancer. “A method of treating cancer” does not necessarily mean that the cancer cells will, in fact, be eliminated, that the number of cells will, in fact, be reduced, or that the symptoms of a cancer will, in fact, be alleviated. Often, a method of treating cancer will be performed even with a low likelihood of success, but which, given the medical history and estimated survival expectancy of an subject or patient, is nevertheless deemed an overall beneficial course of action.

In certain embodiments, the present invention provides methods for treating cancer when a diagnostic presence or increased level is detected, applying one or more anti-cancer therapies, including, but not limited to, chemotherapy, radiation therapy, surgery, immunotherapy, hormone therapy, and gene therapy. In certain embodiments, the ZNF217 modulators can be used effectively alone or in combination with one or more additional anti-cancer therapies as discussed herein.

One commonly applied anti-cancer therapy is chemotherapy, i.e., the administration of chemical compounds to a patient with cancer that is aimed at killing or reducing the number of cancer cells within the patient. Generally, chemotherapeutic agents arrest the growth of or kill cells that are dividing or growing, such as cancer cells. Examples of chemotherapeutic agents include, but are not limited to, genistein, taxol, busulfan, cisplatin, cyclophosphamide (cytoxan), dacarbazine, ifosfamide, mechlorethamine, melphalan, carmustine, lomustine, 5-fluorouracil, methotrexate, gemcitabine, cytarabine (Ara-C), fludarabine, bleomycin, dactinomycin, daunorubicin, doxorubicin (Adriamycin), idarubicin, paclitaxel, docetaxel, etoposide, vinblastine, vincristine, vinorelbine, L-asparaginase, amsacrine, tretinoin, prednisone and dexamethasone.

In some embodiments, preferred chemotherapeutic agents are drugs that promote apoptosis. In other embodiments, the chemotherapeutic drug is a topoisomerase inhibitor. In other embodiments, the chemotherapeutic drug is doxorubicin.

Another commonly applied anti-cancer therapy is radiation therapy, wherein radioactivity is administered to a patient with cancer. Radiation kills or inhibits the growth of dividing cells, such as cancer cells. The administration of radiation can be from an external source (e.g., a gamma source, a proton source, a molecular beam source, and the like.), or can be through an implantable radioactive material, or a radioactive molecule such as an antibody.

In numerous embodiments, a tissue found to be cancerous using the present methods will be removed using surgery, i.e., the direct removal or ablation of cells, e.g., cancer cells, from a patient. Most often, the cancer cells will be in the form of a tumor (e.g., a mammary tumor), which is removed from the patient. The surgical methods can involve removal of healthy as well as cancerous tissue.

Hormone therapy can also be used to treat cancers, e.g., breast cancer. For example, compounds can be administered to a patient that counteract or inhibit hormones, such as estrogen or androgen, that have a mitogenic effect on cells and which often act to increase the cancerous properties of cancer cells in vivo. Hormone therapy can also include methods of reducing or eliminating the production of hormones in an patient or subject, e.g., the surgical removal of ovaries in an patient or subject to prevent estrogen production.

In certain embodiments, immunotherapy will be used to treat a cancer following a diagnosis based on detection of high-level amplification ZNF217, i.e., methods of enhancing the ability of an patient's immune system to destroy cancer cells within the patient or subject. Numerous such methods are well known to those of skill in the art. This can involve the treatment with polyclonal or monoclonal antibodies (e.g., Herceptin) that bind to particular molecules located on, produced by, or indicative of, tumor cells. Immunotherapeutic methods are well know to those of skill in the art (see, e.g., Pastan et al., 1992, Ann. Rev. Biochem., 61:331-354, Brinkman and Pastan, 1994, Biochimica Biphysica Acta, 1198:27-45).

In other embodiments, gene therapy will be used to treat a cancer diagnosed based on a detection of ZNF217. In such embodiments, a nucleic acid is introduced into cells, e.g., cancer cells, to provide treatment for the cancer. For example, tumor suppressor genes that are often missing or mutated in a cancer cell, e.g., p53, RB, p21, p16, and others, can be replaced or overexpressed by introducing a nucleic acid encoding a functional gene into the cells. In addition, genes whose overexpression or increased activity contributes to cancer, e.g., ras, telomerase, and the like, can be inhibited by any of a number of methods, including, but not limited to, antisense, siRNA, ribozymes, and polynucleotides encoding dominant negative forms or other inhibiting polypeptides. Such nucleic acids can be delivered using any of a variety of methods, e.g., liposomal formulations, viral vectors, naked DNA injection, and the like, and can be performed in vivo or ex vivo.

Such gene therapy procedures have been used to correct acquired and inherited genetic defects, cancer, and other diseases in a number of contexts. The ability to express artificial genes in humans facilitates the prevention and/or cure of many important human diseases, including many diseases which are not amenable to treatment by other therapies (for a review of gene therapy procedures, see Anderson, 1992, Science 256:808-813; Nabel & Felgner, 1993, TIBTECH 11:211-217; Mitani & Caskey, 1993 TIBTECH 11:162-166; Mulligan, 1993, Science 926-932; Dillon, 1993 TIBTECH 11:167-175; Miller, 1992, Nature 357:455-460; Van Brunt, 1998, Biotechnology 6:1149-1154; Vigne, 1995, Restorative Neurology and Neuroscience 8:35-36; Kremer & Perricaudet, 1995, British Medical Bulletin 51:31-44; Haddada et al., in CURRENT TOPICS IN MICROBIOLOGY AND IMMUNOLOGY (Doerfler & Böhm eds., 1995); and Yu et al., 1994, Gene Therapy 1:13 -26).

The present methods can be used to treat any of a number of types of cancers. In preferred embodiments, epithelial cancers will be diagnosed and/or treated, e.g., breast cancer. Other epithelial cancers include, e.g., ovarian, colorectal, kidney, stomach, bladder, and lung cancers. A cancer at any stage of progression can be detected, such as primary, metastatic, and recurrent cancers. Information regarding numerous types of cancer can be found, e.g., from the American Cancer Society (www.cancer.org), or from, e.g., Wilson et al., 1991, McGraw-Hill, Inc., HARRISON'S PRINCIPLES OF INTERNAL MEDICINE, 12^{th} Ed.

X. Kits

Reagents that specifically hybridize to ZNF217 nucleic acids, such as ZNF217 probes and primers, and ZNF217-specific reagents that specifically bind to or modulate the activity of a ZNF217 protein, e.g., ZNF217 antibodies or other compounds are used to treat ZNF217-associated diseases or conditions.

Nucleic acid assays for detecting the presence of DNA and RNA for a ZNF217 polynucleotide in a sample include numerous techniques known to those skilled in the art, such as Southern analysis, Northern analysis, dot blots, RNase protection, S1 analysis, amplification techniques such as PCR and LCR, and in situ hybridization. In in situ hybridization, for example, the target nucleic acid is liberated from its cellular surroundings so as to be available for hybridization within the cell while preserving the cellular morphology for subsequent interpretation and analysis. The following articles provide an overview of the art of in situ hybridization: Singer et al., 1986, Biotechniques 4:230-250; Haase et al., 1984, METHODS IN VIROLOGY, Vol. VII, pp.189-226; and NUCLEIC ACID HYBRIDIZATION: A PRACTICAL APPROACH (Hames et al., eds. 1987). In addition, a ZNF217 protein can be detected using the various immunoassay techniques described above. The test sample is typically compared to both a positive control (e.g., a sample expressing a recombinant ZNF217 protein) and a negative control.

The present invention also provides for kits for screening for modulators of ZNF217 proteins or nucleic acids. Such kits can be prepared from readily available materials and reagents. For example, such kits can comprise any one or more of the following materials: ZNF217 nucleic acids or proteins, reaction tubes, and instructions for testing ZNF217 activity. Optionally, the kit contains a biologically active ZNF217 protein. For use in diagnostic, research, and therapeutic applications suggested above, kits are also provided by the invention. In the diagnostic and research applications such kits can include any or all of the following: assay reagents, buffers, ZNF217 specific nucleic acids or antibodies, hybridization probes and/or primers, antisense polynucleotides, siRNA, ribozymes, dominant negative ZNF217 polypeptides or polynucleotides, small molecules inhibitors of ZNF217, and the like. A therapeutic product can include sterile saline or another pharmaceutically acceptable emulsion and suspension base as described above.

In addition, the kits can include instructional materials containing directions (i.e., protocols) for the practice of the methods of this invention. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips, and the like), optical media (e.g., CD ROM), and the like. Such media can include addresses to internet sites that provide such instructional materials.

### EXAMPLES

The following examples are provided solely to illustrate in greater detail particular aspects of the disclosed methods, compositions and assays and should not be construed to be limiting in any way.

### Example 1

I. Materials and Methods

Cell Lines and Cell Culture

Hela cells were obtained from American Type Culture Collection (ATCC) and cultured in MED-H-21 with 10% FBS and 100 mg/ml penicillin and streptomycin at 37° C. with 5% CO₂. Hela cells were transfected with a ZNF217-GFP plasmid (Collins, C. et al., 2001, Genome Res 11:1034-42) using Lipofectine (Qiagene) and stable transfectants selected in 500 mg/ml Genemycine (G418). Breast cancer cell lines BT474, MCF7, 600MPE, HBL100, MDA-435, HS578T were cultured as described (Collins, C. et al., 1998, supra).

ZNF217-EGFP Transfections

A full length ZNF217 EGFP fusion cDNA gene (Collins et al., 2001, supra) was transfected into HeLa and HBL100 cells using Fugene 6 (Roche) according to the manufactures protocol. The EGFP positive population were sorted by flow cytometry and medium with 500 μg/ml Geneticin (G418). ZNF217-EGFP mRNA expression in transfected Hela cells was measured by Northern blotting using as 3′ untranslated probe as previously described (Collins et al., 1998, supra). Subcellular localization of the ZNF217-EGFP fusion protein and image acquisition was accomplished using a Zeiss confocal microscopy system.

TRF2 Dominant-Negative Transfections

1.5×10⁵ HeLa and HBL100 cells+/−ZNF217-EGFP were plated in six well plates and cultured as described. On day two a plasmid encoding a TRF2 dominant negative (Karlseder, J. et al., 1999, supra) was transfected as described above and after 24 hours the Fugene 6 transfection reagent was removed and the cells were cultured for 48 hours in fresh culture medium as described for an additional 48 hours, the cells including dead cells in the supernatants were collected and apoptotic cells were detected as described below.

Cellular Growth Rate

1.5×10⁵ HeLa cells and parallel cultures of transfected cells were plated in six well microtiter plates and grown in 3 ml of 10% FB, 100 mg/ml penicillin and streptomycin supplemented DME-H-21 media at 37° C. with 5% CO₂. 500 mg/ml G418 was used to maintain selection of the ZNF217-EGFP plasmid. At 24, 48 and 72 hours cells were harvested, suspended in PBS and counted using trypan blue and a hemocytometer.

Cell Cycle Analysis

Three of 1.5×10⁵ of Hela and ZNF217-GFP transfected Hela cells were plated in 6 well plates and cultured as described above, separately. 24, 48, 72 hours later, the cells were tripsinized, washed with PBS and prepared single cell suspension in PBS buffer with 3% FBS. Then the cells were fixed with cold 80% ethenol for 30 minutes. After washing twice with PBS, the cells were suspensed in 0.5 ml of 50 μg/ml propidium iodide (PI) PBS staining solution and 300 μl of 2 mg/ml RNase A and incubated 1 hour at room temperature at dark room. Cell Cycle was analyzed with flow cytometry following standard methods and procedures.

Cell Cycle Measurements

On the first day (0 hr), plated 3 of 1.5×10⁵ of Hela cells and ZNF217-Hela cells in the 6 well plates, separately. After growing 24, 48, 72 hours later, harvested cells and prepared single cell suspension in PBS+3%FBS buffer. Washed cells twice and resuspend at 1-2×10⁶ cells/ml. Aliquot 1×10⁶ cells in a 15 ml polypropylene V-bottomed tube and added 30 μl of 3% FBS PBS, mixed cells well, and then added 1 ml of cold 80% ethenol. Fixed cells for 1 hour at 4° C. and washed cells twice in PBS. Added 0.5 ml of 50 μg/ml propidium iodide and PBS staining solution to cell pellet and mixed well. And added 300 μl of 2 mg/ml RNase A and incubated at dark room 1 hr at room temperature. Stored samples at 4° C. until analyzed by flow cytometry following standard methods and procedures.

Measurement of Cell Death

Cell culture was performed as described above. At 24, 48 and 72 hours culture media was collected to obtain dead cells and, and live cells harvested and single cell suspensions in PBS prepared using standard protocols. Cells suspensions were filtered to remove cell debris and PI was added to a final concentration of 50 μg/ml prior to FACS analysis. FACS gating was set to measure percentage of dead GFP positive cells (GFP⁺PI⁺) and dead GFP negative cells (GFP⁻PI⁺).

Measurement of Doxorubicin Induced Cell Death

Four cultures containing 1.5×10⁵ Hela cells and ZNF217-GFP transfected HeLa cells were set up in six well plates. Cells were cultured as described. At 48 hours 100 ng/ml, 200 ng/ml, and 500 ng/ml of doxorubicin was added to each. At 72 hours, culture supernatants were collected and single cell suspensions were prepared in 0.5 ml PBS, filtered, and 50 μg/ml PI added into the cell suspension for FACS analysis. FACS analysis was performed as described above.

Apoptosis Assay

1.5×10⁵ of Hela cells, ZNF217-GFP transfected HeLa cells, HBL100 cells and ZNF217-GFP transfected HBL100 cells were set up in six well plates and cultured as described above. One the second day, the cells were treated with 100 ng/ml doxirubicin for 16 hours. The cell culture supernatants and cells were collected and washed twice with PBS, resuspended in 100 μl of binding buffer (10 mM HEPS, 140 mM NaCl and 2.5 mM CaCl₂, pH 7.4). 5 μl of Annexin V antibody (Alexa Fluor 633 from Molecular Probes) was added and the cells were incubated at room temperature for 15 minutes. After the incubation, 400 μl of binding buffer were added and cell samples were analyzed by flow cytometry. A 633-nm wavelength laser was used to measure the apoptotic cell population. For imaging 1.5×10⁴ cells were seeded in 4 well cover glass slides (Fisher). Doxorubicin treating cells 16 hours later, the cells were washed with PBS twice and 200 μl of binding buffer were added, staining process was as same as above and in the meantime, 0.5 μM of DAPI were added to satin the cell nucleus. After staining, the cells were washed twice again with PBS, 400 μl of Binding buffer were added. The apoptotic cells were imaged in Seize con-focal image system.

Survival Assay

5.75×10⁵ of HBL100 and ZNF217-EGFP transfected HBL100 were plated in 100 mm plates and cultured as described above. On day two the cells was treated with 100 ng/ml of doxorubicin for 16 hours and washed twice with PBS and cultured continued in the fresh medium for eleven days. Cell numbers were counted with hemocytometer at day 3, day 5, day 8, day 11, separately.

II. Experimental Procedures

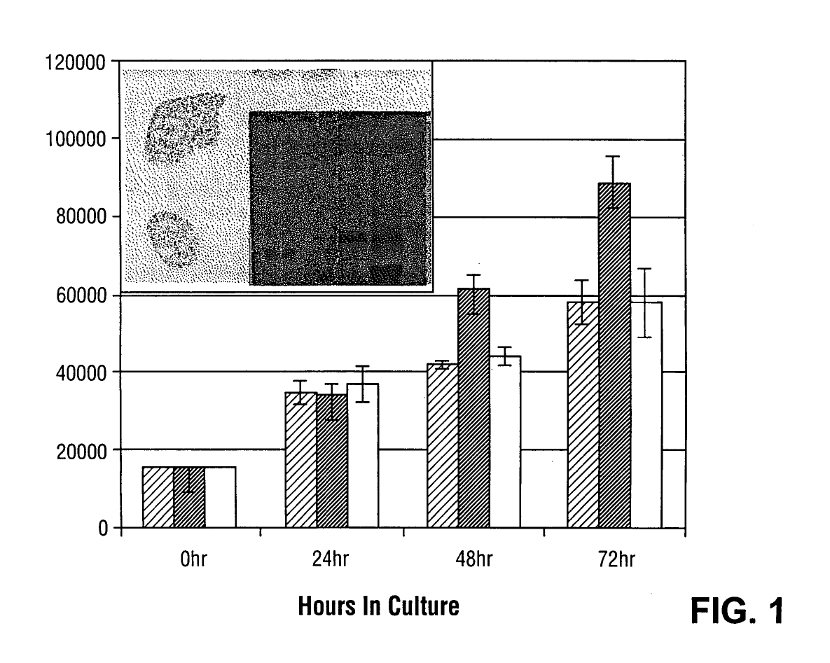

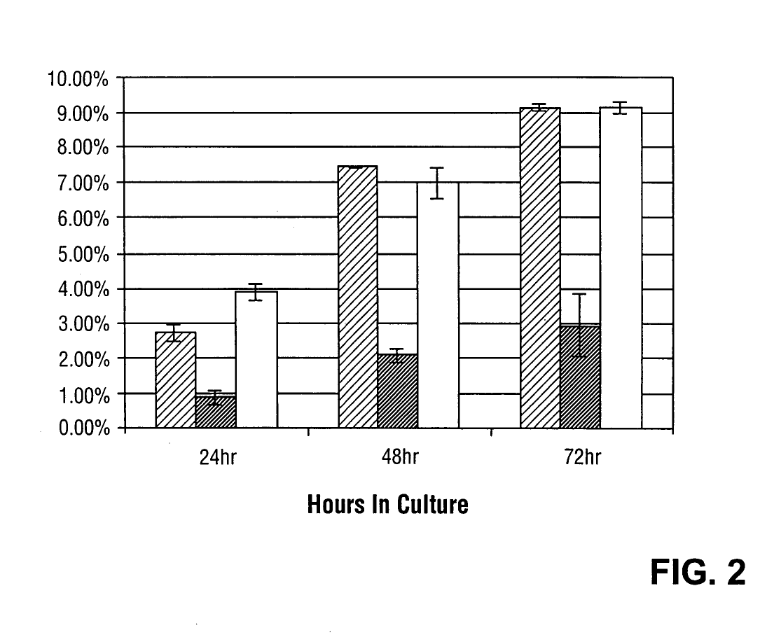

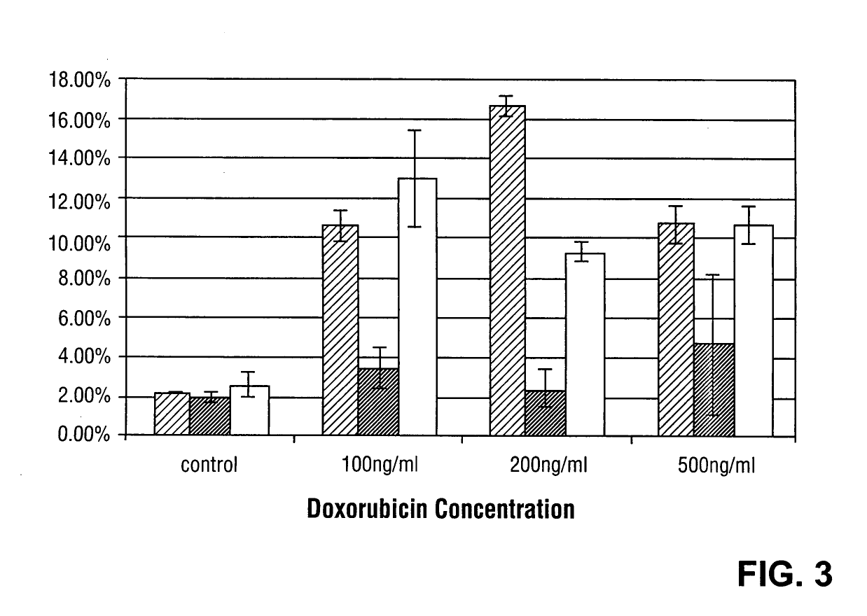

HeLa cells transfected with a plasmid encoding a ZNF217- Enhanced Green Fluorescent Protein (EGFP) fusion were observed to accumulate faster than parallel control cultures of non transfected HeLa cells or cells transfected with the EGFP vector alone. In independent experiments approximately 40% more cells were present in transfected cultures compared to the non-transfected cultures after 72 hours (FIG. 1). Transfected cells expressed the ZNF217-EGFP transcript at a level similar to the pathological levels of expression of ZNF217 observed in a subset of tumors (FIG. 1a, and Collins et al., 1998, supra). Next, a determination was made as to whether or not the increase in cell numbers was due to either an increase in cell proliferation or a decrease in apoptosis in ZNF217-EGFP transfected HeLa cells. Cell cycle analysis using fluorescent activated cell sorting (FACS) showed no difference in the cell cycle between the two populations of cells suggesting that the ZNF217-EGFP transfected HeLa cells do not grow faster than the control cells. The relative numbers of dead cells present at 24, 48 and 72 hours in parallel cultures of control and transfected HeLa cultures was quantified using FACS to count GFP+PI+cells. Independent experiments revealed that there was significantly less cell death in the transfected HeLa cells compared to the controls (FIG. 2) and that after 72 hours in culture there were ˜40% more transfected cells than control cells. Annexin V staining confirmed that cell death was due to apoptosis (FIG. 3). These data demonstrate that ZNF217 suppresses spontaneous apoptosis in cultured HeLa cells.

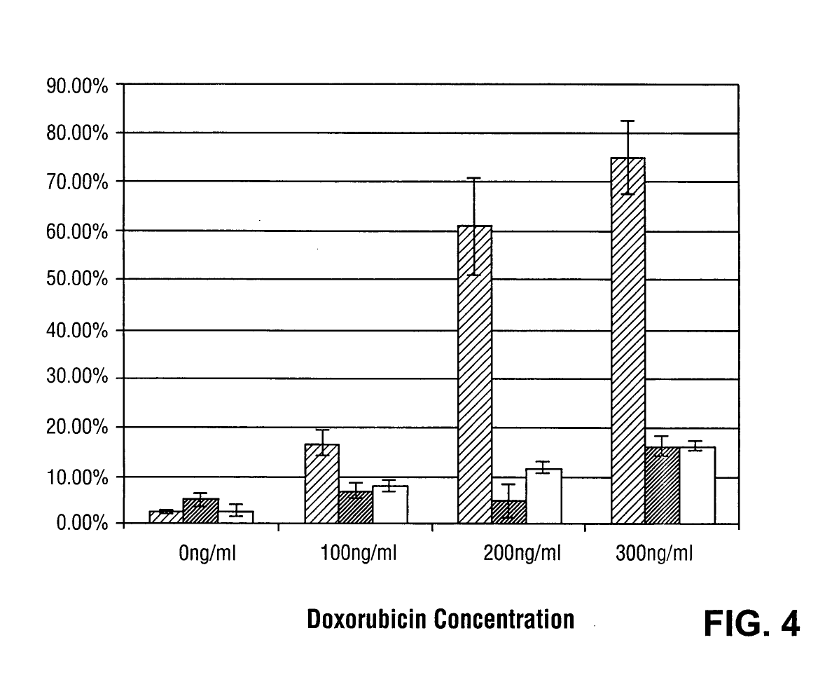

The ectopic expression of ZNF217 was then investigated. Could the ectopic expression of ZNF217 protect against doxorubicin-induced apoptosis in HeLa cells? Doxorubicin is a potent chemotherapeutic agent used in ˜40% of breast cancer patients. The efficacy of doxorubicin is due to its ability to inhibit topoisomerase II and induce double strand DNA breaks (DSB) resulting in ATM/p53 mediated apoptosis (Chabner, 1996, supra). In multiple independent experiments transfection of HeLa cells with ZNF217 was found to confer a 3 to 5-fold resistance of these cells to doxorubicin (FIG. 4). This suggests that the increased expression of ZNF217 in tumors is conferring on them an increased resistance to doxorubicin.

If ZNF217 confers resistance to doxorubicin then breast cancer cell lines in which ZNF217 is highly expressed should be more resistant to doxorubicin than breast cancer cell lines that express little ZNF217. A set of breast cancer cell lines that have known levels of ZNF217 expression were exposed to doxorubicin. MCF7 has high-level amplification of the ZNF217 region, resulting in over expression of the ZNF217 gene, whereas 600MPE has high-level expression but normal copy number of the locus, and HBL100 has relatively low levels of expression of ZNF217 (Collins et al., 1998, supra). All three cell lines have wild type p53. HBL100 showed higher levels of cell death than MCF7 and 600MPE upon exposure to doxorubicin (FIG. 4). To confirm that the resistance to doxorubicin was due to ZNF217 expression ZNF217 was transfected into wild type HBL100. Fluorescent activated cell sorted ZNF217-EGFP positive HBL100 cells and control HBL100 cultures were exposed to doxorubicin for 72 hours. Transfection of HBL100 cells with ZNF217-EGFP conferred a 3 to 5-fold resistance to doxorubicin in concentrations of 100, 200 and 300 ng/ml (FIG. 4). This shows that high level ZNF217 expression alone can protect cells from doxorubicin-induced apoptosis.

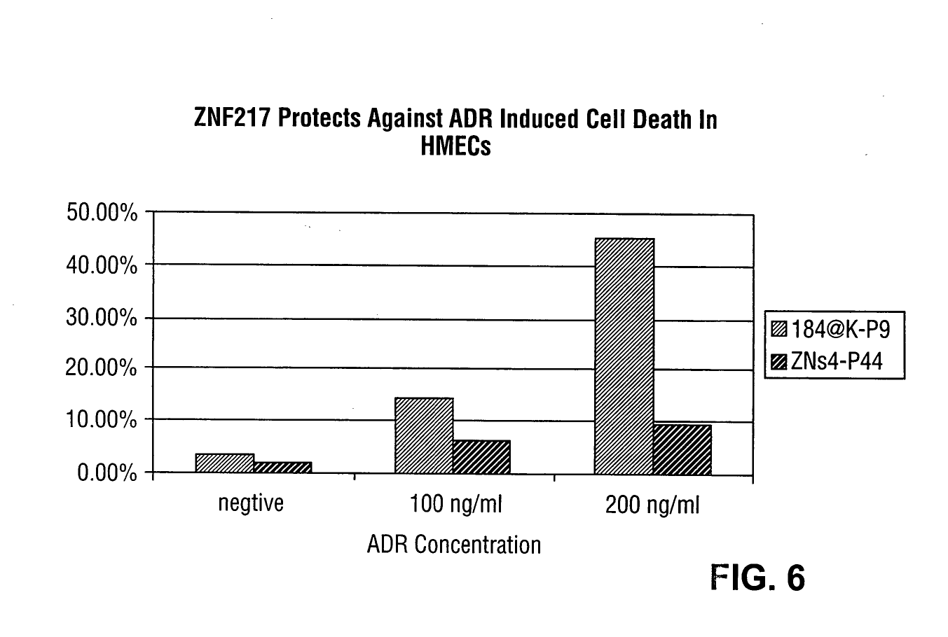

In order to determine if ZNF217 could confer resistance to doxorubicin on primary mammary epithelial cells, the sensitivity of HMECs which had been previously immortalized by a ZNF217-HA expression construct (Nonet, G. H. et al., 2001, supra) were compared to parental 184 HMECs with finite life span. Transduced HMEC expressed significantly more ZNF217 protein than non-transduced 184 HMEC. When the cultures were exposed to 100 ng/ml doxorubicin cells transduced with ZNF217 exhibited significantly (p=0.0146) less cell death (2.5-fold) than parallel cultures of control non-transduced 184 HMEC (FIG. 6). Thus, as well as promoting HMEC immortalization, ZNF217 expression protects HMEC against doxorubicin-induced apoptosis.

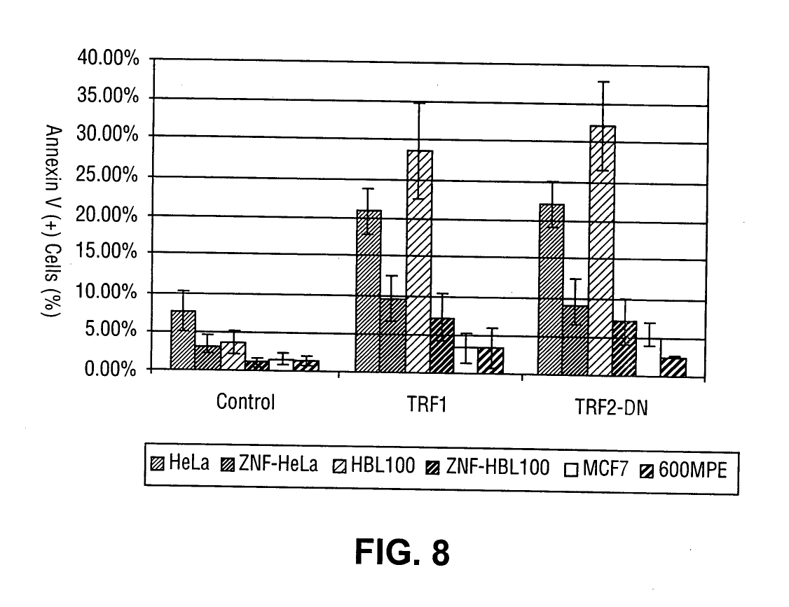

Senescent HMEC with short telomeres continue to divide with concomitant cell death (Romanov, S. R. et al., 2001, Nature 409:633-7). It is probable that cell death is the consequence of telomere-based crisis. If ZNF217 can attenuate an apoptotic signal emanating from critically short telomeres then over expressing clones may escape senescence to become immortal. To address this, the ability of ZNF217 to protect against telomere dysfunction induced apoptosis was investigated. HeLa and HBL100 cells were transfected with a dominant negative TRF2 mutant that functions to deprotect telomere ends and triggers ATM/p53 dependent apoptosis in vitro (Karlseder, J. et al., 1999, supra). In HeLa and HBL100 cells transfected with the TRF2 mutant apoptosis was induced, however, in ZNF217-EGFP HeLa and HBL100 double transfectants, the level of apoptosis was reduced 4 to 5-fold (FIG. 8). Furthermore, it was observed that TRF2 induced significantly less apoptosis when transfected into the breast cancer cell lines MCF7 and 600MPE which express high endogenous levels of ZNF217 than it induced in either wild type HBL100 or HeLa cells (FIG. 8A). In addition to TRF2-based telomere crisis, the ATM/p53 DNA damage response pathway is also induced by the double strand breaks which arise upon exposure to agents such as doxorubicin (Karlseder, J. et al., 1999, supra; Chin, L. et al., 1999, Cell 97: 527-538; Lee, K.-H. et al., 2001, Proc Natl Acad Sci U.S.A. 98:3381-6; and others). These results suggest that ZNF217 can both immortalize HMEC and confer resistance to doxorubicin by suppressing the ATM/p53 damage response pathway). TRF1 is a negative regulator of telomere length (van Steensel, B. et al., 1998, Cell 92:401-13) and its over expression elicits ATM/p53 independent apoptosis in cells with short telomeres (Kishi, S. et al., 2001, Oncogene 20:1497-508). ZNF217-EGFP was transfected into cell lines, and showed that ZNF217 also conferred significant protection against TRF1 induced apoptosis (FIG. 8B). Thus, ZNF217 can protect against independent cell death pathways triggered by telomere dysfunction.

It is known that disease free survival of breast cancer decreases by 50% in those women with amplification of the ZNF217 gene. In order to gain insight as to how ZNF217 expression levels might directly affect the ability of tumors to survive prolonged exposure to chemotherapeutic agents a chemotheraputic regimen was emulated in vitro. Parallel cultures containing equal numbers of HBL100 and HBL100 transfected with ZNF217-EGFP were exposed to doxorubicin for sixteen hours and then cultured in the absence of doxorubicin for eleven days. Cell numbers were determined on days 3, 5, 8, and 11. Both cultures showed approximately equal decline in cell number for eight days. This apparent loss of protection against apoptosis in the ZNF217-EGFP culture was due to all cells being counted, including non-transfected cells. However on day 11 ZNF217-EGFP transfected HBL100 cells showed a recovery with approximately 3.35-fold more cells than non-transfected HBL100.

IV. Discussion

The ZNF217 gene locus at 20 q13.2 is amplified in approximately 20% to 30% of early stage breast tumors (Waldman et al., 2000, supra). High level amplification of the locus is associated with a 50% decrease in disease free survival (Courjal, F. et al., 1996, supra). Increased 20q13.2 copy number is observed upon human papillomavirus immortalization of uroepithelial cells (Cuthill, S. et al., 1999, supra; Tanner, M. M. et al., 1995, supra) and kerotinocytes (Solinas-Toldo, S. et al., 1997, supra). Ectopic expression of ZNF217 results in the immortalization HMECs, which show low levels of endogenous ZNF217 expression. Importantly, immortalization occurs without an increase in 20q13.2 copy number (Nonet, G. H. et al., 2001, supra). The ZNF217 gene product resembles a kruppel-like transcription factor (Collins, C. et al., 1998, supra), localizes predominantly to the nucleus (Collins, C. et al., 2001, supra) and coimmunoprecipitates with histone deacetylase 1 (HDAC1) (You, A. et al., 2001, Proc Natl Acad Sci U S A 98:1454-8) suggesting that it may function as a transcriptional repressor.

Mammary epithelial cells senesce in two stages. The first stage termed “crisis” is characterized by a lack of cell division, intact checkpoints, a stable genome, and no detectable telomerase activity. The second stage termed “agonescence” is a terminal block to immortalization and is characterized by telomere erosion leading to telomere-based crisis, genome instability and cell division balanced by cell death (Romanov et al., 2001, supra). Ectopic expression of ZNF217 allows a subpopulation of HMEC to reactivate telomerase and to escape agonescence (Nonet et al., 2001, supra). In agonescence very short telomeres are thought to provoke apoptosis by exposing the telomeric DNA normally shielded by TRF2 (Griffith, J. D. et al., 1999, Cell 97:503-14) or by increasing the concentration of TRF1 not sequestered by the telomeres (Kishi, S. et al., 2001, supra). ZNF217 could escape from senescence and growth beyond agonescence by suppressing the apoptotic signals that emanate from critically short telomeres (Hahn, W. C. et al., 1999, Nat Med 5:1164-70; Kondo, S. et al., 1998, Oncogene 16:3323-30; Zhang, X. et al., 1999, Genes Dev 13:2388-99). The data supporting this hypothesis comes from two observations.

First, ZNF217 suppresses apoptosis when TRF2 is functionally inactivated in HBL100 and HeLa cells. Functional inactivation of TRF2 is known to lead to telomere-based crisis and ATM/p53 dependent apoptosis in HeLa, MCF7 and other cell lines (Karlseder et al., 1999, supra). Although p53 function in HeLa cells is compromised by HPV16 E6 it is nonetheless stabilized in response to TRF2 and functions properly as a transcription factor (Karlseder et al., 1999, supra). Current models suggest that functional inactivation of TRF2 exposes the termini of telomeres activating the ATM/p53 DNA damage checkpoint. The data show over expression of ZNF217 in TRF2- cells reduces cell death. Second, ZNF217 suppresses apoptosis triggered by overexpression of TRF 1. TRF 1 is a negative regulator of telomere length (van Steensel et al., 1998, supra) and its overexpression in cell lines with short telomeres, including HeLa, results in rapid apoptosis (Kishi et al, 2001, supra). It is believed the ratio of bound to unbound TRF I is critical since high levels of free TRF1 induce apoptosis. TRF1 signals apoptosis through an ATM/p53 independent pathway. Thus, ZNF217 can attenuate independent and redundant apoptotic signaling pathways resulting from telomere dysfunction.

Agonescent HMEC divide in the absence of telomerase with critically short telomeres and proliferation is accompanied by cell death, which blocks immortalization (Romanov et al., 2001). Thus, suppression of telomere dysfunction induced apoptosis might allow survival of proliferating HMEC with destabilized genomes. Evidence that ZNF217 promotes survival of HMEC with rearranged genomes comes from the fact that ZNF217 immortalized HMEC contain numerous genome aberrations not present in the parental HMEC (Nonet et al, 2001, supra).

It is important to determine what aspect of ZNF217 function accounts for the 50% decrease in disease-free survival for women whose tumors have high-level amplification at the ZNF217 locus (Courjal et al., 1996, supra; Tanner et al., 1995, supra). It is therefore significant that ZNF217 can effectively suppress doxorubicin-induced apoptosis in HeLa and HBL100 cells and, moreover, that in cell lines, sensitivity to doxorubicin is correlated with endogenous ZNF217 expression level. Doxorubicin is administered to approximately 40% of breast cancer patients. The efficacy of doxorubicin derives from its ability to inhibit topoisomerase II resulting in DSBs and ATM/p53 mediated apoptosis (Chabner, 1996, supra). The ability of ZNF217 to suppress doxorubicin-induced apoptosis can explain the 50% decrease in disease-free survival for women having tumors with amplification of the ZNF217 gene.

Because aberrant expression of ZNF217 can occur even with normal copy number (Collins et al., 1998; 2001, both supra), functional polymorphisms that increase either ZNF217 expression or activity could modulate susceptibility to breast cancer and/or effectiveness of chemotherapy. Finally, inactivation of ZNF217 function in tumors with normal ZNF217 protein abundance could render these tumors exquisitely sensitive to existing anti tumor therapies. Finally, inactivation of ZNF217 function in tumors with normal ZNF217 protein abundance may render these tumors exquisitely sensitive to existing anti tumor therapies.

### Example 2

ZNF217 silencing RNA (siRNA) constructs were introduced into HBL100 cells transfected with znf217 and the cells were subsequently treated with Doxorubicin.

The siRNA Dicer Generation kit from Gene Therapy System was used to generate a series of siRNAs. Approximately 30 different 22 bp siRNA sequences were generated, using the following primers:

T7 dicer sense (SEQ ID NO: 4)
5′ (T7 Promoter)-CGTTGCTGGGAAAAGATGTG 3′
(2547-2567 of ZNF217)
B: T7 dicer antisense (SEQ ID NO: 5)
5′ (T7 Promoter)-GCGGTAACAGTGATGTGATG 3′
(3119-3140 of Znf217)

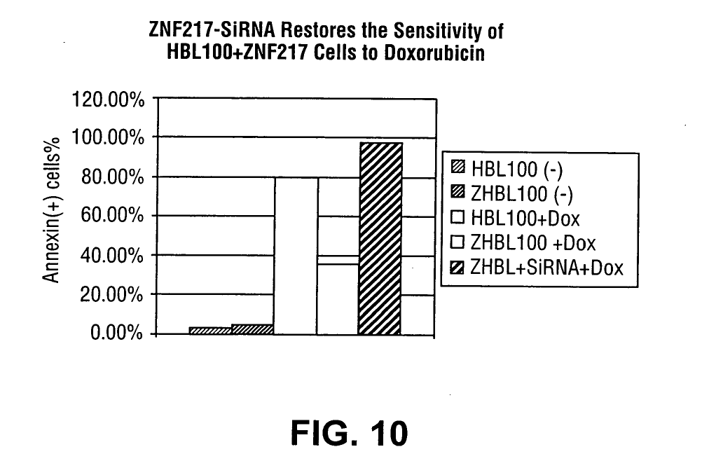

As demonstrated in FIG. 10, cells transformed with the siRNA constructs displayed increased annexin staining in the presences of Doxorubicin compared to control cells. There results further demonstrate that inhibition of ZN217 potentiates chemotherapy.

### Example 3

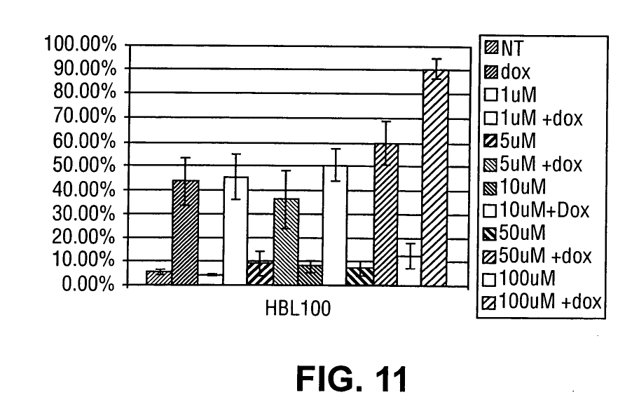

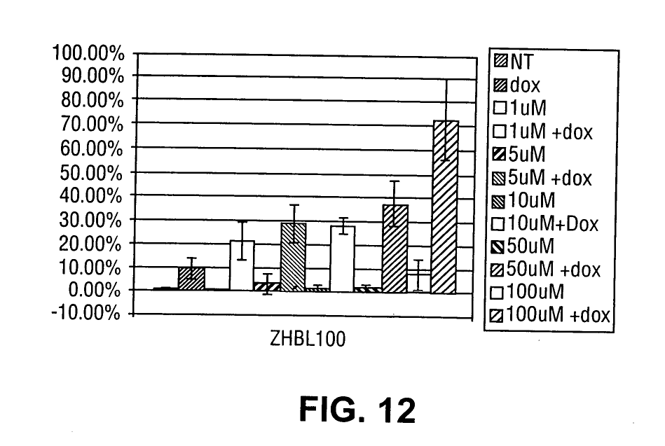

Small molecule inhibitors of ZNF217 also act in synergy with chemotherapeutic drugs to treat cancer cells. HBL100 cells (either transfected with ZNF217-encoding constructs (FIG. 12) or not (FIG. 11)) were treated with triciribine phosphate. As displayed in FIGS. 11 and 12, increasing concentrations of triciribine phosphate, in combination with Doxorubicon, resulted in increased cell death. There results further demonstrate that inhibition of ZN217 potentiates chemotherapy.

It is understood that the examples and embodiments described herein are for illustrative purposes only and that various modifications or changes in light thereof will be suggested to persons skilled in the art and are to be included within the spirit and purview of this application and scope of the appended claims.

All publications, patents and patent applications cited herein are hereby incorporated by reference in their entireties for all purposes to the same extent as if each individual publication, patent or patent application were specifically and individually indicated to be so incorporated by reference.

What is claimed is:

1. A method of determining a course of treatment for a cancer patient, the method comprising,
detecting the level of a ZNF217 polypeptide or polynucleotide in a biological sample from the patient; and
selecting an anti-cancer treatment for the patient, wherein a non-increased level of the ZNF217 polypeptide or polynucleotide in the sample indicates that the patient can be effectively treated with a topoisomerase II inhibitor, thereby determining a course of treatment for the cancer patient.

2. The method of claim 1, wherein the anti-cancer treatment comprises administration of a chemotherapeutic agent.

3. The method of claim 1, wherein the level of the ZNF217 polypeptide or polynucleotide is not increased compared to a control sample and doxorubicin is administered to the patient.

4. The method of claim 1, wherein the level of the ZNF217 polynucleotide is detected.

5. The method of claim 1, wherein the level of the ZNF217 polypeptide is detected.

6. A method of monitoring the efficacy of a cancer treatment, the method comprising detecting the level of a ZNF217 polypeptide or polynucleotide in a biological sample from a patent undergoing treatment for cancer, wherein a reduced level of the ZNF217 polypeptide or polynucleotide in the biological sample compared to the level in a biological sample from the patient prior to, or earlier in, the treatment is indicative of efficacious treatment.

7. The method of claim 6, wherein the cancer is breast cancer.

8. The method of claim 6, wherein the treatment comprises administration of a chemotherapeutic agent.

9. The method of claim 6, wherein the level of the ZNF217 polynucleotide is detected.

10. The method of claim 6, wherein the level of the ZNF217 polypeptide is detected.

11. The method of claim 6, comprising detecting the ZNF217 polynucleotide or polypeptide levels in samples from the patient who subsequently undergoes an anti-cancer therapy, and correlating the ZNF217 polynucleotide or polypeptide levels with the known efficacy of the anti-cancer therapy.


SEQUENCE LISTING (5 sequences)

SEQ ID NO: 1 - protein, 6 aa, Artificial Sequence
  Description of Artificial Sequence6-His epitope tag
HHHHHH

SEQ ID NO: 2 - protein, 8 aa, Artificial Sequence
  Description of Artificial Sequenceanti- DYKDDDDK epitope tag
DYKDDDDK

SEQ ID NO: 3 - protein, 200 aa, Artificial Sequence
  Description of Artificial Sequencepoly-Gly flexible linker
  MOD_RES (6)..(200): Gly residues from position 6 to 200 may be present or absent
GGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGG

SEQ ID NO: 4 - DNA, 20 bp, Artificial Sequence
  Description of Artificial SequenceT7 dicer sense primer (2547-2567 of ZNF217)
cgttgctgggaaaagatgtg

SEQ ID NO: 5 - DNA, 20 bp, Artificial Sequence
  Description of Artificial SequenceT7 dicer antisense primer (3119-3140 of ZNF217)
gcggtaacagtgatgtgatg